SEQUENCE LISTING (68 sequences)

SEQ ID NO: 1 - protein, 360 aa, Homo sapiens
MAGHLASDFAFSPPPGGGGDGPGGPEPGWVDPRTWLSFQGPPGGPGIGPGVGPGSEVWGIPPCPPPYEFCGGMAYCGPQVGVGLVPQGGLETSQPEGEAGVGVESNSDGASPEPCTVTPGAVKLEKEKLEQNPEESQDIKALQKELEQFAKLLKQKRITLGYTQADVGLTLGVLFGKVFSQTTICRFEALQLSFKNMCKLRPLLQKWVEEADNNENLQEICKAETLVQARKRKRTSIENRVRGNLENLFLQCPKPTLQQISHIAQQLGLEKDVVRVWFCNRRQKGKRSSSDYAQREDFEAAGSPFSGGPVSFPLAPGPHFGTPGYGSPHFTALYSSVPFPEGEAFPPVSVTTLGSPMHSN

SEQ ID NO: 2 - protein, 317 aa, Homo sapiens
MYNMMETELKPPGPQQTSGGGGGNSTAAAAGGNQKNSPDRVKRPMNAFMVWSRGQRRKMAQENPKMHNSEISKRLGAEWKLLSETEKRPFIDEAKRLRALHMKEHPDYKYRPRRKTKTLMKKDKYTLPGGLLAPGGNSMASGVGVGAGLGAGVNQRMDSYAHMNGWSNGSYSMMQDQLGYPQHPGLNAHGAAQMQPMHRYDVSALQYNSMTSSQTYMNGSPTYSMSYSQQGTPGMALGSMGSVVKSEASSSPPVVTSSSHSRAPCQAGDLRDMISMYLPGAEVPEPAAPSRLHMSQHYQSGPVPGTAINGTLPLSHM

SEQ ID NO: 3 - protein, 470 aa, Homo sapiens
MAVSDALLPSFSTFASGPAGREKTLRQAGAPNNRWREELSHMKRLPPVLPGRPYDLAAATVATDLESGGAGAACGGSNLAPLPRRETEEFNDLLDLDFILSNSLTHPPESVAATVSSSASASSSSSPSSSGPASAPSTCSFTYPIRAGNDPGVAPGGTGGGLLYGRESAPPPTAPFNLADINDVSPSGGFVAELLRPELDPVYIPPQQPQPPGGGLMGKFVLKASLSAPGSEYGSPSVISVSKGSPDGSHPVVVAPYNGGPPRTCPKIKQEAVSSCTHLGAGPPLSNGHRPAAHDFPLGRQLPSRTTPTLGLEEVLSSRDCHPALPLPPGFHPHPGPNYPSFLPDQMQPQVPPLHYQELMPPGSCMPEEPKPKRGRRSWPRKRTATHTCDYAGCGKTYTKSSHLKAHLRTHTGEKPYHCDWDGCGWKFARSDELTRHYRKHTGHRPFQCQKCDRAFSRSDHLALHMKRHF

SEQ ID NO: 4 - protein, 454 aa, Homo sapiens
MDFFRVVENQQPPATMPLNVSFTNRNYDLDYDSVQPYFYCDEEENFYQQQQQSELQPPAPSEDIWKKFELLPTPPLSPSRRSGLCSPSYVAVTPFSLRGDNDGGGGSFSTADQLEMVTELLGGDMVNQSFICDPDDETFIKNIIIQDCMWSGFSAAAKLVSEKLASYQAARKDSGSPNPARGHSVCSTSSLYLQDLSAAASECIDPSVVFPYPLNDSSSPKSCASQDSSAFSPSSDSLLSSTESSPQGSPEPLVLHEETPPTTSSDSEEEQEDEEEIDVVSVEKRQAPGKRSESGSPSAGGHSKPPHSPLVLKRCHVSTHQHNYAAPPSTRKDYPAAKRVKLDSVRVLRQISNNRKCTSPRSSDTEENVKRRTHNVLERQRRNELKRSFFALRDQIPELENNEKAPKVVILKKATAYILSVQAEEQKLISEEDLLRKRREQLKHKLEQLRNSCA

SEQ ID NO: 5 - protein, 209 aa, Homo sapiens
MGSVSNQQFAGGCAKAAEEAPEEAPEDAARAADEPQLLHGAGICKWFNVRMGFGFLSMTARAGVALDPPVDVFVHQSKLHMEGFRSLKEGEAVEFTFKKSAKGLESIRVTGPGGVFCIGSERRPKGKSMQKRRSKGDRCYNCGGLDHHAKECKLPPQPKKCHFCQSISHMVASCPLKAQQGPSAQGKPTYFREEEEEIHSPTLLPEAQN

SEQ ID NO: 6 - protein, 305 aa, Homo sapiens
MSVDPACPQSLPCFEASDCKESSPMPVICGPEENYPSLQMSSAEMPHTETVSPLPSSMDLLIQDSPDSSTSPKGKQPTSAENSVAKKEDKVPVKKQKTRTVFSSTQLCVLNDRFQRQKYLSLQQMQELSNILNLSYKQVKTWFQNQRMKSKRWQKNNWPKNSNGVTQKASAPTYPSLYSSYHQGCLVNPTGNLPMWSNQTWNNSTWSNQTQNIQSWSNHSWNTQTWCTQSWNNQAWNSPFYNCGEESLQSCMQFQPNSPASDLEAALEAAGEGLNVIQQTTRYFSTPQTMDLFLNYSMNMQPEDV

SEQ ID NO: 7 - protein, 356 aa, Homo sapiens
MTKSYSESGLMGEPQPQGPPSWTDECLSSQDEEHEADKKEDDLEAMNAEEDSLRNGGEEEDEDEDLEEEEEEEEEDDDQKPKRRGPKKKKMTKARLERFKLRRMKANARERNRMHGLNAALDNLRKVVPCYSKTQKLSKIETLRLAKNYIWALSEILRSGKSPDLVSFVQTLCKGLSQPTTNLVAGCLQLNPRTFLPEQNQDMPPHLPTASASFPVHPYSYQSPGLPSPPYGTMDSSHVFHVKPPPHAYSAALEPFFESPLTDCTSPSFDGPLSPPLSINGNFSFKHEPSAEFEKNYAFTMHYPAATLAGAQSHGSIFSGTAAPRCEIPIDNIMSFDSHSHHERVMSAQLNAIFHD

SEQ ID NO: 8 - protein, 214 aa, Homo sapiens
MTPQPSGAPTVQVTRETERSFPRASEDEVTCPTSAPPSPTRTPGNCAEAEEGGCRGAPRKLRARRGGRSRPKSELALSKQRRSRRKKANDRERNRMHDLNSALDALRGVLPTFPDDAKLTKIETLRFAHNYIWALTQTLRIADHSLYALEPPAPHCGELGSPGGPPGDWGSLYSPVSQAGSLSPAASLEERPGLLGATSSACLSPGSLAFSDFL

SEQ ID NO: 9 - protein, 283 aa, Homo sapiens
MNGEEQYYAATQLYKDPCAFQRGPAPEFSASPPACLYMGRQPPPPPPHPFPGALGALEQGSPPDISPYEVPPLADDPAVAHLHHHLPAQLALPHPPAGPFPEGAEPGVLEEPNRVQLPFPWMKSTKAHAWKGQWAGGAYAAEPEENKRTRTAYTRAQLLELEKEFLFNKYISRPRRVELAVMLNLTERHIKIWFQNRRMKWKKEEDKKRGGGTAVGGGGVAEPEQDCAVTSGEELLALPPPPPPGGAVPPAAPVAAREGRLPPGLSASPQPSSVAPRRPQEPR

SEQ ID NO: 10 - protein, 352 aa, Homo sapiens
MAAELAMGAELPSSPLAIEYVNDFDLMKFEVKKEPPEAERFCHRLPPGSLSSTPLSTPCSSVPSSPSFCAPSPGTGGGGGAGGGGGSSQAGGAPGPPSGGPGAVGGTSGKPALEDLYWMSGYQHHLNPEALNLTPEDAVEALIGSGHHGAHHGAHHPAAAAAYEAFRGPGFAGGGGADDMGAGHHHGAHHAAHHHHAAHHHHHHHHHGGAGHGGGAGHHVRLEERFSDDQLVSMSVRELNRQLRGFSKEEVIRLKQKRRTLKNRGYAQSCRFKRVQQRHILESEKCQLQSQVEQLKLEVGRLAKERDLYKEKYEKLAGRGGPGSAGGAGFPREPSPPQAGPGGAKGTADFFL

SEQ ID NO: 11 - protein, 431 aa, Homo sapiens
MPNPRPGKPSAPSLALGPSPGASPSWRAAPKASDLLGARGPGGTFQGRDLRGGAHASSSSLNPMPPSQLQLPTLPLVMVAPSGARLGPLPHLQALLQDRPHFMHQLSTVDAHARTPVLQVHPLESPAMISLTPPTTATGVFSLKARPGLPPGINVASLEWVSREPALLCTFPNPSAPRKDSTLSAVPQSSYPLLANGVCKWPGCEKVFEEPEDFLKHCQADHLLDEKGRAQCLLQREMVQSLEQQLVLEKEKLSAMQAHLAGKMALTKASSVASSDKGSCCIVAAGSQGPVVPAWSGPREAPDSLFAVRRHLWGSHGNSTFPEFLHNMDYFKFHNMRPPFTYATLIRWAILEAPEKQRTLNEIYHWFTRMFAFFRNHPATWKNAIRHNLSLHKCFVRVESEKGAVWTVDELEFRKKRSQRPSRCSNPTPGP

SEQ ID NO: 12 - protein, 380 aa, Artificial Sequence
  transducible material
MAGHLASDFAFSPPPGGGGDGPGGPEPGWVDPRTWLSFQGPPGGPGIGPGVGPGSEVWGIPPCPPPYEFCGGMAYCGPQVGVGLVPQGGLETSQPEGEAGVGVESNSDGASPEPCTVTPGAVKLEKEKLEQNPEESQDIKALQKELEQFAKLLKQKRITLGYTQADVGLTLGVLFGKVFSQTTICRFEALQLSFKNMCKLRPLLQKWVEEADNNENLQEICKAETLVQARKRKRTSIENRVRGNLENLFLQCPKPTLQQISHIAQQLGLEKDVVRVWFCNRRQKGKRSSSDYAQREDFEAAGSPFSGGPVSFPLAPGPHFGTPGYGSPHFTALYSSVPFPEGEAFPPVSVTTLGSPMHSNESGGGGSPGRRRRRRRRRRR

SEQ ID NO: 13 - protein, 337 aa, Artificial Sequence
  transducible material
MYNMMETELKPPGPQQTSGGGGGNSTAAAAGGNQKNSPDRVKRPMNAFMVWSRGQRRKMAQENPKMHNSEISKRLGAEWKLLSETEKRPFIDEAKRLRALHMKEHPDYKYRPRRKTKTLMKKDKYTLPGGLLAPGGNSMASGVGVGAGLGAGVNQRMDSYAHMNGWSNGSYSMMQDQLGYPQHPGLNAHGAAQMQPMHRYDVSALQYNSMTSSQTYMNGSPTYSMSYSQQGTPGMALGSMGSVVKSEASSSPPVVTSSSHSRAPCQAGDLRDMISMYLPGAEVPEPAAPSRLHMSQHYQSGPVPGTAINGTLPLSHMESGGGGSPGRRRRRRRRRRR

SEQ ID NO: 14 - protein, 490 aa, Artificial Sequence
  transducible material
MAVSDALLPSFSTFASGPAGREKTLRQAGAPNNRWREELSHMKRLPPVLPGRPYDLAAATVATDLESGGAGAACGGSNLAPLPRRETEEFNDLLDLDFILSNSLTHPPESVAATVSSSASASSSSSPSSSGPASAPSTCSFTYPIRAGNDPGVAPGGTGGGLLYGRESAPPPTAPFNLADINDVSPSGGFVAELLRPELDPVYIPPQQPQPPGGGLMGKFVLKASLSAPGSEYGSPSVISVSKGSPDGSHPVVVAPYNGGPPRTCPKIKQEAVSSCTHLGAGPPLSNGHRPAAHDFPLGRQLPSRTTPTLGLEEVLSSRDCHPALPLPPGFHPHPGPNYPSFLPDQMQPQVPPLHYQELMPPGSCMPEEPKPKRGRRSWPRKRTATHTCDYAGCGKTYTKSSHLKAHLRTHTGEKPYHCDWDGCGWKFARSDELTRHYRKHTGHRPFQCQKCDRAFSRSDHLALHMKRHFESGGGGSPGRRRRRRRRRRR

SEQ ID NO: 15 - protein, 474 aa, Artificial Sequence
  transducible material
MDFFRVVENQQPPATMPLNVSFTNRNYDLDYDSVQPYFYCDEEENFYQQQQQSELQPPAPSEDIWKKFELLPTPPLSPSRRSGLCSPSYVAVTPFSLRGDNDGGGGSFSTADQLEMVTELLGGDMVNQSFICDPDDETFIKNIIIQDCMWSGFSAAAKLVSEKLASYQAARKDSGSPNPARGHSVCSTSSLYLQDLSAAASECIDPSVVFPYPLNDSSSPKSCASQDSSAFSPSSDSLLSSTESSPQGSPEPLVLHEETPPTTSSDSEEEQEDEEEIDVVSVEKRQAPGKRSESGSPSAGGHSKPPHSPLVLKRCHVSTHQHNYAAPPSTRKDYPAAKRVKLDSVRVLRQISNNRKCTSPRSSDTEENVKRRTHNVLERQRRNELKRSFFALRDQIPELENNEKAPKVVILKKATAYILSVQAEEQKLISEEDLLRKRREQLKHKLEQLRNSCAESGGGGSPGRRRRRRRRRRR

SEQ ID NO: 16 - protein, 229 aa, Artificial Sequence
  transducible material
MGSVSNQQFAGGCAKAAEEAPEEAPEDAARAADEPQLLHGAGICKWFNVRMGFGFLSMTARAGVALDPPVDVFVHQSKLHMEGFRSLKEGEAVEFTFKKSAKGLESIRVTGPGGVFCIGSERRPKGKSMQKRRSKGDRCYNCGGLDHHAKECKLPPQPKKCHFCQSISHMVASCPLKAQQGPSAQGKPTYFREEEEEIHSPTLLPEAQNESGGGGSPGRRRRRRRRRRR

SEQ ID NO: 17 - protein, 325 aa, Artificial Sequence
  transducible material
MSVDPACPQSLPCFEASDCKESSPMPVICGPEENYPSLQMSSAEMPHTETVSPLPSSMDLLIQDSPDSSTSPKGKQPTSAENSVAKKEDKVPVKKQKTRTVFSSTQLCVLNDRFQRQKYLSLQQMQELSNILNLSYKQVKTWFQNQRMKSKRWQKNNWPKNSNGVTQKASAPTYPSLYSSYHQGCLVNPTGNLPMWSNQTWNNSTWSNQTQNIQSWSNHSWNTQTWCTQSWNNQAWNSPFYNCGEESLQSCMQFQPNSPASDLEAALEAAGEGLNVIQQTTRYFSTPQTMDLFLNYSMNMQPEDVESGGGGSPGRRRRRRRRRRR

SEQ ID NO: 18 - protein, 376 aa, Artificial Sequence
  transducible material
MTKSYSESGLMGEPQPQGPPSWTDECLSSQDEEHEADKKEDDLEAMNAEEDSLRNGGEEEDEDEDLEEEEEEEEEDDDQKPKRRGPKKKKMTKARLERFKLRRMKANARERNRMHGLNAALDNLRKVVPCYSKTQKLSKIETLRLAKNYIWALSEILRSGKSPDLVSFVQTLCKGLSQPTTNLVAGCLQLNPRTFLPEQNQDMPPHLPTASASFPVHPYSYQSPGLPSPPYGTMDSSHVFHVKPPPHAYSAALEPFFESPLTDCTSPSFDGPLSPPLSINGNFSFKHEPSAEFEKNYAFTMHYPAATLAGAQSHGSIFSGTAAPRCEIPIDNIMSFDSHSHHERVMSAQLNAIFHDESGGGGSPGRRRRRRRRRRR

SEQ ID NO: 19 - protein, 234 aa, Artificial Sequence
  transducible material
MTPQPSGAPTVQVTRETERSFPRASEDEVTCPTSAPPSPTRTPGNCAEAEEGGCRGAPRKLRARRGGRSRPKSELALSKQRRSRRKKANDRERNRMHDLNSALDALRGVLPTFPDDAKLTKIETLRFAHNYIWALTQTLRIADHSLYALEPPAPHCGELGSPGGPPGDWGSLYSPVSQAGSLSPAASLEERPGLLGATSSACLSPGSLAFSDFLESGGGGSPGRRRRRRRRRRR

SEQ ID NO: 20 - protein, 303 aa, Artificial Sequence
  transducible material
MNGEEQYYAATQLYKDPCAFQRGPAPEFSASPPACLYMGRQPPPPPPHPFPGALGALEQGSPPDISPYEVPPLADDPAVAHLHHHLPAQLALPHPPAGPFPEGAEPGVLEEPNRVQLPFPWMKSTKAHAWKGQWAGGAYAAEPEENKRTRTAYTRAQLLELEKEFLFNKYISRPRRVELAVMLNLTERHIKIWFQNRRMKWKKEEDKKRGGGTAVGGGGVAEPEQDCAVTSGEELLALPPPPPPGGAVPPAAPVAAREGRLPPGLSASPQPSSVAPRRPQEPRESGGGGSPGRRRRRRRRRRR

SEQ ID NO: 21 - protein, 372 aa, Artificial Sequence
  transducible material
MAAELAMGAELPSSPLAIEYVNDFDLMKFEVKKEPPEAERFCHRLPPGSLSSTPLSTPCSSVPSSPSFCAPSPGTGGGGGAGGGGGSSQAGGAPGPPSGGPGAVGGTSGKPALEDLYWMSGYQHHLNPEALNLTPEDAVEALIGSGHHGAHHGAHHPAAAAAYEAFRGPGFAGGGGADDMGAGHHHGAHHAAHHHHAAHHHHHHHHHGGAGHGGGAGHHVRLEERFSDDQLVSMSVRELNRQLRGFSKEEVIRLKQKRRTLKNRGYAQSCRFKRVQQRHILESEKCQLQSQVEQLKLEVGRLAKERDLYKEKYEKLAGRGGPGSAGGAGFPREPSPPQAGPGGAKGTADFFLESGGGGSPGRRRRRRRRRRR

SEQ ID NO: 22 - protein, 451 aa, Artificial Sequence
  transducible material
MPNPRPGKPSAPSLALGPSPGASPSWRAAPKASDLLGARGPGGTFQGRDLRGGAHASSSSLNPMPPSQLQLPTLPLVMVAPSGARLGPLPHLQALLQDRPHFMHQLSTVDAHARTPVLQVHPLESPAMISLTPPTTATGVFSLKARPGLPPGINVASLEWVSREPALLCTFPNPSAPRKDSTLSAVPQSSYPLLANGVCKWPGCEKVFEEPEDFLKHCQADHLLDEKGRAQCLLQREMVQSLEQQLVLEKEKLSAMQAHLAGKMALTKASSVASSDKGSCCIVAAGSQGPVVPAWSGPREAPDSLFAVRRHLWGSHGNSTFPEFLHNMDYFKFHNMRPPFTYATLIRWAILEAPEKQRTLNEIYHWFTRMFAFFRNHPATWKNAIRHNLSLHKCFVRVESEKGAVWTVDELEFRKKRSQRPSRCSNPTPGPESGGGGSPGRRRRRRRRRRR

SEQ ID NO: 23 - protein, 400 aa, Artificial Sequence
  transducible material
MGSSHHHHHHSSGLVPRGSHMAGHLASDFAFSPPPGGGGDGPGGPEPGWVDPRTWLSFQGPPGGPGIGPGVGPGSEVWGIPPCPPPYEFCGGMAYCGPQVGVGLVPQGGLETSQPEGEAGVGVESNSDGASPEPCTVTPGAVKLEKEKLEQNPEESQDIKALQKELEQFAKLLKQKRITLGYTQADVGLTLGVLFGKVFSQTTICRFEALQLSFKNMCKLRPLLQKWVEEADNNENLQEICKAETLVQARKRKRTSIENRVRGNLENLFLQCPKPTLQQISHIAQQLGLEKDVVRVWFCNRRQKGKRSSSDYAQREDFEAAGSPFSGGPVSFPLAPGPHFGTPGYGSPHFTALYSSVPFPEGEAFPPVSVTTLGSPMHSNESGGGGSPGRRRRRRRRRRR

SEQ ID NO: 24 - protein, 357 aa, Artificial Sequence
  transducible material
MGSSHHHHHHSSGLVPRGSHMYNMMETELKPPGPQQTSGGGGGNSTAAAAGGNQKNSPDRVKRPMNAFMVWSRGQRRKMAQENPKMHNSEISKRLGAEWKLLSETEKRPFIDEAKRLRALHMKEHPDYKYRPRRKTKTLMKKDKYTLPGGLLAPGGNSMASGVGVGAGLGAGVNQRMDSYAHMNGWSNGSYSMMQDQLGYPQHPGLNAHGAAQMQPMHRYDVSALQYNSMTSSQTYMNGSPTYSMSYSQQGTPGMALGSMGSVVKSEASSSPPVVTSSSHSRAPCQAGDLRDMISMYLPGAEVPEPAAPSRLHMSQHYQSGPVPGTAINGTLPLSHMESGGGGSPGRRRRRRRRRRR

SEQ ID NO: 25 - protein, 510 aa, Artificial Sequence
  transducible material
MGSSHHHHHHSSGLVPRGSHMAVSDALLPSFSTFASGPAGREKTLRQAGAPNNRWREELSHMKRLPPVLPGRPYDLAAATVATDLESGGAGAACGGSNLAPLPRRETEEFNDLLDLDFILSNSLTHPPESVAATVSSSASASSSSSPSSSGPASAPSTCSFTYPIRAGNDPGVAPGGTGGGLLYGRESAPPPTAPFNLADINDVSPSGGFVAELLRPELDPVYIPPQQPQPPGGGLMGKFVLKASLSAPGSEYGSPSVISVSKGSPDGSHPVVVAPYNGGPPRTCPKIKQEAVSSCTHLGAGPPLSNGHRPAAHDFPLGRQLPSRTTPTLGLEEVLSSRDCHPALPLPPGFHPHPGPNYPSFLPDQMQPQVPPLHYQELMPPGSCMPEEPKPKRGRRSWPRKRTATHTCDYAGCGKTYTKSSHLKAHLRTHTGEKPYHCDWDGCGWKFARSDELTRHYRKHTGHRPFQCQKCDRAFSRSDHLALHMKRHFESGGGGSPGRRRRRRRRRRR

SEQ ID NO: 26 - protein, 494 aa, Artificial Sequence
  transducible material
MGSSHHHHHHSSGLVPRGSHMDFFRVVENQQPPATMPLNVSFTNRNYDLDYDSVQPYFYCDEEENFYQQQQQSELQPPAPSEDIWKKFELLPTPPLSPSRRSGLCSPSYVAVTPFSLRGDNDGGGGSFSTADQLEMVTELLGGDMVNQSFICDPDDETFIKNIIIQDCMWSGFSAAAKLVSEKLASYQAARKDSGSPNPARGHSVCSTSSLYLQDLSAAASECIDPSVVFPYPLNDSSSPKSCASQDSSAFSPSSDSLLSSTESSPQGSPEPLVLHEETPPTTSSDSEEEQEDEEEIDVVSVEKRQAPGKRSESGSPSAGGHSKPPHSPLVLKRCHVSTHQHNYAAPPSTRKDYPAAKRVKLDSVRVLRQISNNRKCTSPRSSDTEENVKRRTHNVLERQRRNELKRSFFALRDQIPELENNEKAPKVVILKKATAYILSVQAEEQKLISEEDLLRKRREQLKHKLEQLRNSCAESGGGGSPGRRRRRRRRRRR

SEQ ID NO: 27 - protein, 249 aa, Artificial Sequence
  transducible material
MGSSHHHHHHSSGLVPRGSHMGSVSNQQFAGGCAKAAEEAPEEAPEDAARAADEPQLLHGAGICKWFNVRMGFGFLSMTARAGVALDPPVDVFVHQSKLHMEGFRSLKEGEAVEFTFKKSAKGLESIRVTGPGGVFCIGSERRPKGKSMQKRRSKGDRCYNCGGLDHHAKECKLPPQPKKCHFCQSISHMVASCPLKAQQGPSAQGKPTYFREEEEEIHSPTLLPEAQNESGGGGSPGRRRRRRRRRRR

SEQ ID NO: 28 - protein, 345 aa, Artificial Sequence
  transducible material
MGSSHHHHHHSSGLVPRGSHMSVDPACPQSLPCFEASDCKESSPMPVICGPEENYPSLQMSSAEMPHTETVSPLPSSMDLLIQDSPDSSTSPKGKQPTSAENSVAKKEDKVPVKKQKTRTVFSSTQLCVLNDRFQRQKYLSLQQMQELSNILNLSYKQVKTWFQNQRMKSKRWQKNNWPKNSNGVTQKASAPTYPSLYSSYHQGCLVNPTGNLPMWSNQTWNNSTWSNQTQNIQSWSNHSWNTQTWCTQSWNNQAWNSPFYNCGEESLQSCMQFQPNSPASDLEAALEAAGEGLNVIQQTTRYFSTPQTMDLFLNYSMNMQPEDVESGGGGSPGRRRRRRRRRRR

SEQ ID NO: 29 - protein, 396 aa, Artificial Sequence
  transducible material
MGSSHHHHHHSSGLVPRGSHMTKSYSESGLMGEPQPQGPPSWTDECLSSQDEEHEADKKEDDLEAMNAEEDSLRNGGEEEDEDEDLEEEEEEEEEDDDQKPKRRGPKKKKMTKARLERFKLRRMKANARERNRMHGLNAALDNLRKVVPCYSKTQKLSKIETLRLAKNYIWALSEILRSGKSPDLVSFVQTLCKGLSQPTTNLVAGCLQLNPRTFLPEQNQDMPPHLPTASASFPVHPYSYQSPGLPSPPYGTMDSSHVFHVKPPPHAYSAALEPFFESPLTDCTSPSFDGPLSPPLSINGNFSFKHEPSAEFEKNYAFTMHYPAATLAGAQSHGSIFSGTAAPRCEIPIDNIMSFDSHSHHERVMSAQLNAIFHDESGGGGSPGRRRRRRRRRRR

SEQ ID NO: 30 - protein, 254 aa, Artificial Sequence [sequence omitted]

SEQ ID NO: 31 - protein, 323 aa, Artificial Sequence
  transducible material
MGSSHHHHHHSSGLVPRGSHMNGEEQYYAATQLYKDPCAFQRGPAPEFSASPPACLYMGRQPPPPPPHPFPGALGALEQGSPPDISPYEVPPLADDPAVAHLHHHLPAQLALPHPPAGPFPEGAEPGVLEEPNRVQLPFPWMKSTKAHAWKGQWAGGAYAAEPEENKRTRTAYTRAQLLELEKEFLFNKYISRPRRVELAVMLNLTERHIKIWFQNRRMKWKKEEDKKRGGGTAVGGGGVAEPEQDCAVTSGEELLALPPPPPPGGAVPPAAPVAAREGRLPPGLSASPQPSSVAPRRPQEPRESGGGGSPGRRRRRRRRRRR

SEQ ID NO: 32 - protein, 392 aa, Artificial Sequence
  transducible material
MGSSHHHHHHSSGLVPRGSHMAAELAMGAELPSSPLAIEYVNDFDLMKFEVKKEPPEAERFCHRLPPGSLSSTPLSTPCSSVPSSPSFCAPSPGTGGGGGAGGGGGSSQAGGAPGPPSGGPGAVGGTSGKPALEDLYWMSGYQHHLNPEALNLTPEDAVEALIGSGHHGAHHGAHHPAAAAAYEAFRGPGFAGGGGADDMGAGHHHGAHHAAHHHHAAHHHHHHHHHGGAGHGGGAGHHVRLEERFSDDQLVSMSVRELNRQLRGFSKEEVIRLKQKRRTLKNRGYAQSCRFKRVQQRHILESEKCQLQSQVEQLKLEVGRLAKERDLYKEKYEKLAGRGGPGSAGGAGFPREPSPPQAGPGGAKGTADFFLESGGGGSPGRRRRRRRRRRR

SEQ ID NO: 33 - protein, 471 aa, Artificial Sequence
  transducible material
MGSSHHHHHHSSGLVPRGSHMPNPRPGKPSAPSLALGPSPGASPSWRAAPKASDLLGARGPGGTFQGRDLRGGAHASSSSLNPMPPSQLQLPTLPLVMVAPSGARLGPLPHLQALLQDRPHFMHQLSTVDAHARTPVLQVHPLESPAMISLTPPTTATGVFSLKARPGLPPGINVASLEWVSREPALLCTFPNPSAPRKDSTLSAVPQSSYPLLANGVCKWPGCEKVFEEPEDFLKHCQADHLLDEKGRAQCLLQREMVQSLEQQLVLEKEKLSAMQAHLAGKMALTKASSVASSDKGSCCIVAAGSQGPVVPAWSGPREAPDSLFAVRRHLWGSHGNSTFPEFLHNMDYFKFHNMRPPFTYATLIRWAILEAPEKQRTLNEIYHWFTRMFAFFRNHPATWKNAIRHNLSLHKCFVRVESEKGAVWTVDELEFRKKRSQRPSRCSNPTPGPESGGGGSPGRRRRRRRRRRR

SEQ ID NO: 34 - protein, 11 aa, Human immunodeficiency virus type 1
YGRKKRRQRRR

SEQ ID NO: 35 - protein, 10 aa, Artificial Sequence
  protein transducible domain
RRRRRRRRRR

SEQ ID NO: 36 - protein, 11 aa, Artificial Sequence
  protein transducible domain
RRRRRRRRRRR

SEQ ID NO: 37 - protein, 20 aa, Artificial Sequence
  protein transducible domain
ESGGGGSPGRRRRRRRRRRR

SEQ ID NO: 38 - protein, 16 aa, Drosophila melanogaster
RQIKIWFQNRRMKWKK

SEQ ID NO: 39 - protein, 34 aa, herpes simplex virus 7
DAATATRGRSAASRPTQRPRAPARSASRPRRPVQ

SEQ ID NO: 40 - protein, 27 aa, Artificial Sequence
  protein transducible domain
GWTLNSAGYLLGKINLKALAALAKKIL

SEQ ID NO: 41 - protein, 18 aa, Artificial Sequence
  protein transducible domain
KLALKLALKALKAALKLA

SEQ ID NO: 42 - protein, 15 aa, Artificial Sequence
  protein transducible domain
AAVALLPAVLLALLP

SEQ ID NO: 43 - protein, 21 aa, Artificial Sequence
  protein transducible domain
KETWWETWWTEWSQPKKKRKV

SEQ ID NO: 44 - protein, 14 aa, Artificial Sequence
  protein transducible domain
RRWRRWWRRWWRRW

SEQ ID NO: 45 - protein, 12 aa, Artificial Sequence
  cell-targeting peptide
TSPLNIHNGQKL

SEQ ID NO: 46 - protein, 5 aa, Artificial Sequence
  cell-targeting peptide
CGKRK

SEQ ID NO: 47 - protein, 9 aa, Artificial Sequence
  cell-targeting peptide
CGNKRTRGC

SEQ ID NO: 48 - protein, 7 aa, Artificial Sequence
  cell-targeting peptide
SMSIARL

SEQ ID NO: 49 - protein, 7 aa, Artificial Sequence
  cell-targeting peptide
FQHPSFI

SEQ ID NO: 50 - protein, 7 aa, Artificial Sequence
  cell-targeting peptide
VHSPNKK

SEQ ID NO: 51 - protein, 6 aa, Artificial Sequence
  cell-targeting peptide
RRPYIL

SEQ ID NO: 52 - protein, 12 aa, Artificial Sequence
  cell-targeting peptide
EDYELMDLLAYL

SEQ ID NO: 53 - protein, 7 aa, Artificial Sequence
  cell-targeting peptide
LTVSPWY

SEQ ID NO: 54 - protein, 7 aa, Artificial Sequence
  cell-targeting peptide
ATWLPPR

SEQ ID NO: 55 - protein, 9 aa, Artificial Sequence
  linker
ESGGGGSPG

SEQ ID NO: 56 - protein, 16 aa, Homo sapiens
RHIKIWFQNRRMKWKK

SEQ ID NO: 57 - protein, 34 aa, Homo sapiens
KPKRRGPKKKKMTKARLERFKLRRMKANARERNR

SEQ ID NO: 58 - protein, 10 aa, Homo sapiens
EHWSYGLRPG

SEQ ID NO: 59 - protein, 20 aa, Artificial Sequence
  protein purification
MGSSHHHHHHSSGLVPRGSH

SEQ ID NO: 60 - protein, 17 aa, Human immunodeficiency virus type 1
TRQARRNRRRRWRERQR

SEQ ID NO: 61 - protein, 15 aa, flock house virus
RRRRNRTRRNRRRVR

SEQ ID NO: 62 - protein, 26 aa, Homo sapiens
KRRIRRERNKMAAAKSRNRRRELTDT

SEQ ID NO: 63 - protein, 28 aa, Homo sapiens
RIKAERKRMRNRIAASKSRKRKLERIAR

SEQ ID NO: 64 - protein, 22 aa, Artificial Sequence
  yease GCN4 peptide
KRARNTEAARRSRARKLQRMKQ

SEQ ID NO: 65 - protein, 20 aa, Influenza virus
GLFGAIAGFIENGWEGMIDG

SEQ ID NO: 66 - protein, 20 aa, Influenza virus
GDIMGEWGNEIFGAIAGFLG

SEQ ID NO: 67 - protein, 238 aa, Artificial Sequence
  Supercharged GFP
ASKGERLFRGKVPILVELKGDVNGHKFSVRGKGKGDATRGKLTLKFICTTGKLPVPWPTLVTTLTYGVQCFSRYPKHMKRHDFFKSAMPKGYVQERTISFKKDGKYKTRAEVKFEGRTLVNRIKLKGRDFKEKGNILGHKLRYNFNSHKVYITADKRKNGIKAKFKIRHNVKDGSVQLADHYQQNTPIGRGPVLLPRNHYLSTRSKLSKDPKEKRDHMVLLEFVTAAGIKHGRDERYK

SEQ ID NO: 68 - protein, 781 aa, Artificial Sequence
  scSOX9
MASKGERLFRGKVPILVELKGDVNGHKFSVRGKGKGDATRGKLTLKFICTTGKLPVPWPTLVTTLTYGVQCFSRYPKHMKRHDFFKSAMPKGYVQERTISFKKDGKYKTRAEVKFEGRTLVNRIKLKGRDFKEKGNILGHKLRYNFNSHKVYITADKRKNGIKAKFKIRHNVKDGSVQLADHYQQNTPIGRGPVLLPRNHYLSTRSKLSKDPKEKRDHMVLLEFVTAAGIKHGRDERYKGGSGGSGGSGGSHMNLLDPFMKMTDEQEKGLSGAPSPTMSEDSAGSPCPSGSGSDTENTRPQENTFPKGEPDLKKESEEDKFPVCIREAVSQVLKGYDWTLVPMPVRVNGSSKNKPHVKRPMNAFMVWAQAARRKLADQYPHLHNAELSKTLGKLWRLLNESEKRPFVEEAERLRVQHKKDHPDYKYQPRRRKSVKNGQAEAEEATEQTHISPNAIFKALQADSPHSSSGMSEVHSPGEHSGQSQGPPTPPTTPKTDVQPGKADLKREGRPLPEGGRQPPIDFRDVDIGELSSDVISNIETFDVNEFDQYLPPNGHPGVPATHGQVTYTGSYGISSTAATPASAGHVWMSKQQAPPPPPQQPPQAPPAPQAPPQPQAAPPQQPAAPPQQPQAHTLTTLSSEPGQSQRTHIKTEQLSPSHYSEQQQHSPQQIAYSPFNLPHYSPSYPPITRSQYDYTDHQNSSSYYSHAAGQGTGLYSTFTYMNPAQRPMYTPIADTSGVPSIPQTHSPQHWEQPVYTQLTRPESGGGGSPGRRRRRRRRRRR

(19) United States
(12) Patent Application Publication
(10) Pub. No.: US 2014/0289882 A1
(43) Pub. Date: Sep. 25, 2014

(54) COMPOSITIONS AND METHODS FOR RE-PROGRAMMING CELLS WITHOUT GENETIC MODIFICATION FOR REPAIRING CARTILAGE DAMAGE

(71) Applicants: Yong Zhu, Anaheim, CA (US); Shili Wu, Edmond, OK (US); Jun Bao, Rowland Heights, CA (US); Cong-Qiu Chu, Portland, OR (US)

(72) Inventors: Yong Zhu, Anaheim, CA (US); Shili Wu, Edmond, OK (US); Jun Bao, Rowland Heights, CA (US); Cong-Qiu Chu, Portland, OR (US)

(73) Assignees: OREGON HEALTH AND SCIENCE UNIVERSITY, Portland, OR (US); VIVOSCRIPT, INC., Costa Mesa, CA (US)

(21) Appl. No.: 14/233,390

(22) PCT Filed: Jul. 19, 2012
(86) PCT No.: PCT/US2012/047495

§ 371 Date: Jun. 10, 2014

Related U.S. Application Data
(60) Provisional application No. 61/509,114, filed on Jul. 19, 2011.

(51) Int. Cl. C07K 17/06; C12N 5/074; G01N 33/50; A61K 47/48

(52) U.S. Cl. CPC C07K 17/06; A61K 47/48246; A61K 47/48315; C12N 5/0696; G01N 33/5073
USPC 800/21; 530/350; 514/17.1; 424/520; 424/93.7; 435/377; 435/410; 435/29; 435/7.21; 435/6.13

(57) ABSTRACT

The present inventions are directed to compositions and methods regarding the reprogramming of other cells (such as embryonic stem cells (ESCs), induced pluripotent stem cells (iPSCs), MSCs, fibroblasts, hematopoietic stem cells, endothelian stem cells, adipocytes, chondrocytes, osteoblasts, osteoclasts and endothelial cells) into chondrogenic cells without introducing exogenous genes to the samples. In particular, the present inventions are directed to transducible materials that are capable of transducing into the biological samples but are not genes or causing genetic modifications. The present inventions also are directed to methods of reprogramming the path of biological samples or treating diseases using the tranducible compositions thereof.

### PRIORITY CLAIM

The present application claims the benefit of U.S. Provisional Patent Application No. 61/509,114, filed on Jul. 19, 2011, which is incorporated herein by reference in its entirety.

### BACKGROUND OF THE INVENTION

Embryonic stem cells are capable of differentiating into many types of cells of the human body. The majority of somatic cells are terminally differentiated and were believed to lack the capability of changing to other types of somatic cells. Recent advances in induced pluripotent stem cell (iPSC) and transdifferentiation fields have changed this paradigm. Somatic cells can be reprogrammed to induced pluripotent stem cell (iPSC), via the ectopic expression of four transcription factors: Oct4 (e.g. SEQ ID NO: 1), Sox2 (e.g. SEQ ID NO: 2), Klf4 (SEQ ID NO: 3), and cMyc (e.g. SEQ ID NO: 4) via viral transduction (Okita et al, Nature 448, 313-317 (2007); Takahashi and Yamanaka, Cell 126, 663-676 (2006)). A number of modified genetic approaches were further developed to produce iPSCs with potentially reduced risks, including using non-integrating adenoviruses to deliver reprogramming genes (Stadtfeld et al., Science 322, 945-949 (2008)), transient transfection of reprogramming plasmids (Okita et al., Science 322, 949-953 (2008)), a piggyac transposition system and Cre-excisable viruses (Soldner et al., Cell 136, 964-977 (2009); Woltjen et al., Nature 458, 766-770 (2009)). Furthermore, strategies of exploiting endogenous gene expression in certain cell types also allowed easier reprogramming and/or with less required exogenous genes (Aasen et al., Nat Biotechnol 26, 1276-1284 (2008); Kim et al., Nature 454, 646-650 (2008); Shi et al., Cell Stem Cell 2, 525-528 (2008)). Moreover, small molecules have been identified that enhance reprogramming efficiency and replace certain reprogramming factors (Huangfu et al., Nat Biotechnol 26, 795-797 (2008); Huangfu et al., Nat Biotechnol 26, 1269-1275 (2008); Li et al., Cell Stem Cell 4, 16-19 (2009); Shi et al., Cell Stem Cell 3, 568-574 (2008); Shi et al., Cell Stem Cell 2, 525-528 (2008)). However, all of those methods to date still involve the use of genetic materials that may introduce unknown, unwanted, or even harmful genome modifications in exogenous sequences in target cells. Further, these methods lack adequate control over the expression levels of transgenes. There is a need in the field to reprogram cells without relying upon or introducing exogenous genetic materials.

Articular cartilage is a highly organized tissue with low cell density and limited nutrient supply. Once it is damaged by trauma or degenerative arthritis, it has a limited capacity for regeneration. The most common joint disorder, osteoarthritis (OA), afflicts millions of people with symptoms that include severe pain, swelling, and clicking of joints. Surgical methods have been designed to repair cartilage result in the production of fibrocartilage which temporarily alleviates pain but is not a long-term solution becausefibrocartilage has poor resistance to shear forces. Hyaline cartilage, on the other hand, provides shock absorption and lubrication in diarthrodial joints as articular cartilage. Hyaline cartilage's extracellular matrix, comprises collagen fibrils composed of types II, IX, and XI collagen molecules, proteoglycans, and other matrix proteins and is substantially durable compared to fibrocartilage. Thus, the goal for repair of cartilage injury is to regenerate organized hyaline cartilage. Healing of cartilage damage with hyaline cartilage rather than fibrocartilage remains a challenging clinical problem.

One surgical procedure called microfracture involves drilling small holes into the subchondral bone marrow space underlying regions of damaged cartilage. This induces bleeding at the defect site and induces the formation of a blood clot. The clot contains pluripotent mesenchymal stem cells (MSCs) from the bone marrow, which have the potential to differentiate into condroblasts and condrocytes. However, like other surgical procedures, the cartilage formed is fibro-cartilaginous.

### BRIEF SUMMARY OF THE INVENTION

One aspect of the present disclosure relates to a transducible material for reprogramming a biological sample to a chondrogenic sample. The reprogrammed chondrogenic sample can repair cartilage injury or damage by regenerating or producing cartilage, preferably hyaline cartilage. The transducible material comprises an effector domain which is capable of exerting reprogramming changes of a biological sample once transduced into a biological sample. In certain embodiments, the effector domain is a polypeptide selected from the group consisting of Sox9, Sox6, Sox5, c-Myc, Klf4, Mef2C, Trps1, Gli3, Runx2, Dlx5, Dlx6, GATA-6, and Baf60c, and a homologous sequence thereof, and any combination thereof. In certain embodiments, the effector domain is inherently capable of transducing into the biological sample.

In certain embodiments, the transducible material further comprises a transduction domain which is covalently or non-covalently associated with or linked to the effector domain. In certain embodiments, the transduction domain is covalently linked to the effector domain through a linker.

In certain embodiments, the transducible material is capable of selectively transducing into one or more specific biological samples or becoming transducible in a specific environment surrounding the biological sample.

Another aspect of the present disclosure relates to a composition comprising a biological sample and a transducible material, wherein the transducible material has transduced into the biological material.

Another aspect of the present disclosure relates to a method of reprogramming a biological sample into a chondrogenic sample by exposing the biological sample to a composition comprising a transducible material.

Another aspect of the present disclosure relates to a method of treating a disease or condition involving cartilage damage in a biological organism comprising administering a pharmaceutical composition comprising a transducible material into the biological organism.

Another aspect of the present disclosure relates to a method of developing cell-based therapies for diseases or conditions involving cartilage damage comprising the step of reprogramming an iPSC, an embryonic stem cell, a progenitor cell or a differentiated cell to a transplantable chondrogenic cell using a transducible material.

Another aspect of the present disclosure relates to a method of developing disease models comprising the step of reprogramming an iPSC, an embryonic stem cell, a progenitor cell or a differentiated cell to a transplantable chondrogenic cell using a transducible material.

Another aspect of the present disclosure relates to a method of identifying an effector domain comprising covalently or non-covalently associating a test effector domain to a transduction domain to form a test transducible molecule, exposing the test molecule to a biological sample, and measuring a reprogramming level of the biological sample to a chondrogenic sample.

### BRIEF DESCRIPTION OF THE DRAWINGS

FIG. 1: Characterization of transducible materials (I). (A) Schematic of protein expression vector for transducible materials Oct4-11R (SEQ ID NO: 12), Sox2-11R (SEQ ID NO: 13), Klf4-11R (SEQ ID NO: 14), and cMyc-11R (SEQ ID NO: 15), Linker: SEQ ID NO: 55; Effector Domain: Oct4 (SEQ ID NO: 1), Sox2 (SEQ ID NO: 2), Klf4 (SEQ ID NO: 3), or cMyc (SEQ ID NO: 4). (B) Stability of the four transducible materials (Oct4-11R, Sox2-11R, Klf4-11R, and cMyc-11R) under the cell culture condition examined by Western blot analysis. (C) Schematic presentation of transducible materials containing super-charged green fluorescent protein (scGFP) fused to a transcription factor (TF).

FIG. 2: Characterization of transducible materials (II). Protein transduction of 11R-tagged transducible materials into 0G2-MEF cells examined by immunocytochemistry. Oct4: MEF cells transuded with Oct4-11R (green), Sox2: MEF cells transuded with Sox2-11R (red), Klf4: MEF cells transuded with Klf4-11R (red) and cMyc: MEF cells transuded with cMyc-11R (green). DAPI: Cells stained with DAPI to visualize the nuclei (blue) and the images were merged.

FIG. 3: Characterization of transducible materials (III). Protein induced pluripotent stem (piPS) cells clonally expanded and self-renewed in chemical defined media and feeder free condition.

FIG. 4: Generation of piPS cells by transducible materials Oct4-11R, Sox2-11R, Klf4-11R, and cMyc-11R. (A) Timeline of piPS cell generation. (B) Oct4-GFP⁺ piPS cell colonies initially observed around day 30-35. Phase: representative phase contrast image; and GFP: fluorescence image. (C) Oct4-GFP⁺ piPS cells sustained long term self-renewal under conventional mESC growth condition. (D) The long-term expanded piPS cells grew as compact and domed colonies that expressed strong ALP, a typical pluripotency marker. (E) piPS cells expressed other typical pluripotency markers, examined by immunofluorescence: SEA-1 (red), Sox2 (red), Oct4 (ed) and Nanog (red). DAPI: DAPI staining for visualization of the nuclei (blue), and the images were merged. (F) RT-PCR analysis of endogenous pluripotency gene expression in piPS cells. (G) Oct4 promoter methylation analysis by bisulfite genomic sequencing. Open and closed circles indicate unmethylated and methylated CpGs, respectively.

FIG. 5: In vitro and in vivo pluripotency of piPS cells (I). piPS cells effectively differentiated in vitro into cells in the three germ layers: Tuj1: characteristic TUJ1⁺ neuronal cells-ectoderm (red): Bryt: Brachyury⁺ mesoderm cells (red); and Sox17: Sox17⁺ definitive endoderm cells. Images were merged with DAPI (blue) staining.

FIG. 6: In vitro and in vivo pluripotency of piPS cells (II) (A) RT-PCR analysis of in vitro differentiation of piPS cells. (B) Chimeric embryos (13.5 dpc, 2 out of 7, left) obtained after transfer of the piPS cell aggregated embryos into a pseudo-pregnant mouse (top). Such piPS cells contributed to the germline cells (Oct4-GFP positive) in isolated genital ridge tissue from chimeric embryos (bottom).

FIG. 7: Schematic of protein expression vectors for transducible materials. His 6: SEQ ID NO: 59; Effector Domain: Ngn3 (SEQ ID NO: 8), PDX1 (SEQ ID NO: 9); MafA (SEQ ID NO: 10), or Foxp3 (SEQ ID NO: 11); Linker: SEQ ID NO: 55.

FIG. 8: Reprogramming of liver and pancreatic exocrine cells to insulin-producing beta cells by transducible materials His6-Ngn3-11R (SEQ ID NO: 30), His6-PDX1-11R (SEQ ID NO: 31) and His6-MafA-11R (SEQ ID NO: 32) in mouse (I). Mouse-1, Mouse-2 and Mouse-3 were treated with bovine serum albumin (BSA) protein (control group). Mouse-4, Mouse-5 and Mouse-6 were treated with His6-Ngn3-11R, His 6-PDX1-11R and His6-MafA-11R (treatment group). A) Immunofluorescent analysis (IFA) of Mouse-1 liver; B) IFA of Mouse-2 liver; and C) IFA of Mouse-3 liver.

FIG. 9: Reprogramming of liver and pancreatic exocrine cells to insulin-producing beta cells by transducible materials His6-Ngn3-11R, His6-PDX1-11R and His 6-MafA-11R in mouse (II). Mouse-1, Mouse-2 and Mouse-3 were treated with bovine serum albumin (BSA) protein (control group). Mouse-4, Mouse-5 and Mouse-6 were treated with His6-Ngn3-11R, His6-PDX1-11R and His6-MafA-11R (treatment group). A) IFA of Mouse-4 liver (1); B) IFA of Mouse-4 liver (2); C) IFA of Mouse-5 liver (1); D) IFA of Mouse-5 liver (2); E) IFA of Mouse-6 liver (1); and F) IFA of Mouse-6 liver (2).

FIG. 10: Reprogramming of liver and pancreatic exocrine cells to insulin-producing beta cells by transducible materials His6-Ngn3-11R, His6-PDX1-11R and His 6-MafA-11R in mouse (III). Mouse-1, Mouse-2 and Mouse-3 were treated with bovine serum albumin (BSA) protein (control group). Mouse-4, Mouse-5 and Mouse-6 were treated with His6-Ngn3-11R, His6-PDX1-11R and His6-MafA-11R (treatment group). A) IFA of Mouse-1 pancrease; B) IFA of Mouse-2 pancrease (1); C) IFA of Mouse-2 pancrease (2); and D) IFA of Mouse-3 pancrease.

FIG. 11: Reprogramming of liver and pancreatic exocrine cells to insulin-producing beta cells by transducible materials His6-Ngn3-11R, His6-PDX1-11R and His 6-MafA-11R in mouse (IV). Mouse-1, Mouse-2 and Mouse-3 were treated with bovine serum albumin (BSA) protein (control group). Mouse-4, Mouse-5 and Mouse-6 were treated with His6-Ngn3-11R, His6-PDX1-11R and His6-MafA-11R (treatment group). A) IFA of Mouse-4 pancrease (1); B) IFA of Mouse-4 pancrease (2); C) IFA of Mouse-5 pancrease (1); D) IFA of Mouse-5 pancrease (2); and E) IFA of Mouse-6 pancrease.

FIG. 12: Reprogramming of T cells to Treg cells by transducible material His 6-Foxp3-11R (SEQ ID NO: 33) (IA). Flow cytometric analysis of CD4 and CD25 protein expression in PBMC with lacking of CD14 monocytes: isotope control, PBS control, sample buffer control and protein (BSA 100 μg/ml) control.

FIG. 13: Reprogramming of T cells to Treg cells by transducible material His 6-Foxp3-11R (IB). Flow cytometric analysis of CD4 and CD25 protein expression in PBMC with lacking of CD14 monocytes treated with His16-Foxp3-11R of 10 μg/ml, 20 μg/ml, or 50 μg/ml.

FIG. 14: Reprogramming of T cells to Treg cells by transducible material His 6-Foxp3-11R (IIA). Flow cytometric analysis of CD4 and CD25 protein expression in PBMC: isotope control, and PBS control.

FIG. 15: Reprogramming of T cells to Treg cells by transducible material His 6-Foxp3-11R (IIB). Flow cytometric analysis of CD4 and CD25 protein expression in PBMC: sample buffer control and protein (BSA 100 μg/ml) control.

FIG. 16: Reprogramming of T cells to Treg cells by transducible material His 6-Foxp3-11R (IIC). Flow cytometric analysis of CD4 and CD25 protein expression in PBMC treated with His16-Foxp3-11R of 10 μg/ml, or 50 μg/ml.

FIG. 17: Reprogramming of T cells to Treg cells by transducible material His 6-Foxp3-11R (IID). Flow cytometric analysis of CD4 and CD25 protein expression in PBMC treated with His16-Foxp3-11R of 100 μg/ml.

FIG. 18: Sequences of the exemplary effector domains of Foxo1 and Foxo3.

FIG. 19: Sequences of the exemplary effector domains.

FIG. 20: Sequences of the exemplary transducible materials: Effector domain-11R.

FIG. 21: Sequences of the exemplary transducible materials: His6-Effector domain-11R.

FIG. 22: Sequences of the exemplary transducible materials: HA2-supercharged GFP-Effector domain (a-m), and scGFP-Sox9-11R (n).

FIG. 23: Penetration of scSox9 protein into human skin fibroblast cell line (HHF, A and B) and human bone marrow derived mesenchymal stem cells (MSC, C and D). Cells were incubated with 10 μg/ml of super charged green fluorescence protein (scGFP, A and C) or recombinant Sox9-scGFP fusion protein (scSox9, B and D).

FIG. 24: scSox9 induced increased proliferation of MSC. MSC were cultured in DMEM containing 10% FBS and low glucose (1.5 g/l) with addition of scSox9 or buffer only for 48 hours.

FIG. 25: scSox9 induced aggrecan expression and chondrogenesis, as shown by positive toluidine blue staining of MSC treated with scSox9 at 3 days (B), and MSC with scSox9 at 14 days (C), in contrast to negative toluidine blue staining of MSC with buffer (A).

FIG. 26: scSox9 induced collagen type II expression, as shown by positive collagen type II monoclonal antibody (immunoperoxidase) staining of MSC treated with scSox9 (B and D), in contrast to negative staining of MSC treated with scGFP (A and C).

FIG. 27: scSox9 induced increased collagen type II but decreased collagen type I and type X expression. At the indicated time point, RNA was extracted from MSCs treated with buffer only or 10 μg/ml of scSox9, and RT-PCR was performed for collagen (Col) type I, II and X mRNA expression. Expression as relative to GAPDH.

### DETAILED DESCRIPTION OF THE INVENTION

One aspect of the present disclosure relates to a transducible material for reprogramming a biological sample to a chondrogenic sample. The reprogrammed chondrogenic sample can repair cartilage injury or damage by regenerating or producing cartilage, preferably hyaline cartilage. The transducible material comprises an effector domain.

In certain embodiments, a transducible material used herein refers to a material or a molecule which is not DNA or derived from DNA but is capable of crossing or transducing or being crossed through a membrane of a biological sample (e.g., a cell membrane) so that the transducible material can enter or be brought into the inside of the biological sample from the outside of the biological sample and exerts reprogramming efforts. For example, the transducible material may interact with cell-surface receptors which facilitate the entry of the material into cells through receptor mediated endocytosis.

In certain embodiments, a transducible material is a selective transducible material which is more likely to transduce into a specific type of biological samples (e.g. cancer or tumor cells) or becomes transducible in a specific microenvironment in or around a biological sample (e.g. microenvironment around cancer or tumor) than other biological samples. For example, the selective transducible material comprises a transduction domain (e.g. a cell-targeting peptide or an activatable cell penetrating peptide) that preferably delivers the selective transducible material into a specific type of biological sample or become transducible in a microenvironment around a biological sample.

Without being bounded to any theories, it is contemplated that the transducible materials may cross a cell membrane and enter into cytoplasm to reprogram cytoplasm activities such as translation, post-translation modification, signaling pathway, or the apoptosis pathway. It is further contemplated that the transducible material may cross the nucleus membrane and reprogram or modulate DNA or chromosomal replication, gene transcription, and RNA splicing.

An effector domain is a motif or a molecule which, once inside a biological sample, is capable of exerting reprogramming changes of the biological sample. The effector domain may interact with molecules (e.g., proteins, DNA, RNA, sugars, and lipids) in the biological sample (e.g., in cytoplasm or nuclei) and lead to changes such as proliferation, differentiation, dedifferentiation, transdifferentiation, retrodifferentiation, transdetermination, apoptosis, and morphogenesis. The effector domain can be 1) a polypeptide, or a fragment or a mimic thereof; 2) a polynucleotide which cannot be gene expressed once transduced or incorporated into the genome of the biological sample or cause genetic modification but nevertheless interacts with molecules in the biological sample (e.g., a ribozyme, an antisense molecule, a siRNA or miRNA, an oligonucleotide, and the like); and 3) a small molecule or other chemical compound (e.g. chemotherapy drugs).

In certain embodiments, an effector domain is inherently transducible, e.g. PDX1 (e.g. SEQ ID NO: 9) and NeuroD (e.g. SEQ ID NO: 7).

One example of the effector domain is part or the entirety of a polypeptide such as a transcription factor, a chromosome remodeling protein, an antibody, or a fragment or mimic thereof. Another example of the effector domain is a small molecule which is not a polymer and binds with a biopolymer such as protein, nucleic acid, or polysaccharide and alters the activity or function of the biopolymer. Examples of small molecules include, without limitation, acetylation inhibitors, transcription activators, signal pathway activators, signal pathway inhibitors, and methylation inhibitors.

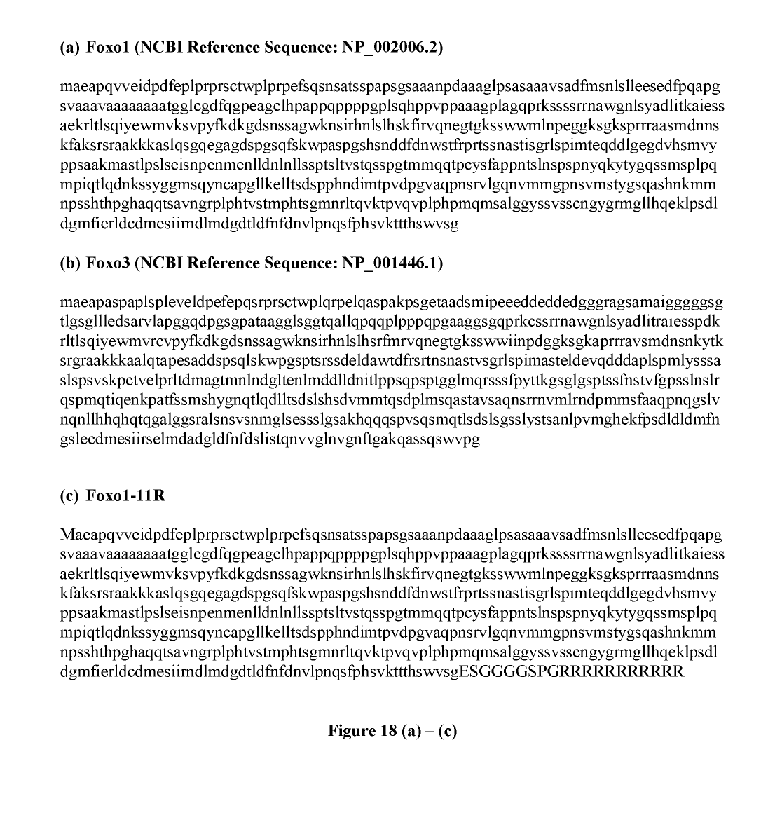

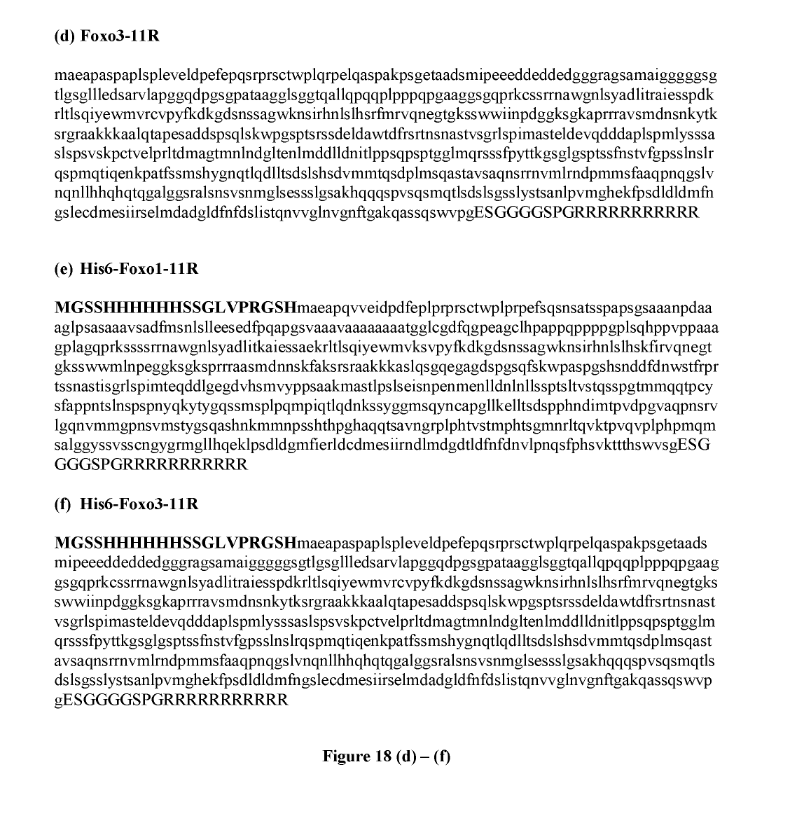

In another embodiment, an effector domain can be at least one polypeptide that reprograms a somatic or stem cell into a chondrogenic cell, reprograms a somatic cell into a stem cell or change a cell state from one to another. For example, the effector domain can be 1) a polypeptide selected from the group consisting of Klf4 (e.g. SEQ ID NO: 3), Sox2 (e.g. SEQ ID NO: 2), Lin28 (e.g. SEQ ID NO: 5), Oct4 (e.g. SEQ ID NO: 1), cMyc (e.g. SEQ ID NO: 4), Nanog (e.g. SEQ ID NO: 6), and any combination thereof; 2) a polypeptide selected from the group consisting of Klf4, Sox2, Oct4, cMyc, and any combination thereof; 3) a polypeptide selected from the group consisting of Sox2, Oct4, Lin28, Nanog, and any combination thereof; 4) a polypeptide selected from the group consisting of Ngn3 (e.g. SEQ ID NO: 8), PDX1 (e.g. SEQ ID NO: 9), MafA (e.g. SEQ ID NO: 10), NeuroD (e.g. SEQ ID NO: 7), and any combination thereof; 5) a polypeptide comprising Foxp3 (e.g. SEQ ID NO: 11), Foxo1(e.g. FIG. 18a) and Foxo3 (e.g. FIG. 18b); 6) a polypeptide selected from the group consisting of Oct4, Sox2, Klf4, Lin28, Nanog, cMyc, Ngn3, PDX1, MafA, NeuroD, Foxp3, Foxo1 and Foxo3, and any combination thereof; 7) a combination of polypeptides Oct4, Sox2, Klf4 and cMyc; 8) a combination of polypeptides Ngn3, PDX1 and MafA; 9) a polypeptide selected from the group consisting of Sox9, Sox6, Sox5, c-Myc, Klf4, Mef2C, Trps1, Gli3, Runx2, Dlx5, Dlx6, GATA-6 and Baf60c; 10) a polypeptide selected from the group consisting of Oct4, Sox2, Klf4, Lin28, Nanog, cMyc, Ngn3, PDX1, MafA, NeuroD, Foxp3, Foxo1, Foxo2, Sox9, Sox6, Sox5, c-Myc, Klf4, Mef2C, Trps1, Gli3, Runx2, Dlx5, Dlx6, GATA-6 and Baf60c, and any combination thereof; and 11) a first polypeptide selected from the group consisting of Sox9, Sox6, Sox5, c-Myc, Klf4, Mef2C, Trps1, Gli3, Runx2, Dlx5, Dlx6, GATA-6 and Baf60c, and any combination thereof; and a second polypeptide selected from the group consisting of Oct4, Sox2, Klf4, Lin28, Nanog, cMyc, Ngn3, PDX1, MafA, NeuroD, Foxp3, Foxo1 and Foxo3.

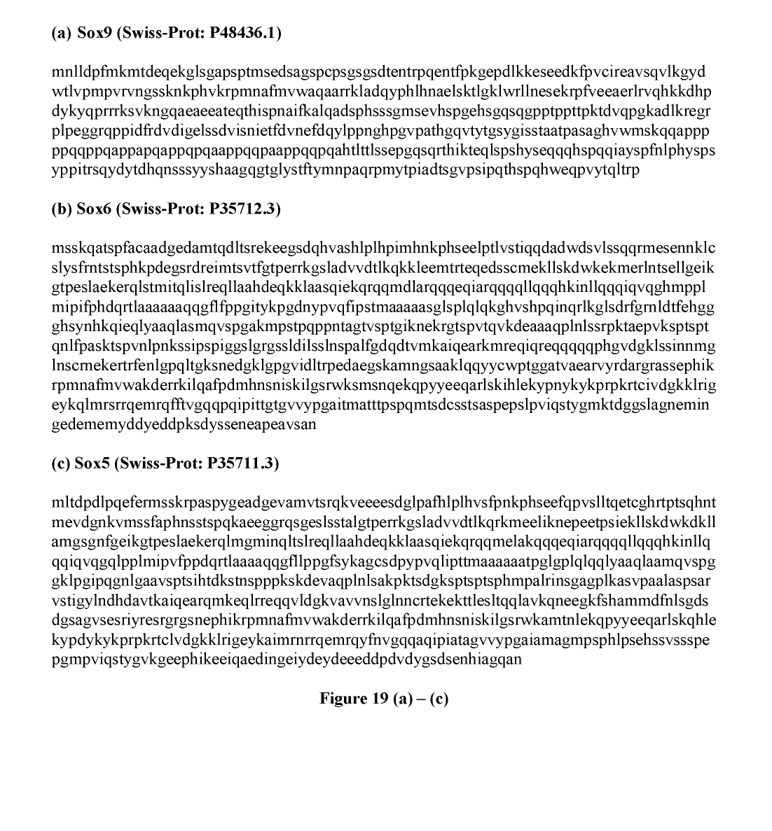

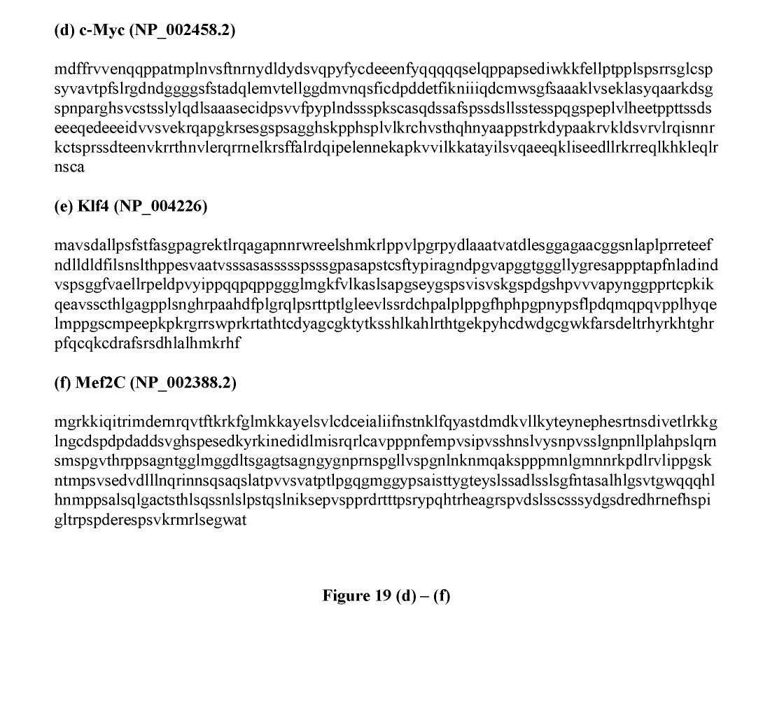

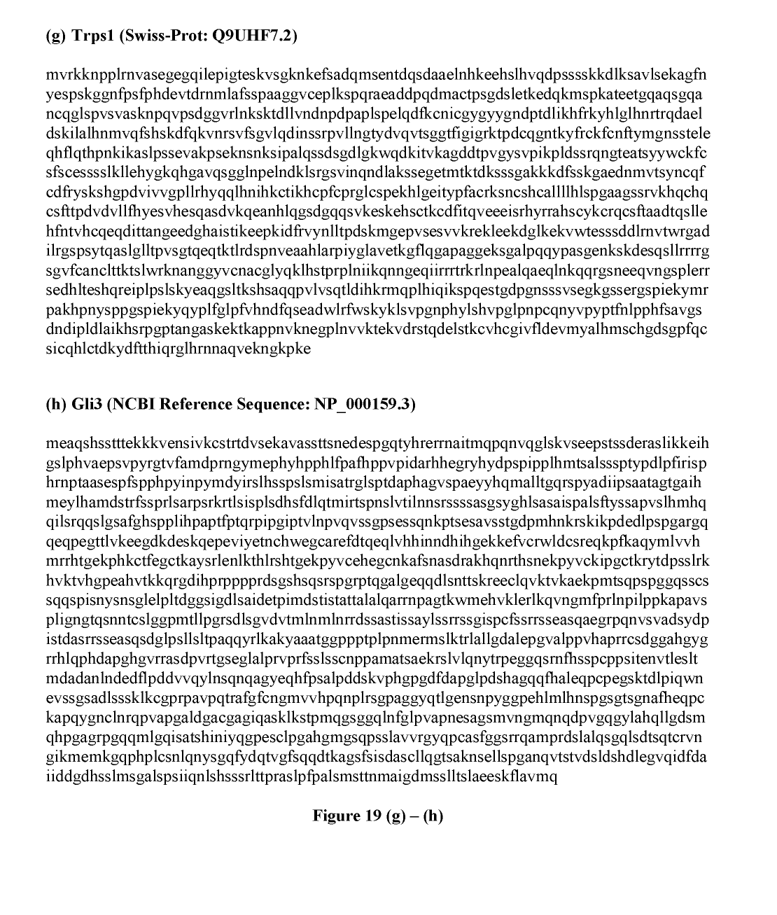

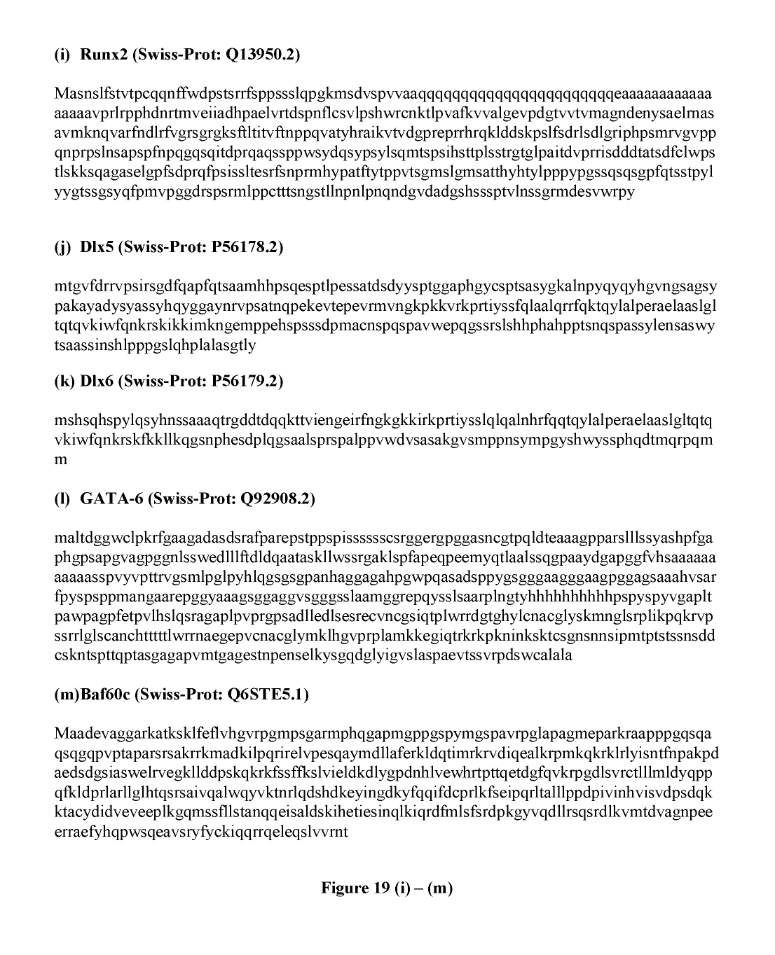

TABLE 1
Exemplary sequences of effector domains

| | Exemplary | Exemplary |
|---|---|---|
| | Accession Number | Polypeptide |
| Effector Domain | (Database) | Sequence |
| Sox9 | P48436.1 | FIG. 19 (a) |
| | (Swiss-Prot) | |
| Sox6 | P35712.3 | FIG. 19 (b) |
| | (Swiss-Prot) | |
| Sox5 | P35711.3 | FIG. 19 (c) |
| | (Swiss-Prot) | |
| c-Myc | SEQ ID NO: 4; | FIG. 19 (d) |
| | NP_002458.2 | |
| | (NCBI Reference | |
| | Sequence) | |
| Klf4 | SEQ ID NO: 3; | FIG. 19 (e) |
| | NP_004226 | |
| | (NCBI Reference | |
| | Sequence) | |
| Mef2C | NP_002388.2 | FIG. 19 (f) |
| | (NCBI reference) | |
| Trps1 | (Q9UHF7.2) | FIG. 19 (g) |
| | (Swiss-Prot) | |
| Gli3 | NP_000159.3 | FIG. 19 (h) |
| | (NCBI Reference | |
| | Sequence) | |
| Runx2 | Q13950.2 | FIG. 19 (i) |
| | (Swiss-Prot) | |
| Dlx5 | P56178.2 | FIG. 19 (j) |
| | (Swiss-Prot) | |
| Dlx6 | P56179.2 | FIG. 19 (k) |
| | (Swiss-Prot) | |
| GATA-6 | Q92908.2 | FIG. 19 (l) |
| | (Swiss-Prot) | |
| Baf60c | Q6STE5.1 | FIG. 19 (m) |
| | (Swiss-Prot) | |

The polypeptides provided herein encompass their homologous sequences. A “homologous sequence” as used herein, refers to a polypeptide which shares sufficient percentage of identity in amino acid sequence with a reference polypeptide to which it is homologous. In certain embodiments, a homologous sequence shares at least 70%, at least 75%, at least 80%, at least 85%, at least 88%, at least 90%, at least 93%, at least 95%, at least 97%, at least 98%, or at least 99% of identity in amino acid sequence with one of the polypeptides provided herein useful as effector domain. The polypeptides having homologous sequences have substantially the same activity as the effector domains disclosed herein.

The percentage of identity in amino acid sequence can be determined as the percentage of amino acid residues in a candidate amino acid sequence that are identical to the amino acid residues in a reference amino acid sequence, after aligning the sequences and, if necessary, introducing gaps, to achieve the maximum number of identical amino acids. Conservative substitution of the amino acid residues may be considered or may not considered as identical residues. “Conservative substitution” as used herein refers to replacing an amino acid residue with another amino acid residue that has similar physiochemical properties (e.g., hydrophobicity and molecular bulk of the side chain).

Alignment for purposes of determining percent amino acid sequence identity can be achieved in various ways known in the art. For instance, alignment of amino acid sequences may be made using publicly available tools such as BLASTp (available on the website of U.S. National Center for Biotechnology Information (NCBI): http://blast.ncbi.nlm.nih.gov/Blast.cgi, see also, Altschul S. F. et al, J. Mol. Biol., 215:403-410 (1990); Stephen F. et al, Nucleic Acids Res., 25:3389-3402 (1997)), ClustalW2 (available on the website of European Bioinformatics Institute: http://www.ebi.ac.uk/Tools/msa/clustalw2/, see also, Higgins D. G. et al, Methods in Enzymology, 266:383-402 (1996); Larkin M. A. et al, Bioinformatics (Oxford, England), 23(21): 2947-8 (2007)), and TCoffee (available on the website of Swiss Institute of Bioinformatics, see also, Poirot O. et al, Nucleic Acids Res., 31(13): 3503-6 (2003); Notredame C. et al, J. Mol. Boil., 302(1): 205-17 (2000)). Those skilled in the art may use the default parameters provided by the tool, or may customize the parameters as appropriate for the alignment, such as for example, by selecting a suitable algorithm.

The polypeptides provided herein further encompass their functional equivalents. A “functional equivalent” as used herein, refers to a polypeptide which possesses functional or structural characteristics that are substantially similar to all or part of a parent polypeptide. The polypeptides provided herein encompass their functional equivalents that can exert a substantially similar function, i.e. reprograming a somatic cell into a stem cell or changing a cell state from one to another. A functional equivalent of the polypeptides provided herein (i.e. parent polypeptides) may be a fragment, mutant, derivative, variant, or analog of a parent polypeptide, and may contain chemical or biological modifications. The functional equivalent may have one or more amino acid substitutions, additions, deletions, insertions, truncations, modifications (e.g. phosphorylation, glycosylation, labeling, etc.), or any combination thereof, of the parent polypeptide. The functional equivalent may include naturally occurring variants of the parent polypeptide and artificial polypeptide sequences such as those obtained by recombinant methods or chemical synthesis. The functional equivalent may contain non-naturally occurring amino acid residues.

In certain embodiments, a transducible material further comprises a transduction domain. A transduction domain is a motif that is capable of facilitating the entry of the transducible material into a biological sample (e.g., a cell). The transducible domain is associated with the effector domain covalently, noncovalently or via a linker. In certain embodiments, the transduction domain is covalently linked to the effector domain through a linker. In certain embodiments, the linker is a glycine-rich linker that comprises one or more glycine residues (e.g. esggggspg (SEQ ID NO: 55)).

Examples of a transduction domain include, without limitation, polymers such as cationic lipid polymers and nanoparticles, protein transduction domains (PTD), cell penetrating peptides (CPP1), cell permeating peptides (CPP2), activatable cell penetrating peptides or conjugates (ACPP), cell-targeting peptides (CTP), and supercharged proteins.

CPP1, CPP2, PTD and super charged proteins are peptides known to facilitate delivery of a molecular cargo associated thereof into cells. The association between a CPP1, CPP2 PTD or super charged proteins and the molecular cargo can be through covalent bond or non-covalent interactions. The molecular cargo can be small chemical molecules, peptides, protein, fragment of DNA, RNA such as siRNA and miRNA, or nanosize particles. For example, CPP1 and PTD include 5 to 20 amino acid peptide motifs that are capable of penetrating cells independent of surface transporters and of cell cycle phase. CPP1 and PTD can also be capable of penetrating through blood-brain barriers. CPP1 and PTD can deliver proteins and peptides in vitro and in vivo with uniform distribution throughout the organism after parenteral administration. Cationic PTDs can act as nuclear localization signals and carry an associated molecular cargo to cell nuclei. Examples of protein transduction domains include, without limitation, TAT (e.g. YGRKKRRQRRR, SEQ ID NO: 34), poly-arginine (e.g. poly-arginine having 7-11 arginine residues such as RRRRRRR, RRRRRRRR, RRRRRRRRR, RRRRRRRRRR (SEQ ID NO: 35) and RRRRRRRRRRR (SEQ ID NO: 36)), Penetratin (Antennapedia, e.g. RQIKIWFQNRRMKWKK (SEQ ID NO: 38)), VP22 (e.g. DAATATRGRSAASRPTQRPRAPARSASRPRRPVQ (SEQ ID NO: 39)), Transportan (e.g. GWTLNSAGYLLGKINLKALAALAKKIL (SEQ ID NO: 40)), MAP (e.g. KLALKLALKALKAALKLA (SEQ ID NO: 41)), MTS (e.g. AAVALLPAVLLALLP (SEQ ID NO: 42)), PEP-1 (e.g. KETWWETWWTEWSQPKKKRKV (SEQ ID NO: 43)), Arg/Trp analogue (e.g. RRWRRWWRRWWRRW (SEQ ID NO: 44)), polyguanidine peptoids (e.g. polyguanidine peptoids with a 6-methylene spacer between backbone and guanidine group such as N-arg 5, 7 or 9 peptoids), inherent protein transduction domain, (e.g. RHIKIWFQNRRMKWKK (SEQ ID NO: 56), KPKRRGPKKKKMTKARLERFKLRRMKANARERNR (SEQ ID NO: 57), HIV-1 Rev (e.g. TRQARRNRRRRWRERQR (SEQ ID NO: 60)), Flock house virus coat peptide (e.g. RRRRNRTRRNRRRVR (SEQ ID NO: 61)), DNA-binding peptides such as c-Fos (e.g. KRRIRRERNKMAAAKSRNRRRELTDT (SEQ ID NO: 62)), c-Jun (e.g. RIKAERKRMRNRIAASKSRKRKLERIAR (SEQ ID NO: 63)), yeast GCN4 (e.g. KRARNTEAARRSRARKLQRMKQ (SEQ ID NO: 64)), Fusogenic HA2 peptide (e.g. GLFGAIAGFIENGWEGMIDG (SEQ ID NO: 65), GDIMGEWGNEIFGAIAGFLG (SEQ ID NO: 66)).

A supercharged protein can be a peptide or protein modified to have an increased or decreased overall net charge (either positive or negative net charge) relative to the unmodified form of the protein. In certain embodiments, superpositively charged protein, which has an overall net positive charge, can be used as the transduction domain. Examples of the supercharged proteins include, without limitation, supercharged GFP (e.g. ASKGERLFRGKVPILVELKGDVNGHKFSVRGKGKGDATRGKLTLKFICTTGKLP VPWPTLVTTLTYGVQCFSRYPKHMKRHDFFKSAMPKGYVQERTISFKKDGKYK TRAEVKFEGRTLVNRIKLKGRDFKEKGNILGHKLRYNFNSHKVYITADKRKNGI KAKFKIRHNVKDGSVQLADHYQQNTPIGRGPVLLPRNHYLSTRSKLSKDPKEKR DHMVLLEFVTAAGIKHGRDERYK (SEQ ID NO: 67)), and other supercharged proteins disclosed in U.S. patent application US20110112040, which is incorporated herein by reference in its entirety. Naturally occurring supercharged proteins can also be used, such as HBEGF (Accession No.: Q99075), N-DEK (Accession No.: P35659), C-jun (Accession No.: P05412), and HGF (Accession No.: P14210), and other naturally supercharged proteins as disclosed in U.S. patent application US20110112040, which is incorporated herein by reference in its entirety.

Cell-targeting peptides are proteins or peptides that bind to cell-surface receptors and enter cells through endocytosis. In certain embodiments, a cell-target peptide targets specific tissues or cell types, for example, GnRH peptides (e.g. SEQ ID NO: 58) target biological samples that express GnRH receptors (e.g. solid tumors and hormone-responsive cancer cell lines). More examples of cell-targeting peptides and the specific biological samples targeted are listed in Table 2.

TABLE 2
Examples of cell-targeting peptides and the
specific biological samples targeted.

| | | | Targeted tissue or |
|---|---|---|---|
| Sequence ID | Peptide | | cells and the |
| No. | sequence | Length | cellular targets |
| SEQ ID NO: 45 | TSPLNIHN | 12 | Human head and |
| | GQKL | | neck solid tumors |
| SEQ ID NO: 46 | CGKRK | 5 | Tumor neovascula- |
| | | | ture, Heparan |
| | | | sulfate |
| SEQ ID NO: 47 | CGNKRTRGC | 7 | breast carcinoma |
| SEQ ID NO: 48 | SMSIARL | 7 | Prostate vascula- |
| | | | ture |
| SEQ ID NO: 49 | FQHPSFI | 7 | hepatocellular |
| | | | carcinoma cell |
| | | | line |
| | RGD | 3 | Integrin recep- |
| | | | tor′, αVβ3 |
| | NGR | 3 | Tumor neovascula- |
| | | | ture, Amino- |
| | | | peptidase N |
| SEQ ID NO: 50 | VHSPNKK | 7 | Endothelial |
| | | | VCAM-1 expressing |
| | | | cells; VCAM-1 |
| SEQ ID NO: 51 | RRPYIL | 6 | Adenocarcinoma |
| | | | cells; |
| | | | Neurotensin |
| | | | receptor |
| SEQ ID NO: 52 | EDYELMDL | 12 | Various carcinoma |
| | LAYL | | |
| SEQ ID NO: 53 | LTVSPWY | 7 | breast carcinoma; |
| | | | erbB2 |
| SEQ ID NO: 54 | ATWLPPR | 7 | Tumor neovascula- |
| | | | ture; VEGF |
| | | | receptor |

An activatable cell penetrating peptide or conjugate (ACPP) comprises a cationic CPP1, CPP2, PTD or super charged proteins and a netutralizing anionic counterpart. In certain embodiments, the cationic CPP1, CPP2, PTD or super charged proteins and the anionic counterpart are associated via noncovalent interactions (e.g. charge-charge interaction) and/or covalent cleavable linker (e.g. matrix metaloprotease (MMP) cleavable sequence). Transduction of an ACPP into cells are inhibited until the noncovalent interactions are disrupted and/or the cleavable linker is cleaved. For example, without being bound to any theory, the anionic counterparts comprise one or more pH sensitive groups such as sulfonamide groups, which are protonated at pH 7.4 (the pH in the blood stream) and become neutral at slightly acidic pH (e.g. pH 6.8). Therefore, charge-charge interactions between cationic CPP1, CPP2, PTD or super charged proteins and the anionic counterpart can be interrupted in slightly acidic microenvironment (e.g. in or around tumor or cancer). MMP concentration in blood stream is lower than that in a microenvironment around tumor or cancer. Therefore, MMP cleavable sequence, which is not cleaved in the bloodstream, is cleaved in environment around tumor or cancer. The cationic CPP1, CPP2, PTD or super charged proteins is no longer neutralized by the anionic counterpart, and therefore is exposed to facilitate the translocation into cells (e.g. tumor or cancer cells). In certain embodiments, the CPP1, CPP2 or PTD is TAT. In certain embodiments, the anionic counterparts comprise pH-sensitive polymer (e.g. di-block copolymer) comprising pH-sensitive groups (e.g. sulfonamide groups).

For another example, an activatable cell penetrating conjugate comprises a conventional hydrophobic core made of a polymer into which an effective domain is incorporated, a peripheral hydrophilic layer composed of poly-ethylene glycol and one or more cationic CPP1s, CPP2s, PTDs or super charged proteins, and one or more anionic counterpart that neutralize the cationic CPP12, CPP2s, PTDs or super charged proteins through charge-charge interactions. Such charge-charge interactions are expected to shield the cationic charges during delivery until the transduction material reaches a slightly acidic microenvironment (e.g. tumor or cancer), which triggers protonation of the anionic counterparts and disrupts the charge-charge association. Subsequently the cationic CPP1s, CPP2s, PTDs or super charged proteins, previously quenched by the anionic counterpart, are now capable of facilitating delivery of the effector domain into the surrounding cells (e.g. tumor or cancer cells).

In certain embodiments, a selective transducible material comprises a transduction domain selected from the group consisting of cell-targeting peptides and activatable cell penetrating peptides and activatable cell penetrating conjugates.

A transduction domain is associated to an effector domain via covalent bond, non-covalent interactions or through a linker. Thus a transducible material can be made by obtaining the transduction domain and the effector domain separately and associate together through a covalent bond or non-covalent interactions (e.g., repulsive interactions, dipole interactions, hydrogen bonding interactions, dispersive interactions, charge-charge interactions, solvent, counter ion and entropic effects, and water and hydrophobic effects). In certain embodiments, the transduction material is prepared by mixing the effector domain and transducible domain. Alternatively, a transducible material can be produced by isolating the material from natural resources or recombinantly. In the case when both domains are peptides or polypeptides, the effector domain can be linked to the N-terminus or C-terminus of the transduction domain and the transducible polypeptide can be made synthetically through chemical synthesis or recombinantly through recombinant technology.

In certain embodiments, a transducible material comprises an effector domain that is inherently transducible, and a transduction domain associated with the effector domain via covalent or non-covalent interactions.

In certain embodiments, a transducible material further comprises one or more motifs that do not interrupt the function of the effector domain or the transduction domain. In certain embodiments, these motifs are linked covalently, non-covalently or through a linker to the effector domain and/or the transduction domain. In certain embodiments, these motifs facilitate the preparation and/or purification of the transducible material. One example of such motif is a polyhistidine-tag to facilitate protein purification in preparation of the transducible material. In certain embodiments, the polyhistidine-tag comprises at least six histidine residues (e.g. MGSSHHHHHHSSGLVPRGSH (“His6,” SEQ ID NO: 59)).

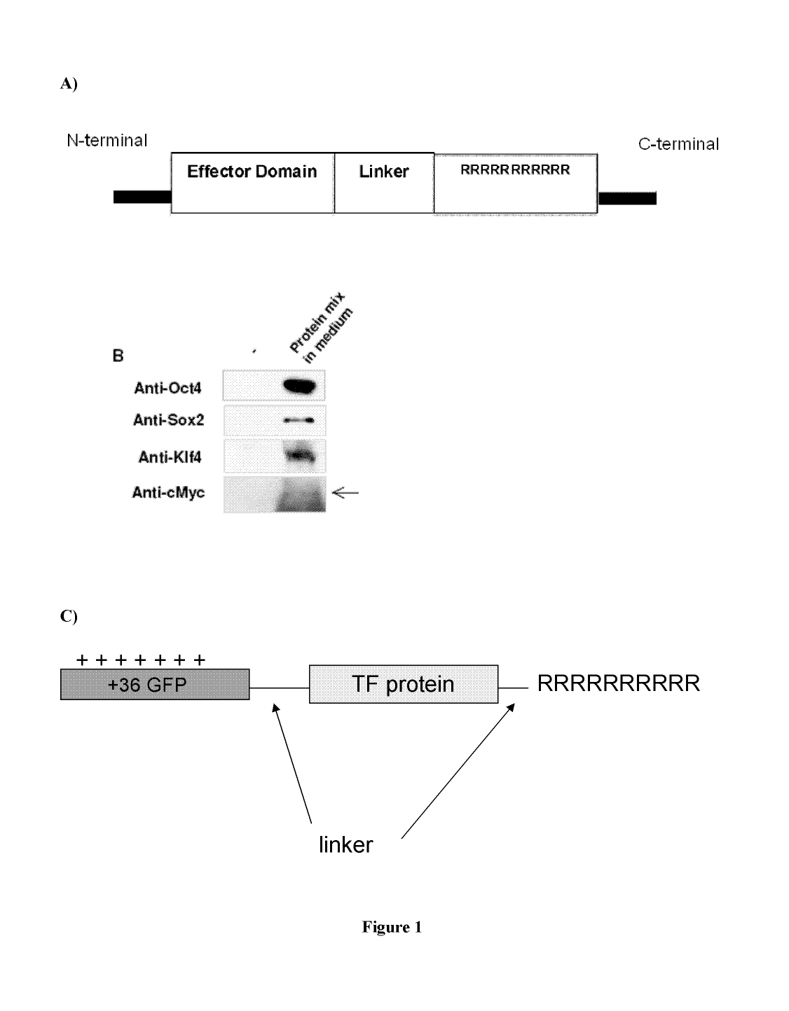

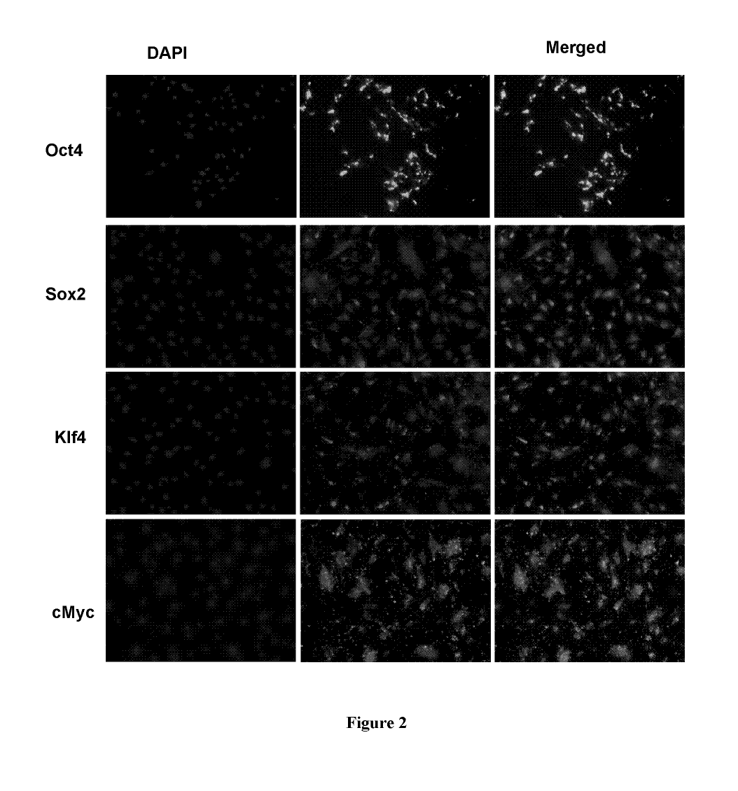

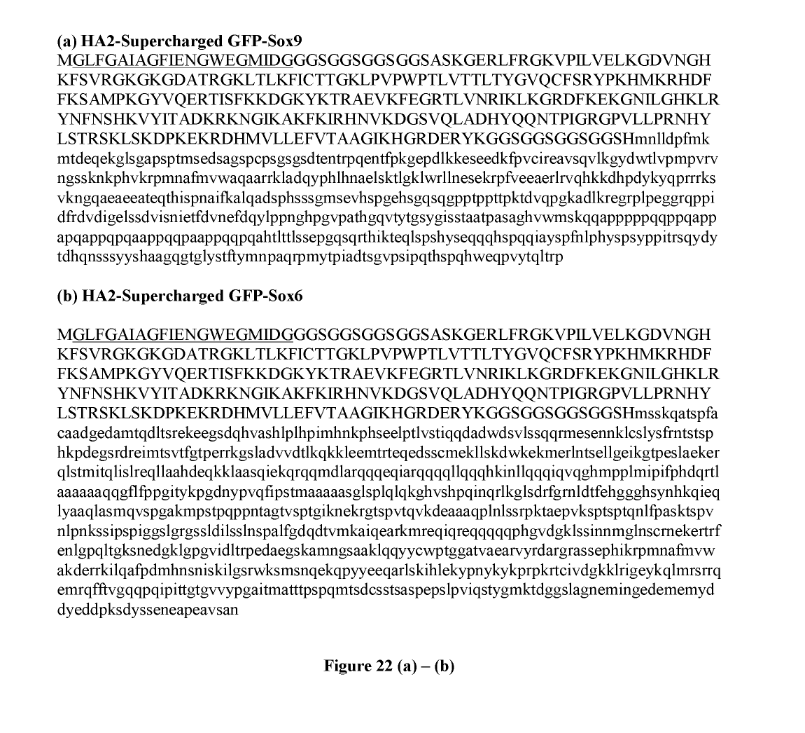

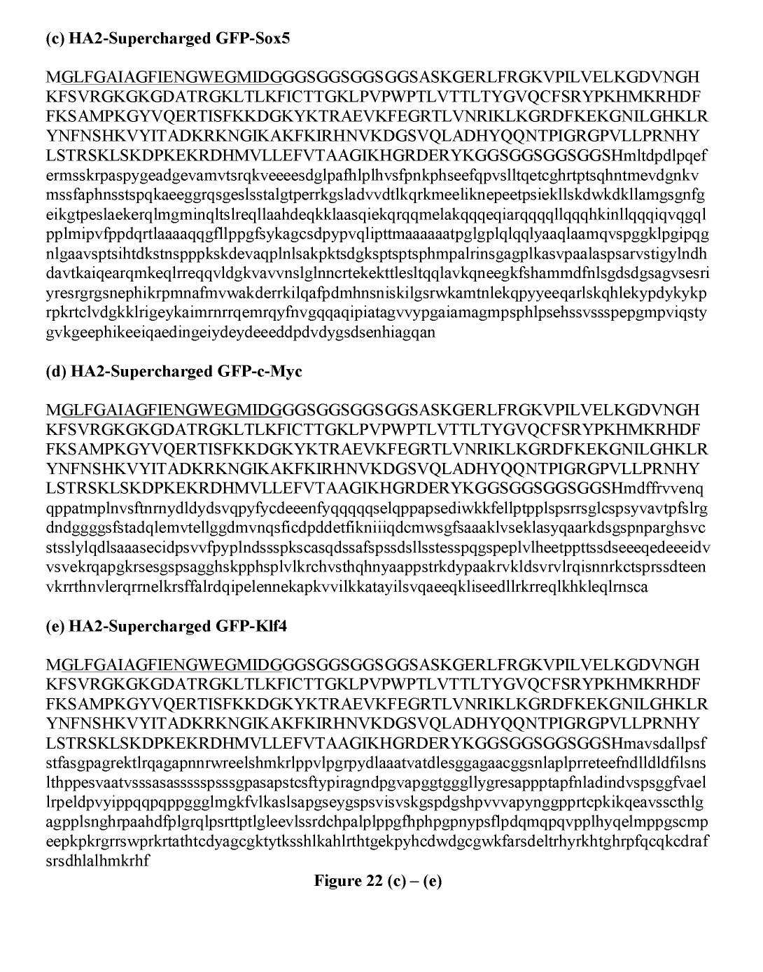

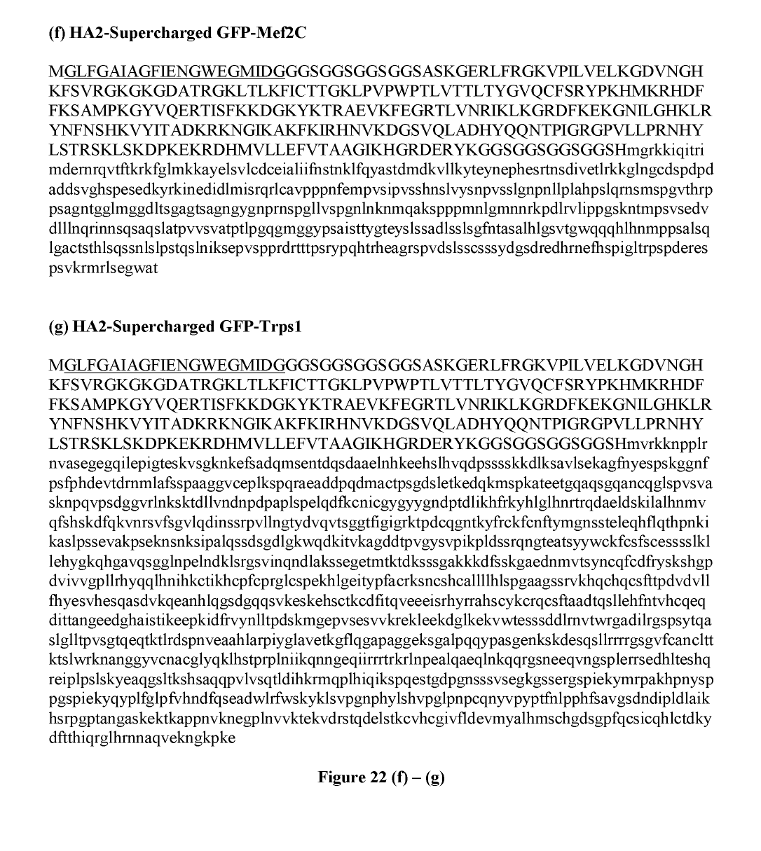

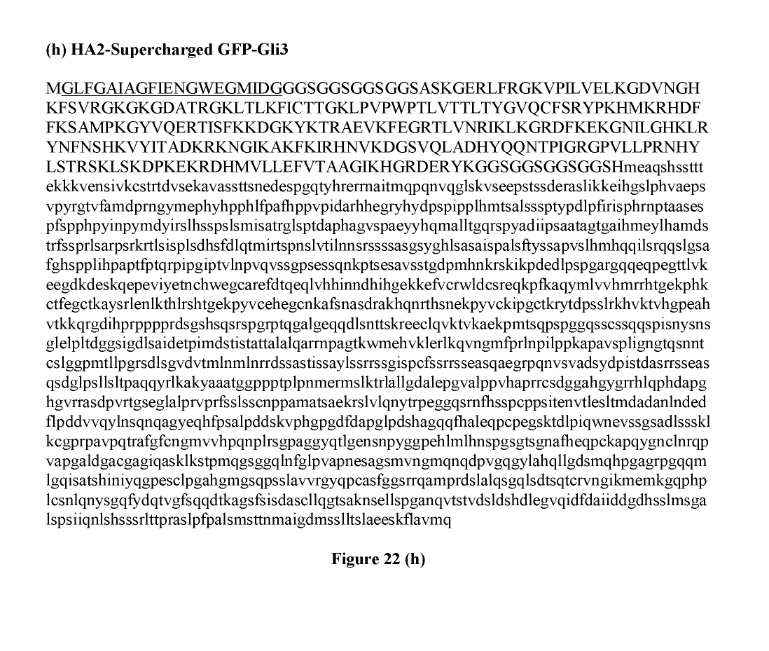

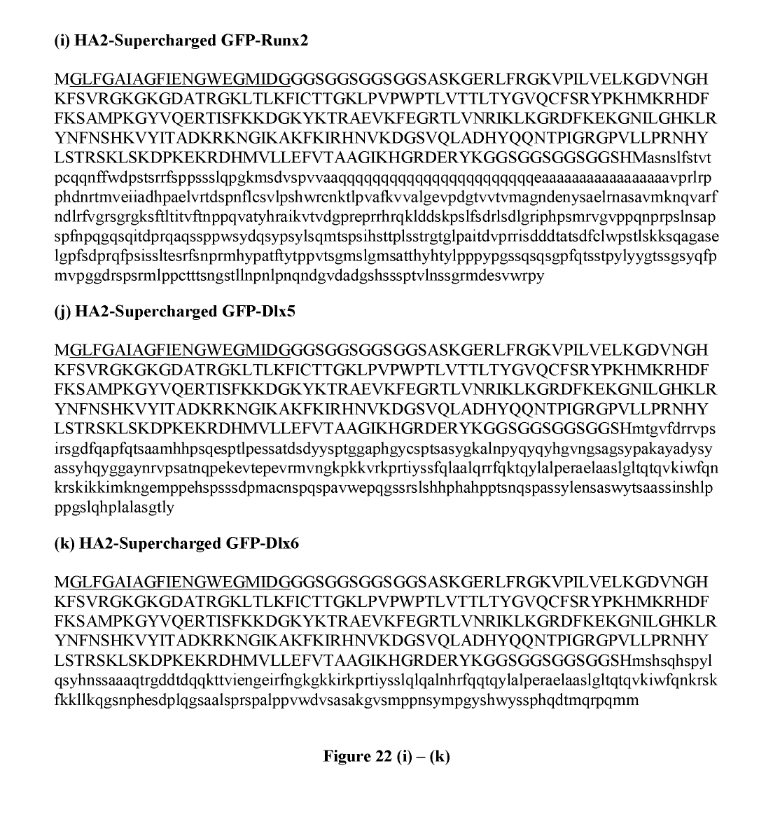

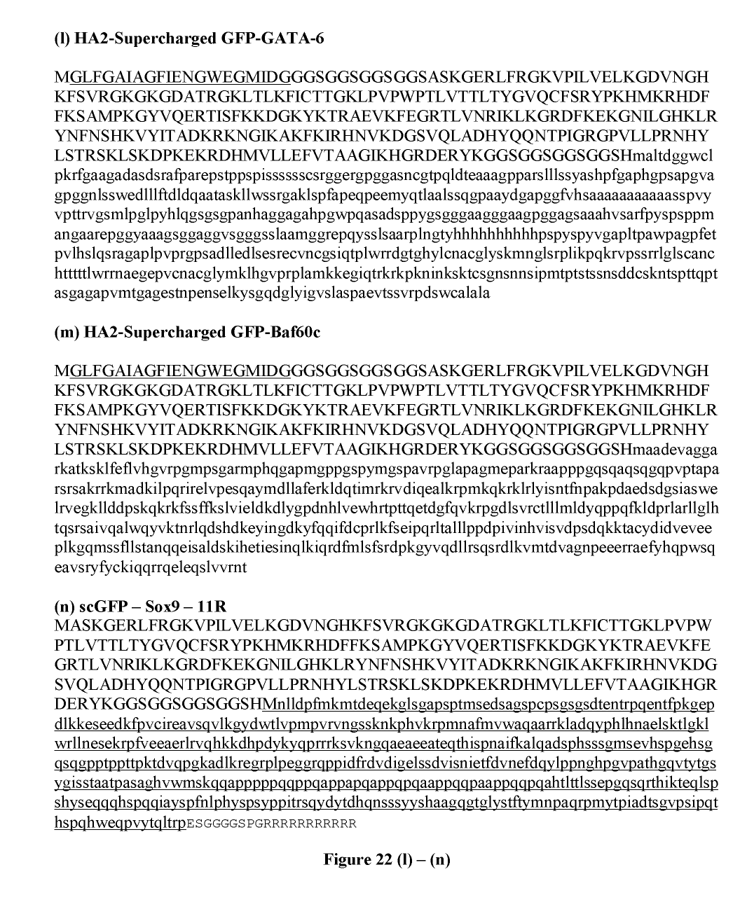

In certain embodiments, a transducible material includes, for example, Oct4-11R (SEQ ID NO: 12), Sox2-11R (SEQ ID NO: 13), Klf4-11R (SEQ ID NO: 14), Lin28-11R (SEQ ID NO: 16), Nanog-11R (SEQ ID NO: 17), cMyc-11R (SEQ ID NO: 15), Ngn3-11R (SEQ ID NO: 19), PDX1-11R (SEQ ID NO: 20), MafA-11R (SEQ ID NO: 21), NeuroD-11R (SEQ ID NO: 18), Foxp3-11R (SEQ ID NO: 22), Foxo1-11R (e.g. FIG. 18c), Foxo3-11R (e.g. FIG. 18d), Sox9-11R, Sox6-11R, Sox5-11R, c-Myc-11R, Klf4-11R, Mef2C-11R, Trps1-11R, Gli3-11R, Runx2-11R, Dlx5-11R, Dlx6-11R, GATA-6-11R and Baf60c-11R, wherein 11R (SEQ ID NO: 37) stands for a polyarginine sequence of 11 arginine residues linking to a linker through which the polyarginie sequence is covalently linked to the effector domain. In certain embodiments, the “11R” is covalently linked to the C terminal of the effector domain. In certain embodiments, a transducible material includes, for example, His6-Oct4-11R (SEQ ID NO: 23), His6Sox2-11R (SEQ ID NO: 24), His6-Klf4-11R (SEQ ID NO: 25), His6-Lin28-11R (SEQ ID NO: 27), His6-Nanog-11R (SEQ ID NO: 28), His6-cMyc-11R (SEQ ID NO: 26), His 6-Ngn3-11R (SEQ ID NO: 30), His6-PDX1-11R (SEQ ID NO: 31), His6-MafA-11R (SEQ ID NO: 32), His6-NeuroD-11R (SEQ ID NO: 29), His6-Foxp3-11R (SEQ ID NO: 33), His6-Foxo1-11R (e.g. FIG. 18e), His6-Foxo3-11R (e.g. FIG. 180, His6Sox9-11R, His6-Sox6-11R, His6-Sox5-11R, His6-c-Myc-11R, His6-Klf4-11R, His6-Mef2C-11R, His6-Trps1-11R, His6-Gli3-11R, His6-Runx2-11R, His6-Dlx5-11R, His6-Dlx6-11R, His6-GATA-6-11R, His6-Baf60c-11R, HA2-Supercharged GFP-Sox9, HA2-Supercharged GFP-Sox6, HA2-Supercharged GFP-Sox5, HA2-Supercharged GFP-c-Myc, HA2-Supercharged GFP-Klf4, HA2-Supercharged GFP-Mef2C, HA2-Supercharged GFP-Trps1, HA2-Supercharged GFP-Gli3, HA2-Supercharged GFP-Runx2, HA2-Supercharged GFP-Dlx5, HA2-Supercharged GFP-Dlx6, HA2-Supercharged GFP-GATA-6, HA2-Supercharged GFP-Baf60c, scGFP-Sox9-11R, scGFP-Sox6-11R, scGFP-Sox5-11R, scGFP-c-Myc-11R, scGFP-Klf4-11R, scGFP-Mef2C-11R, scGFP-Trps1-11R, scGFP-Gli3-11R, scGFP-Runx2-11R, scGFP-Dlx5-11R, scGFP-Dlx6-11R, scGFP-GATA-6-11R, and scGFP-Baf60c-11R. In certain embodiments, the “His6” is covalently linked to the N terminal of the effector domain. In certain embodiments, the N-terminal of the transcription factor is linked to scGFP via a first linker (e.g., GGSGGSGGSGGSH), and the C-terminal of the transcription factor is covalently linked to 11R with or without a second linker. A schematic presentation of a scGFP fused transcription material is shown in FIG. 1 (c), and amino acid sequence of an exemplary scGFP fused transcription material (i.e. scGFP-Sox9-11R) is provided in FIG. 22 (n), in which the sequence of the transcription factor Sox9 is underlined. By replacing the underlined Sox9 sequence with another transcription factor sequence, one can obtain the sequences of the scGFP fused transcription materials for the transcription factors, e.g. those listed in Table 1. Exemplary sequences of transducible materials are shown in Table 3.

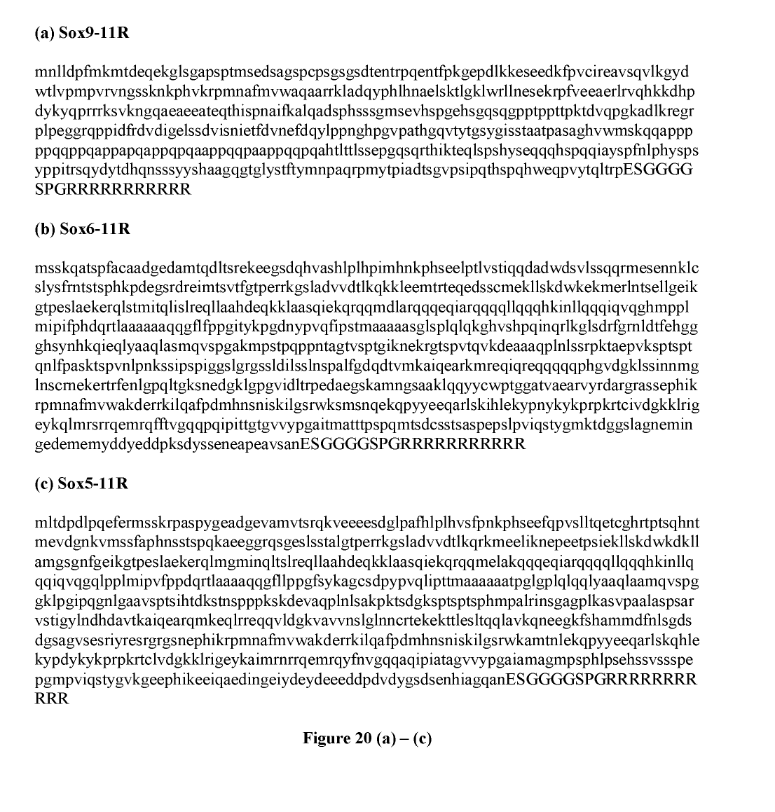

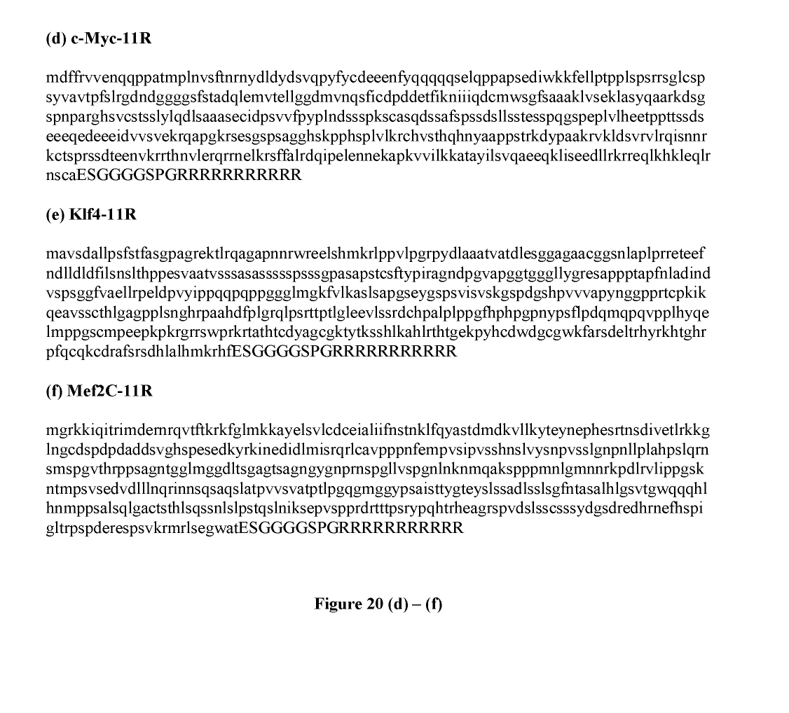

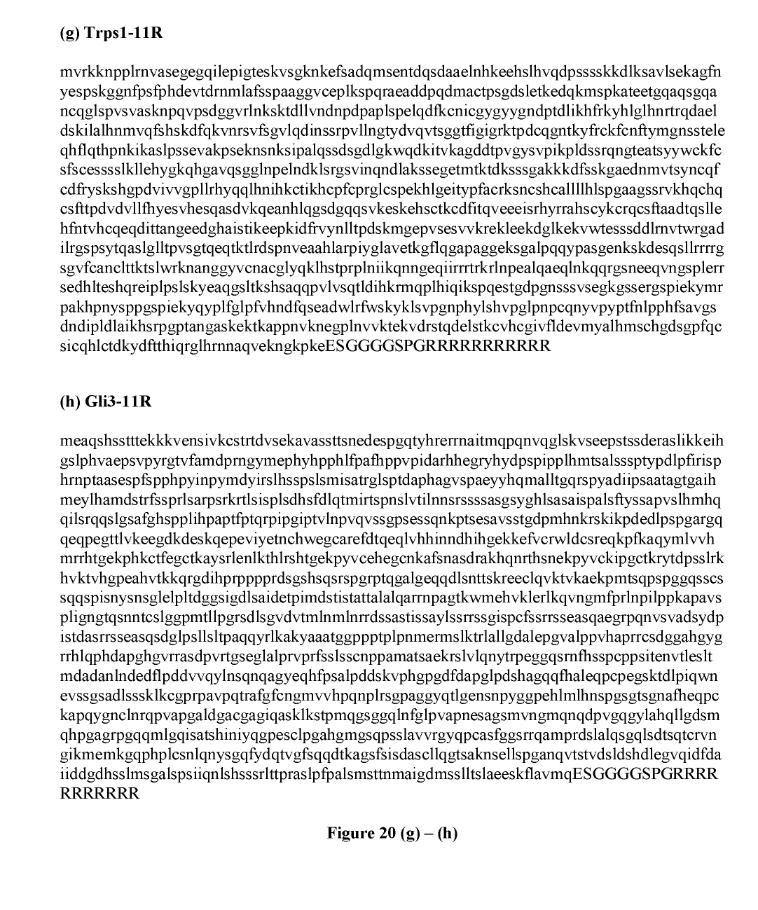

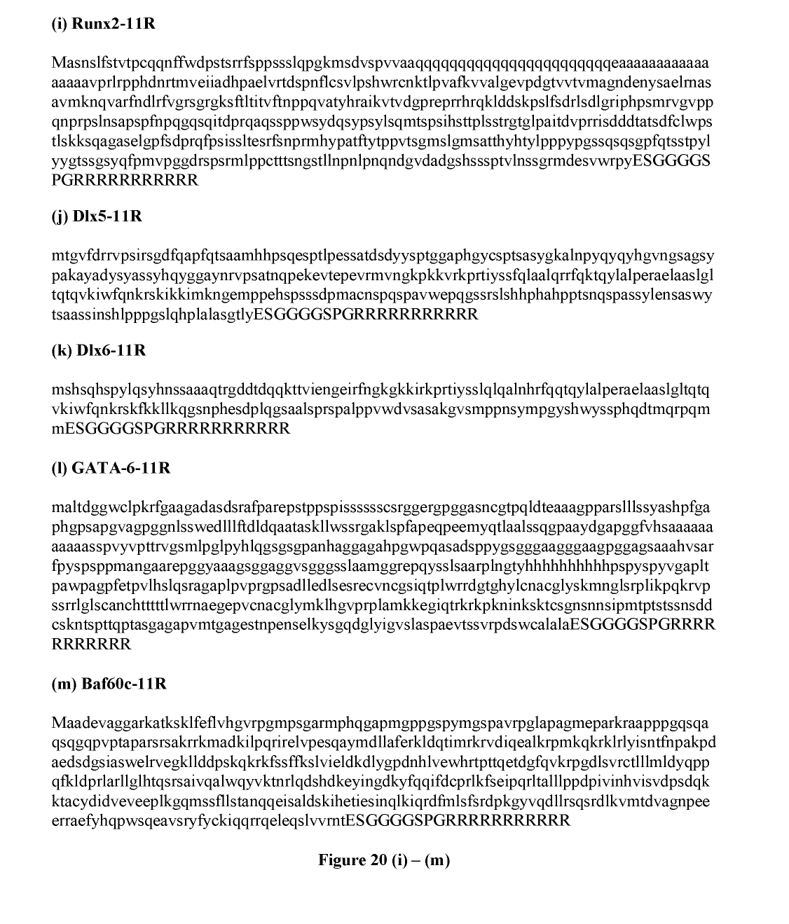

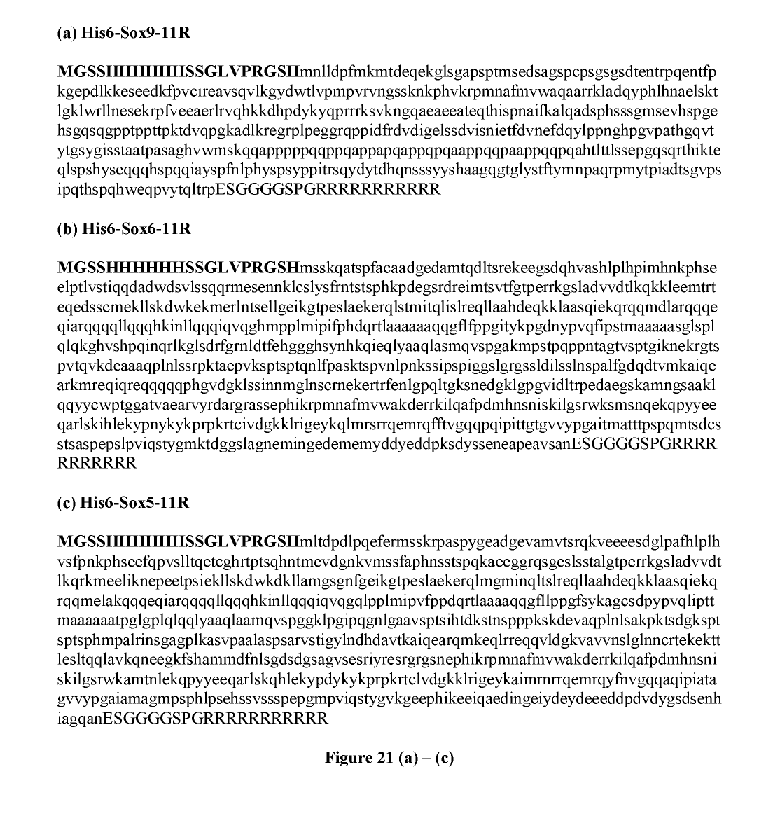

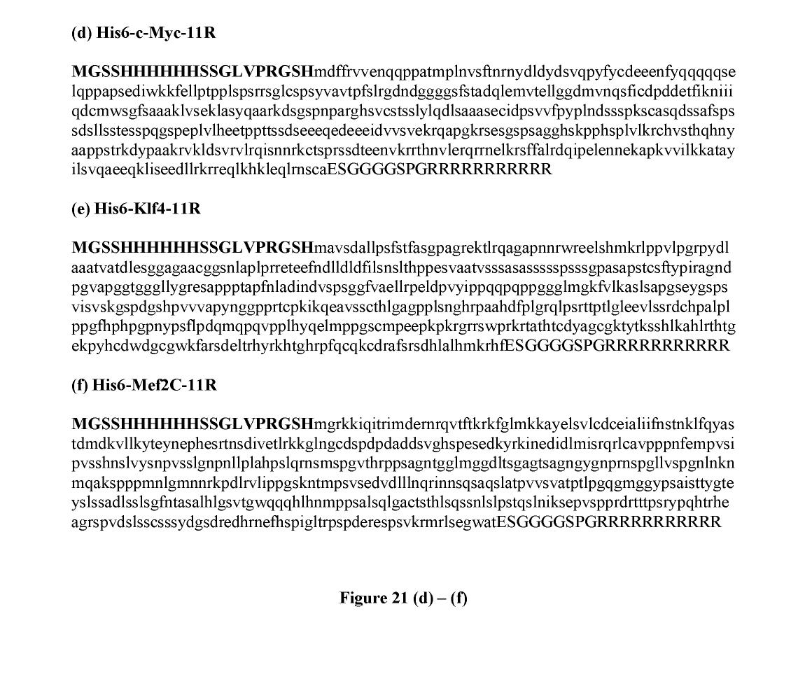

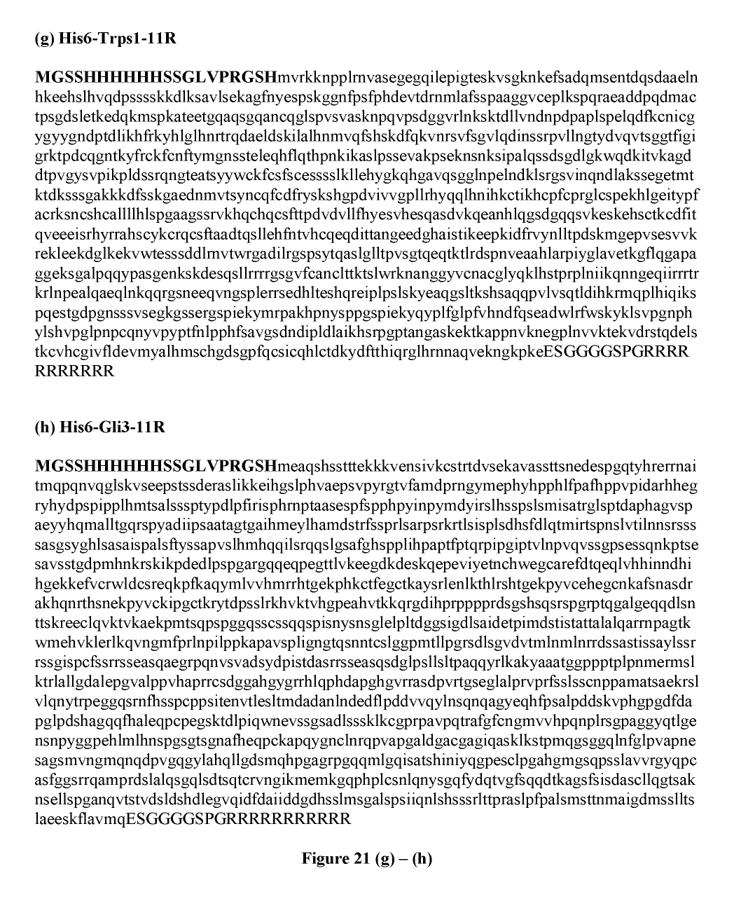

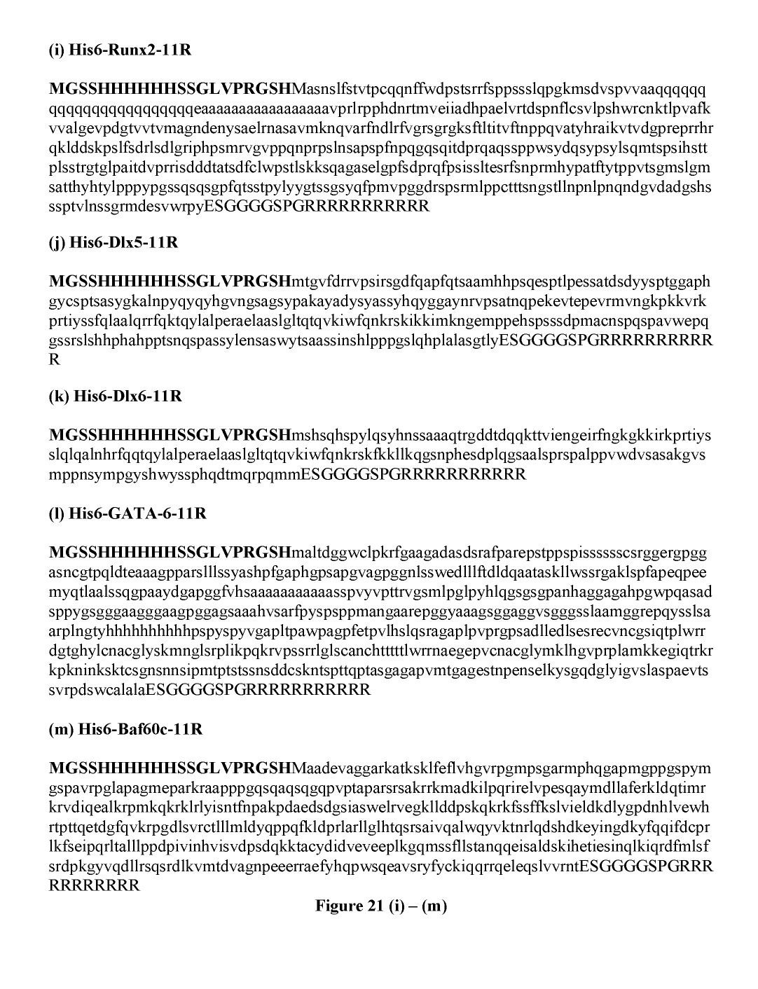

TABLE 3
Exemplary sequences of transducible materials

| | | His6-Effector | HA2-Supercharged GFP- |
|---|---|---|---|
| Effector | Effector domain-11R | domain-11R | Effector domain |
| Domain | (Exemplary sequence) | (Exemplary sequence) | (Exemplary sequence) |
| Sox9 | Sox9-11R | His6-Sox9-11R | HA2-Supercharged GFP- |
| | (FIG. 20 (a)) | (FIG. 21 (a)) | Sox9 |
| | | | (FIG. 22 (a)) |
| | | | scGFP-Sox9-11R (FIG. |
| | | | 22 (n), SEQ ID NO: 68) |
| Sox6 | Sox6-11R | His6-Sox6-11R | HA2-Supercharged GFP- |
| | (FIG. 20 (b)) | (FIG. 21 (b)) | Sox6 |
| | | | (FIG. 22 (b)) |
| Sox5 | Sox5-11R | His6-Sox5-11R | HA2-Supercharged GFP- |
| | (FIG. 20 (c)) | (FIG. 21 (c)) | Sox5 |
| | | | (FIG. 22 (c)) |
| c-Myc | c-Myc-11R | His6-c-Myc-11R | HA2-Supercharged GFP- |
| | (FIG. 20 (d)) | (FIG. 21 (d)) | c-Myc |
| | | | (FIG. 22 (d)) |
| Klf4 | Klf4-11R | His6-Klf4-11R | HA2-Supercharged GFP- |
| | (FIG. 20 (e)) | (FIG. 21 (e)) | Klf4 |
| | | | (FIG. 22 (e)) |
| Mef2C | Mef2C-11R | His6-Mef2C-11R | HA2-Supercharged GFP- |
| | (FIG. 20 (f)) | (FIG. 21 (f)) | Mef2C |
| | | | (FIG. 22 (f)) |
| Trps1 | Trps1-11R | His6-Trps1-11R | HA2-Supercharged GFP- |
| | (FIG. 20 (g)) | (FIG. 21 (g)) | Trps1 |
| | | | (FIG. 22 (g)) |
| Gli3 | Gli3-11R | His6-Gli3-11R | HA2-Supercharged GFP- |
| | (FIG. 20 (h)) | (FIG. 21 (h)) | Gli3 |
| | | | (FIG. 22 (h)) |
| Runx2 | Runx2-11R | His6-Runx2-11R | HA2-Supercharged GFP- |
| | (FIG. 20 (i)) | (FIG. 21 (i)) | Runx2 |
| | | | (FIG. 22 (i)) |
| Dlx5 | Dlx5-11R | His6-Dlx5-11R | HA2-Supercharged GFP- |
| | (FIG. 20 (j)) | (FIG. 21 (j)) | Dlx5 |
| | | | (FIG. 22 (j)) |
| Dlx6 | Dlx6-11R | His6-Dlx6-11R | HA2-Supercharged GFP- |
| | (FIG. 20 (k)) | (FIG. 21 (k)) | Dlx6 |
| | | | (FIG. 22 (k)) |
| GATA-6 | GATA-6-11R | His6-GATA-6-11R | HA2-Supercharged GFP- |
| | (FIG. 20 (l)) | (FIG. 21 (l)) | GATA-6 |
| | | | (FIG. 22 (l)) |
| Baf60c | Baf60c-11R | His6-Baf60c-11R | HA2-Supercharged GFP- |
| | (FIG. 20 (m)) | (FIG. 21 (m)) | Baf60c |
| | | | (FIG. 22 (m)) |

In certain embodiments, a transducible material can be combined with one or more adjuvants such as small molecule epigenetic agents. Suitable epigenetic agents include, without limitation, histone deacetylase inhibitor and DNA methylation inhibitor. Examples of suitable adjuvants include, without limitation, trichostatin A, which is a histone deacetylase inhibitor and DNA methylation inhibitor, valproic acid, which is a histone deacetylase inhibitor and DNA methylation inhibitor, aza-2′-deoxycytidine, which is a DNA methylation inhibitor, and suberoylanilide hydroxamic acid, which is a histone deacetylase inhibitor.

Another aspect of the present disclosure relates to a composition comprising a biological sample and at least one transducible material, wherein the transducible material has transduced into the biological sample. For example, the composition includes a transducible material comprising Foxp3, Foxo1, or Foxo3, or any combination thereof (e.g. the transducible material is Foxp3, Foxp3-11R, His6-Foxp3-11R, Foxo1, Foxo1-11R, His6-Foxo1-11R, Foxo3, Foxo3-11R, or His6-Foxo3-11R) and a T cell wherein the transducible material has transduced into the T cell; a composition includes a piPS cell and one or more transducible materials comprising a polypeptide selected from the group consisting of Oct4, Klf4, Sox2 and cMyc, and any combination thereof (e.g. the transducible material is Oct4, Klf4, Sox2, cMyc, Oct4-11R, Klf4-11R, Sox2-11R, cMyc-11R, His6-Oct4-11R, His6-Klf4-11R, His6-Sox2-11R or His6-cMyc-11R); a composition including a liver or pancreatic exocrine cell and one or more transducible materials comprising a polypeptide selected from the group consisting of Ngn3, PDX1, MafA, NeuroD, and any combination thereof (e.g. the transducible material is Ngn3, PDX1, MafA, NeuroD, Ngn3-11R, PDX1-11R, MafA-11R, NeuroD-11R, His6-Ngn3-11R, His 6-PDX1-11R or His6-MafA-11R, His6-NeuroD-11R) wherein the transducible materials have transduced into the liver or pancreatic exocrine cell; and a composition including a starting or substrate cell (e.g. embryonic stem cells (ESCs), induced pluripotent stem cells (iPSCs), mesenchymal stem cells (MSCs), fibroblasts, hematopoietic stem cells, endothelian stem cells, adipocytes, chondrocytes, osteoblasts, osteoclasts and endothelial cells) and one or more transducible materials comprising a polypeptide selected from the group consisting of Sox9, Sox6, Sox5, c-Myc, Klf4, Mef2C, Trps1, Gli3, Runx2, Dlx5, Dlx6, GATA-6 and Baf60c, and any combination thereof (e.g. the transducible material is Sox9-11R, Sox6-11R, Sox5-11R, c-Myc-11R, Klf4-11R, Mef2C-11R, Trps1-11R, Gli3-11R, Runx2-11R, Dlx5-11R, Dlx6-11R, GATA-6-11R, Baf60c-11R, His6-Sox9-11R, His6-Sox6-11R, His6-Sox5-11R, His6-c-Myc-11R, His6-Klf4-11R, His6-Mef2C-11R, His6-Trps1-11R, His6-Gli3-11R, His6-Runx2-11R, His6-Dlx5-11R, His6-Dlx6-11R, His6-GATA-6-11R, His6-Baf60c-11R, HA2-Supercharged GFP-Sox9, HA2-Supercharged GFP-Sox6, HA2-Supercharged GFP-Sox5, HA2-Supercharged GFP-c-Myc, HA2-Supercharged GFP-Klf4, HA2-Supercharged GFP-Mef2C, HA2-Supercharged GFP-Trps1, HA2-Supercharged GFP-Gli3, HA2-Supercharged GFP-Runx2, HA2-Supercharged GFP-Dlx5, HA2-Supercharged GFP-Dlx6, HA2-Supercharged GFP-GATA-6, HA2-Supercharged GFP-Baf60c, scGFP-Sox9-11R, scGFP-Sox6-11R, scGFP-Sox5-11R, scGFP-c-Myc-11R, scGFP-Klf4-11R, scGFP-Mef2C-11R, scGFP-Trps1-11R, scGFP-Gli3-11R, scGFP-Runx2-11R, scGFP-Dlx5-11R, scGFP-Dlx6-11R, scGFP-GATA-6-11R, scGFP-Baf60c-11R or any combination thereof) wherein the transducible materials have transduced into the starting cell.

Another aspect of the present disclosure relates to a method of reprogramming a biological sample by exposing the biological sample to a composition comprising a transducible material of the present disclosure. In certain embodiments, the method preferably reprograms a specific type of biological sample (e.g. cancer or tumor cells) or biological samples in or around a specific microenvironment within a biological organism (e.g. microenvironment around cancer or tumor) than other biological samples by exposing biological samples to a composition comprising a selective transducible material.

In one embodiment, a biological sample includes a cell, a cluster of cells, a tissue, an organ, a biological body from a biological organism. The biological sample can be normal, healthy sample or abnormal, diseased sample (e.g., cancer or tumor).

A biological organism includes, for example, a microorganism (e.g., bacteria), a fungus, a plant and an animal (e.g., a human).

An organ from an animal biological organism (e.g., human) includes, for example, a circulatory organ (e.g., heart, blood and blood vessels), a digestive organ (e.g., salivary glands, esophagus, stomach, liver, gallbladder, pancreas, intestines, rectum and anus), an endocrine organ (e.g., endocrine glands such as the hypothalamus, pituitary or pituitary gland, pineal body or pineal gland, thyroid, parathyroids and adrenals, i.e., adrenal glands), an integumentary organ (e.g., skin, hair and nails), a lymphatic organ (e.g., lymph nodes and vessels, tonsils, adenoids, thymus and spleen), a muscular organ (e.g., muscles), a nervous organ (e.g., brain, spinal cord, peripheral nerves and nerves), a reproductive organ (e.g., ovaries, fallopian tubes, uterus, vagina, mammary glands, testes, vas deferens, seminal vesicles, prostate and penis), a respiratory organ (e.g., the pharynx, larynx, trachea, bronchi, lungs and diaphragm), a skeletal organ (e.g., bones, cartilage, ligaments and tendons), a urinary system (e.g., kidneys, ureters, bladder and urethra). An organ can be normal or healthy, and alternatively, abnormal or unhealthy (e.g., cancerous).

An organ from a plant biological organism includes, for example, root, stem, leaf, flower, seed and fruit.

A tissue from a biological sample (e.g. an animal) includes a connective tissue, a muscle tissue, a nervous tissue, and an epithelial tissue. A tissue can be normal or healthy, and alternatively, abnormal or unhealthy (e.g., cancerous). A tissue from a biological sample (e.g. a plant) includes an epidermis, a vascular tissue and a ground tissue.

A cell can be prokaryotic or eukaryotic. A prokaryotic cell includes, for example, bacteria. A eukaryotic cell includes, for example, a fungus, a plant cell, and an animal cell. The types of an animal cell (e.g., a mammalian cell or a human cell) includes, for example, a cell from circulatory/immune system or organ (e.g., a B cell, a T cell (cytotoxic T cell, natural killer T cell, regulatory T cell, T helper cell), a natural killer cell, a granulocyte (e.g., basophil granulocyte, an eosinophil granulocyte, a neutrophil granulocyte and a hypersegmented neutrophil), a monocyte or macrophage, a red blood cell (e.g., reticulocyte), a mast cell, a thrombocyte or megakaryocyte, and a dendritic cell); a cell from an endocrine system or organ (e.g., a thyroid cell (e.g., thyroid epithelial cell, parafollicular cell), a parathyroid cell (e.g., parathyroid chief cell, oxyphil cell), an adrenal cell (e.g., chromaffin cell), and a pineal cell (e.g., pinealocyte)); a cell from a nervous system or organ (e.g., a glioblast (e.g., astrocyte and oligodendrocyte), a microglia, a magnocellular neurosecretory cell, a stellate cell, a boettcher cell, and a pituitary cell (e.g., gonadotrope, corticotrope, thyrotrope, somatotrope, and lactotroph)); a cell from a respiratory system or organ (e.g., a pneumocyte (a type I pneumocyte and a type II pneumocyte), a clara cell, a goblet cell, an alveolar macrophage); a cell from circular system or organ (e.g., myocardiocyte and pericyte); a cell from digestive system or organ (e.g., a gastric chief cell, a parietal cell, a goblet cell, a paneth cell, a G cell, a D cell, an ECL cell, an I cell, a K cell, an S cell, an enteroendocrine cell, an enterochromaffin cell, an APUD cell, a liver cell (e.g., a hepatocyte and Kupffer cell)); a cell from integumentary system or organ (e.g., a bone cell (e.g., an osteoblast, an osteocyte, and an osteoclast), a teeth cell (e.g., a cementoblast, and an ameloblast), a cartilage cell (e.g., a chondroblast and a chondrocyte), a skin/hair cell (e.g., a trichocyte, a keratinocyte, and a melanocyte (Nevus cell)), a muscle cell (e.g., myocyte), an adipocyte, a fibroblast, and a tendon cell), a cell from urinary system or organ (e.g., a podocyte, a juxtaglomerular cell, an intraglomerular mesangial cell, an extraglomerular mesangial cell, a kidney proximal tubule brush border cell, and a macula densa cell), and a cell from reproductive system or organ (e.g., a spermatozoon, a sertoli cell, a leydig cell, an ovum, an oocyte). A cell can be normal, healthy cell; or a diseased or unhealthy cell (e.g., a cancer cell).

A cell further includes a mammalian stem cell which include an embryonic stem cell, a fetal stem cell, an induced pluripotent stem cell, and an adult stem cell. A stem cell is a cell that is capable of undergoing cycles of cell division while maintaining an undifferentiated state and differentiating into specialized cell types. A stem cell can be an omnipotent stem cell, a pluripotent stem cell, a multipotent stem cell, an oligopotent stem cell and an unipotent stem cell (See, Hans R. Schöler (2007). “The Potential of Stem Cells: An Inventory” in Nikolaus Knoepffler, Dagmar Schipanski, and Stefan Lorenz Sorgner. Humanbiotechnology as Social Challenge. Ashgate Publishing, Ltd. pp. 28), any of which may be induced from a somatic cell. A stem cell may also include a cancer stem cell.

In certain embodiments, the cell is embryonic stem cells (ESCs), induced pluripotent stem cells (iPSCs), MSCs, fibroblasts, hematopoietic stem cells, endothelian stem cells, adipocytes, chondrocytes, osteoblasts, osteoclasts or endothelial cells.

In another embodiment, “reprogramming a biological sample” used herein is exchangeable with or refers to modulating, altering, or changing the biological activities of the biological sample (e.g., cell) or modulating, altering, or changing the state or status of the biological sample from one to another. For example, by exposing a biological sample (e.g., a cell) to a transducible material, the biological activities of the cell (e.g., cell growth, cell division, cell metabolism, cell cycle, cell signaling, DNA replication, transcription, RNA splicing, protein synthesis, post-translation modification) are modulated or altered so as to lead to cell proliferation, differentiation (e.g., from progenitor cells to terminally differentiated cells), dedifferentiation (e.g., from terminally differentiated cells to pluripotent stem cells), transdifferentiation (e.g., from one type of terminally differentiated cells to another type of terminally differentiated cells), retrodifferentiation (e.g., from terminally differentiated cells to progenitor cells), transdertermination (e.g., from one type of progenitor cells to a type of terminally differentiated cells that are usually derived from another type of progenitor cells under natural conditions), apoptosis (e.g., cell death of cells or cancer cells), morphogenesis, and changes in the cell fate. For another example, the state of a biological sample can be altered or changed from abnormal or diseased state to normal or healthy state (e.g., from cancer cells to noncancer cells); from one cell type to another cell type (e.g., from undifferentiated stem cells to differentiated stem cells or specialized cells), from differentiated or specialized cells to undifferentiated cells or stem cells (e.g., an omnipotent stem cell, a pluripotent stem cell, a multipotent stem cell, an oligopotent stem cell and an unipotent stem cell)(e.g., from fibroblast cells to induced pluripotent stem cells (iPSCs)), from somatic cells to stem cells or induced stem cells, from one state of stem cells to another state of stem cells (e.g., from ominipotent stem cells to pluripotent stem cells), from one type of differentiated cells to another type of differentiated cells (e.g., T-cells to regulatory T cells, pancreatic exocrine cells to insulin-producing beta cells), or from starting cells (e.g. ESCs, iPSCs, MSCs, fibroblasts, hematopoietic stem cells, endothelian stem cells, adipocytes, chondrocytes, osteoblasts, osteoclasts or endothelial cells) to chondrogenic cells.

In another embodiment, a biological sample is exposed to a transducible material and reprogrammed. The biological sample can be exposed in vitro, in vivo or ex vivo. For example, the biological sample is exposed in vitro through contacting the sample with the transducible material in an environment outside of a living biological organism (e.g., in a cell culture system or a test tube). The biological sample is exposed in vivo through contacting the material with a biological organism containing the sample or introducing (e.g., through administration) the material into the organism. The transducible materials can be administered via any known administration route such as for example parenteral (e.g., subcutaneous, intraperitoneal, intravenous, including intravenous infusion, intramuscular, or intradermal injection) or non-parenteral (e.g., oral, intranasal, intraocular, sublingual, rectal, or topical) route. The biological sample is exposed ex vivo when the biological sample (e.g., a cell, a tissue or an organ) is taken outside the biological organism, contacted with the transducible material, and placed back to the same or different biological organisms. Examples of ex vivo exposures comprise removing a biological sample from the biological organism, exposing the biological sample to a transducible material, and transplanting the biological sample transduced with the transducible material back to the biological organism.

In certain embodiments, O G2-MEF cells are exposed to a composition comprising protein Oct4-11R, Sox2-11R, Klf4-11R and cMyc-11R and reprogrammed to induced pluripotent stem cells (iPSCs).

In certain embodiments, T cells are exposed to a composition comprising protein Foxp3-11R, His6-Foxp3-11R, Foxo1-11R, His6-Foxo1-11R, His6-Foxo3-11R, Foxo3-11R, and reprogrammed to regulatory T cells (Treg cells).

In certain embodiments liver and/or pancreatic exocrine cells are exposed to a composition comprising one or more proteins selected from the group consisting of Ngn3-11R, PDX1-11R, MafA-11R, NeuroD-11R, His6-Ngn3-11R, His6-PDX1-11R, His 6-MafA-11R, and His6-NeuroD-11R and reprogrammed into insulin producing cells (e.g. β cells). In certain embodiments, the composition further comprises one or more adjuvant such as Islet growth factor (e.g. betacellulin). In certain embodiments, the composition comprises His6-Ngn3-11R, His6-PDX1-11R, and His6-MafA-11R. In certain embodiments, the composition comprises His6-Ngn3-11R, His6-PDX1-11R, His 6-MafA-11R and betacellulin. Without intending to be bound to a particular mechanism, it is further contemplated that such reprogramming is through transdetermination and/or transdifferentiation.

In certain embodiments, MSCs, ESCs, iPSCs, fibroblasts, hematopoietic stem cells, endothelian stem cells, adipocytes, chondrocytes, osteoblasts, osteoclasts or endothelial cells are exposed to a composition comprising one or more proteins selected from the group consisting of Sox9-11R, Sox6-11R, Sox5-11R, c-Myc-11R, Klf4-11R, Mef2C-11R, Trps1-11R, Gli3-11R, Runx2-11R, Dlx5-11R, Dlx6-11R, GATA-6-11R, Baf60c-11R, His6-Sox9-11R, His6-Sox6-11R, His6-Sox5-11R, His6-c-Myc-11R, His6-Klf4-11R, His6-Mef2C-11R, His6-Trps1-11R, His6-Gli3-11R, His6-Runx2-11R, His6-Dlx5-11R, His6-Dlx6-11R, His6-GATA-6-11R, His6-Baf60c-11R, HA2-Supercharged GFP-Sox9, HA2-Supercharged GFP-Sox6, HA2-Supercharged GFP-Sox5, HA2-Supercharged GFP-c-Myc, HA2-Supercharged GFP-Klf4, HA2-Supercharged GFP-Mef2C, HA2-Supercharged GFP-Trps1, HA2-Supercharged GFP-Gli3, HA2-Supercharged GFP-Runx2, HA2-Supercharged GFP-Dlx5, HA2-Supercharged GFP-Dlx6, HA2-Supercharged GFP-GATA-6 and HA2-Supercharged GFP-Baf60c, scGFP-Sox9-11R, scGFP-Sox6-11R, scGFP-Sox5-11R, scGFP-c-Myc-11R, scGFP-Klf4-11R, scGFP-Mef2C-11R, scGFP-Trps1-11R, scGFP-Gli3-11R, scGFP-Runx2-11R, scGFP-Dlx5-11R, scGFP-Dlx6-11R, scGFP-GATA-6-11R, and scGFP-Baf60c-11R and reprogrammed into chondrogenic cells. In certain embodiments, the composition further comprises one or more adjuvant such as epigenetic agents (e.g. trichostatin A, valproic acid, aza-2′-deoxycytidine, suberoylanilide hydroxamic acid).

Another aspect of the present disclosure relates to a method of treating, preventing or reducing a disease or condition involving cartilage damage in a biological organism by administering a composition comprising a transducible material of the present disclosure into the organism. In certain embodiments, the composition is a pharmaceutical composition comprising a transducible material. In certain embodiments, the composition comprises a selective transducible material. The treatment, prevention or reduction of a disease or condition is associated with the change or reprogramming of a biological sample (e.g., a cell, a tissue or an organ) in the organism.

The present disclosure also provides use of a transducible material in manufacturing a medicament for treating preventing or reducing a disease or condition involving cartilage damage in a biological organism. In certain embodiments, the transducible material is a selective transducible material. The treatment, prevention or reduction of a disease or condition is associated with the change or reprogramming of a biological sample (e.g., a cell, a tissue or an organ) in the organism. The disease or condition involving cartilage damage include, without limitations, joint disorder, osteoarthritis, cartilage injury, traumatic rupture or detachment, achondroplasia, costochondritis, spinal disc herniation, relapsing polychonritis, tumor, chondroma, chondrosarcoma, and peomorphic adenoma.

In certain embodiments, the disease or condition treatable by the method or treatable by the medicament include, without limitations, tumor, cancer, metabolic diseases or conditions (e.g. type I and type II diabetes and obesity), inflammatory conditions, cardiac diseases, neurogenerative diseases (e.g. anemia, amyotrophic lateral sclerosis, spinal cord injury, burns, or arthritis), autoimmune diseases or conditions (e.g. acute disseminated encephalomyelitis (ADEM), Addison's disease, alopecia greata, ankylosing spondylitis, antiphospholipid antibody syndrome (APS), anemia (e.g. autoimmune hemolytic anemia and pernicious anaemia), arthritis, psoriatic arthritis, rheumatoid arthritis, diabetes mellitus type 1, autoimmune hepatitis, autoimmune inner ear disease, bullous pemphigoid, coeliac disease, Chagas disease, chronic obstructive pulmonary disease, Crohns disease, dermatomyositis, endometriosis, Goodpasture's syndrome, Graves' disease, Guillain-Barre syndrome (GBS), Hashimoto's disease, hidradenitis suppurativa, Kawasaki disease, IgA nephropathy, idiopathic thrombocytopenic purpura, interstitial cyctitis, lupus erythematosus, mixed connective tissue disease, morphea, multiple sclerosis (MS), myasthenia gravis, narcolepsy, neuromyotonia, pemphigus vulgaris, psoriasis, polymyositis, primary billiary cirrhosis, schizophrenia, scleroderma, Sjogren's syndrome, stiff person syndrome, temporal arteritis (“Giant cell arteritis”), ulcerative colitis, vasculitis, vitiligo, and Wegener's granulomatosis).

For example, it is contemplated that a transducible material, or a medicament manufactured from a transducible material can be administered to a biological organism having a tumor to activate the apoptosis of the tumor cells or make tumor cells more sensitive to chemotherapy, radiotherapy, or cancer drugs.

In certain embodiments, a transducible material, or a medicament manufactured from a transducible material can be administered to a biological organism to enhance or attenuate immune system and thus treat or prevent immune-related diseases or inflammatory diseases. For example, protein Foxp3-11R, His6-Foxp3-11R, Foxo1-11R, His6-Foxo1-11R, His6-Foxo3-11R, Foxo3-11R, is transduced to T cells and programs them to Treg cells, which suppress the overactive immune system and thus is a treatment for auto-immune diseases.

In certain embodiments, a transducible material, or a medicament manufactured from a transducible material can be administered to a biological organism to treat a condition involving cartilage damage such as joint disorder, osteoarthritis, cartilage injury, cartilage injury, traumatic rupture or detachment, achondroplasia, costochondritis, spinal disc herniation, relapsing polychonritis, tumor, chondroma, chondrosarcoma, or peomorphic adenoma.

For example, to treat a condition involving cartilage damage, a polypeptide or a composition comprising such polypeptide is transduced to a suitable cell (e.g. ESCs, iPSCs, MSCs, fibroblasts, hematopoietic stem cells, endothelian stem cells, adipocytes, chondrocytes, osteoblasts, osteoclasts or endothelial cells) and reprograms them to chondrogenic cells, which polypeptide is selected from the group consisting of Sox9-11R, Sox6-11R, Sox5-11R, c-Myc-11R, Klf4-11R, Mef2C-11R, Trps1-11R, Gli3-11R, Runx2-11R, Dlx5-11R, Dlx6-11R, GATA-6-11R, Baf60c-11R, His6-Sox9-11R, His6-Sox6-11R, His 6-Sox5-11R, His6-c-Myc-11R, His6-Klf4-11R, His6-Mef2C-11R, His6-Trps1-11R, His 6-Gli3-11R, His6-Runx2-11R, His6-Dlx5-11R, His6-Dlx6-11R, His6-GATA-6-11R, His 6-Baf60c-11R, HA2-Supercharged GFP-Sox9, HA2-Supercharged GFP-Sox6, HA2-Supercharged GFP-Sox5, HA2-Supercharged GFP-c-Myc, HA2-Supercharged GFP-Klf4, HA2-Supercharged GFP-Mef2C, HA2-Supercharged GFP-Trps1, HA2-Supercharged GFP-Gli3, HA2-Supercharged GFP-Runx2, HA2-Supercharged GFP-Dlx5, HA2-Supercharged GFP-Dlx6, HA2-Supercharged GFP-GATA-6, HA2-Supercharged GFP-Baf60c, scGFP-Sox9-11R, scGFP-Sox6-11R, scGFP-Sox5-11R, scGFP-c-Myc-11R, scGFP-Klf4-11R, scGFP-Mef2C-11R, scGFP-Trps1-11R, scGFP-Gli3-11R, scGFP-Runx2-11R, scGFP-Dlx5-11R, scGFP-Dlx6-11R, scGFP-GATA-6-11R, scGFP-Baf60c-11R, and any combination thereof. Without intending to be bound to a particular mechanism, it is further contemplated that such reprogramming is through transdetermination and/or transdifferentiation.

For another example, starting cells or substrate cells (e.g. embryonic stem cells (ESCs), induced pluripotent stem cells (iPSCs), MSCs, fibroblasts, hematopoietic stem cells, endothelian stem cells, adipocytes, chondrocytes, osteoblasts, osteoclasts and endothelial cells) are reprogrammed to chondrogenic cells through differentiation, transdifferentiation, trans-determination, or retro-differentiation.

In certain embodiments, one or more adjuvant such as epigenetic agents (e.g. trichostatin A, valproic acid, aza-2′-deoxycytidine, and/or suberoylanilide hydroxamic acid) is/are also administered to the biological organism.

Another aspect of the present disclosure relates to a method of reprogramming iPSCs, embryonic stem cells, or other types of stem or progenitor or differentiated cells to chondrogenic cells, which can be developed as cell-based therapies for diseases or conditions involving cartilage damage including joint disorder, osteoarthritis, cartilage injury, traumatic rupture or detachment, achondroplasia, costochondritis, spinal disc herniation, relapsing polychonritis, tumor, chondroma, chondrosarcoma, or peomorphic adenoma. The stem cells, progenitor cells or differentiated cells may be patient-specific or non-patient-specific, repaired to rid of molecular defects or not, before they are exposed to transducible materials for controlled differentiation or reprogramming. The reprogrammed cells may be enriched, purified, or manipulated before transplanted back to patients. In certain embodiments, the reprogrammed cell is chondrogenic cells or their derived cells.

Another aspect of the present disclosure relates to a method of reprogramming iPSCs, embryonic stem cells, or other types of stem or progenitor cells or differentiated cells to chondrogenic cells, which can be used as disease models for drug screening, mechanism study, toxicity assay, or other research and drug discovery and development tools. For example, the method comprises exposing an iPSC, an embryonic stem cell, a progenitor cell or a differentiated cell to a composition comprising a transducible material to reprogram the iPSC, embryonic stem cell, progenitor cell or differentiated cell to a transplantable chondrogenic cell; transplanting the transplantable chondrogenic cell into a biological sample or a biological organism; developing the biological sample or biological organism to become a disease model. For another example, the method comprises reprogramming patient-specific cells to chondrogenic cells using a transducible materials; and developing a disease model using patient-specific chondrogenic cells or chondrogenic cell-derived cells. For another example, the method of developing drug screening or toxicity models comprises reprogramming somatic cells, progenitor cells, or multipotent cells to chondrogenic cells using a transducible material and using chondrogenic cells and/or chondrogenic cell-derived cells to screen the effects and/or toxicities of different compounds.

Another aspect of the present disclosure relates to a method of developing cell-based therapies for various diseases or conditions involving cartilage damage comprising the step of reprogramming an iPSC, an embryonic stem cell, a progenitor cell or a differentiated cell to a transplantable chondrogenic cell using a transducible material; transplanting the transplantable chondrogenic cell into a biological sample or biological organism; assessing the therapeutic effect of the transplantable chondrogenic cell.

The transplantable cells can be transplanted to a subject in need, and once in the right place, such as in the injury sites, blood clots, or the subchondral bone marrow space, the transplanted chondrogenic cells or their derived cells can regenerate cartilage tissue, preferably hyaline cartilage tissue and repair cartilage damage.

For example, a patient with a condition involving cartilage damage is treated with protein drugs, small molecule drugs, or the combination, through injection into the injured site of his/her articular cartilage, joint or subchondral bone marrow space, generating new chondrogenic cells at the injured sites, blood clots, or the subchondral bone marrow space. The newly generated chondrogenic cells regenerate hyaline cartilage tissue and repair cartilage damage.

Another aspect of the present disclosure relates to a method of identifying a effector domain, wherein the method comprises the steps of covalently linking a test effector domain to a know transduction domain to form a test transducible molecule; exposing the test molecule to a biological sample, and measuring the reprogramming of the biological sample to a chondrogenic sample to indicate whether the test effector domain can exerts a change in the biological sample. It is also contemplated that another aspect of the present disclosure relates to a method of identifying a transducible domain, wherein the method comprises the steps of covalently linking a known effector domain to a test transduction domain to form a test transducible molecule; exposing the test molecule to a biological sample, and measuring the location of the test molecule in or the reprogramming effect of the biological sample to a chondrogenic sample to indicate whether the test transduction domain can transduce the effector domain into the biological sample.

### EXAMPLES

The following examples are provided to better illustrate the claimed invention and are not to be interpreted in any way as limiting the scope of the invention. All specific compositions, materials, and methods described below, in whole or in part, fall within the scope of the invention. These specific compositions, materials, and methods are not intended to limit the invention, but merely to illustrate specific embodiments falling within the scope of the invention. One skilled in the art may develop equivalent compositions, materials, and methods without the exercise of inventive capacity and without departing from the scope of the invention. It will be understood that many variations can be made in the procedures herein described while still remaining within the bounds of the invention. It is the intention of the inventors that such variations are included within the scope of the invention.

### Example 1

### Reprogramming Somatic Cells to Induced Pluripotent Stem Cells (iPSCs)

1.a. Preparation of transducible material Oct4-11R, Sox2-11R, Klf4-11R, and cMyc-11R.

A poly-arginine protein transduction domain was fused to the C-terminal of each reprogramming proteins Oct4, Sox2, Klf4 and cMyc through a linker SEQ ID NO. 55 to form a fused protein Oct4-11R, Sox2-11R, Klf4-11R and cMyc-11R respectively (FIG. 1A). These poly-arginine fused proteins were expressed in E. Coli in inclusion body form, which were then solubilized, refolded, and further purified to render transducible materials Oct4-11R, Sox2-11R, Klf4-11R and cMyc-11R. The protein identities were confirmed by mass spectrometry and Western blot analysis (FIG. 1B).

1.b. Cell permeability and stability of transducible material Oct4-11R, Sox2-11R, Klf4-11R, and cMyc-11R

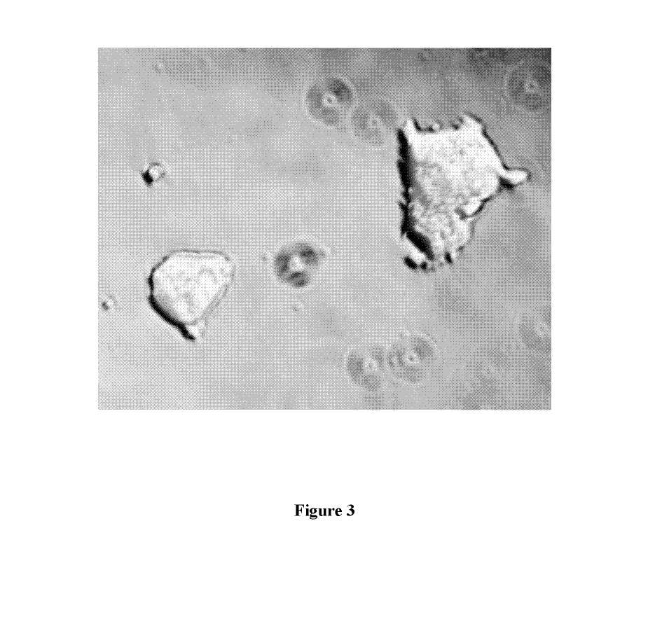

A transducible material (Oct4-11R, Sox2-11R, Klf4-11R, or cMyc-11R) was added to mouse embryonic fibroblast (MEF) cells at various concentrations for 6-72 hours. Cell morphology and protein presence were examined by immunocytochemistry. The transducible materials were found to enter cells at concentrations of 0.5-8 μg/ml within 6 hours, and translocated into nucleus (FIG. 2). In addition, the transduced proteins were fairly stable inside of cells for up to 48 hours (FIG. 3).

1.c. Reprogramming O G2/Oct4-GFP reporter MEF cells.

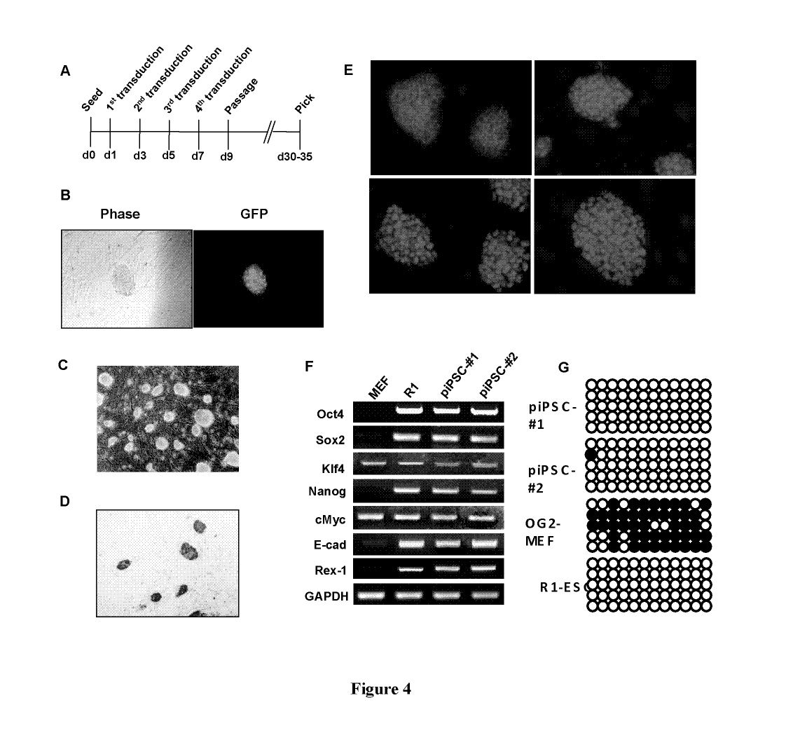

The protein transduction condition described in paragraph 0047 was used to reprogram O G2/Oct4-GFP reporter MEF cells. Cells were treated in 4 cycles. In each cycle the fibroblasts (initially seeded at the density of 5×10⁴ cells/well in a six-well plate) were first treated with transducible materials Oct4-11R, Sox2-11R, Klf4-11R and cMyc-11R at 8 μg/ml in the mESC growth media supplemented with or without 1 mM valproic acid (VPA, a inhibitor of the enzyme histone deacetylase 1 (HDAC1)) for overnight, followed by changing to the same media without the transducible material and VPA, and culturing for additional 36 hours before the next cycle of the treatment. After completing repeated protein transduction of a transducible material, the treated cells were transferred onto irradiated MEF feeder cells and kept in mESC growth media until colonies emerged around day 30-35 (FIG. 4A). 3 GFP+ colonies per 5×10⁴ cells were obtained when the cells were transduced with Oct4-11R, Sox2-11R, Klf4-11R, and cMyc-11R and treated with VPA, and 1 GFP+ colony per 5×10⁴ cells were obtained when the cells were transduced with Oct4-11R, Sox2-11R, or Klf4-11R respectively and treated with VPA. Those initial GFP+ colonies were subsequently passaged under conventional mESC growth conditions to yield piPS cells, and were further characterized.

The generated murine piPS cells have been stably expanded for over twenty passages, and were morphologically indistinguishable to classic mES cells, forming compact domed small colonies (FIGS. 4B and 4C). They expressed typical pluripotency markers examined by immunocytochemistry and staining, including ALP (FIG. 4D), Oct4, Nanog, Sox2, and SSEA1 (FIG. 4E). RT-PCR analysis confirmed endogenous gene expression of these pluripotency markers and additional pluripotency genes (FIG. 4F). A single cell survival assay also demonstrated that piPS cells clonally expanded efficiently as Oct4-positive colonies in feeder-free and N2/B27-chemically defined conditions. Furthermore, bisulphite genomic sequencing analyses of the Oct4 promoter revealed that it was demethylated in piPS cells similarly to the mES cells, while the MEFs' Oct4 promoter was hypermethylated (FIG. 4G). This result further suggests a reactivation of the pluripotency transcription program in these piPS cells.

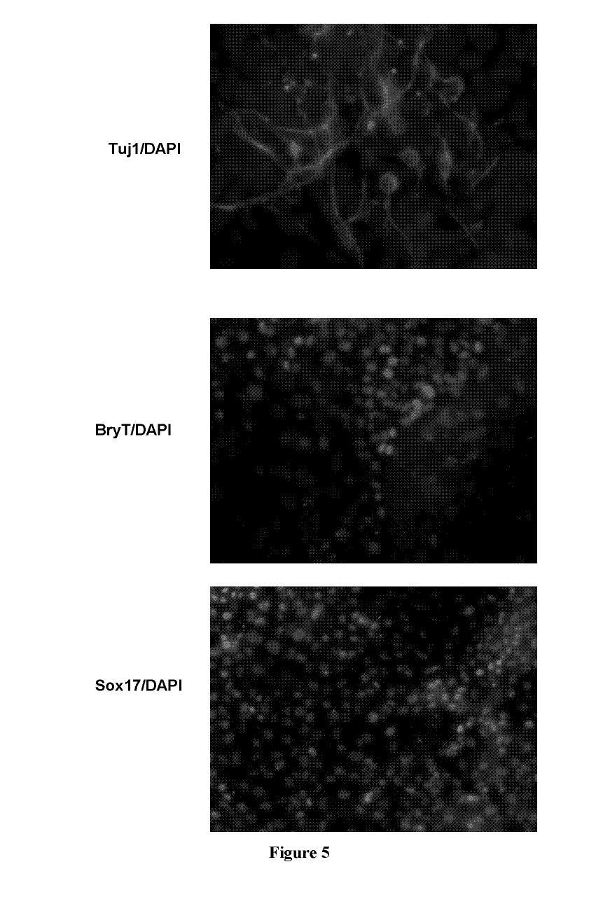

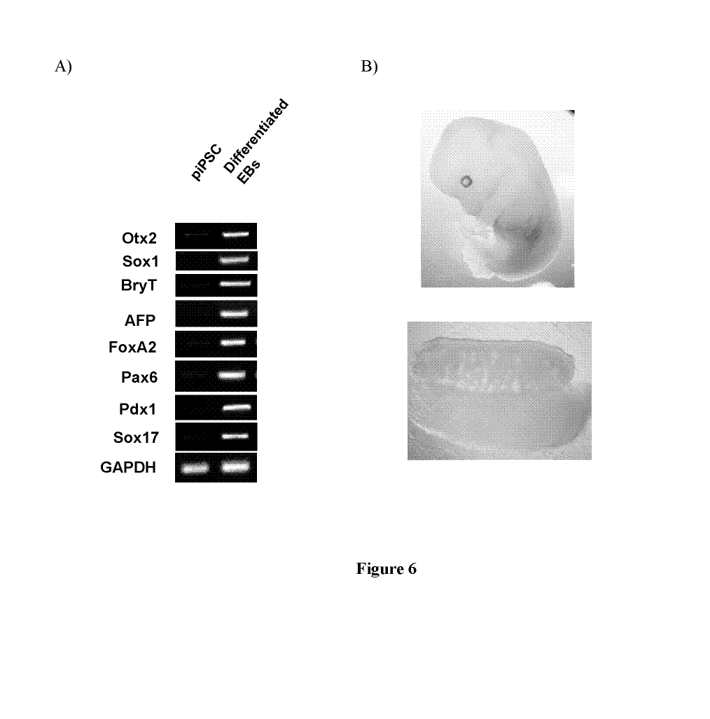

To examine the developmental potential of piPS cells, standard in vitro differentiation using embryoid bodies (EB) or monolayer chemically defined step-wise differentiation, as well as in vivo chimerism assays were performed. piPS cells efficiently formed EB in suspension, and differentiated into cells in the three primary germ layers, including primitive endoderm (AFP, Sox17), foregut endoderm (FoxA2), pancreatic cells endoderm (PDX1, Pax6), mesoderm (Brachyury), and neural (Sox1) and neuronal cells (βIII-tubulin)-ectoderm (FIGS. 5 and 6 A). These piPS cells efficiently incorporated into the inner cell mass of a blastocyst following aggregation with an 8-cell embryo, and led to chimerism with germline contribution (FIG. 6B) in vivo after the aggregated embryos were transplanted into mice, as suggested by observation of Oct4-GFP+ cells in the gonad tissue in 2 out of 7 embryos (FIG. 6B bottom). These in vitro and in vivo characterizations collectively confirm that the purified transduction material Oct4-11R, Sox2-11R, Klf4-11R, and cMyc-11R are able to reprogram MEFs to piPS cells, which are morphologically and functionally similar to conventional mES cells.

### Example 2

Reprogramming of liver and pancreatic exocrine cells to insulin-producing beta cells by transducible materials His6-Ngn3-11R, His6-PDX1-11R and His 6-MafA-11R in mouse.

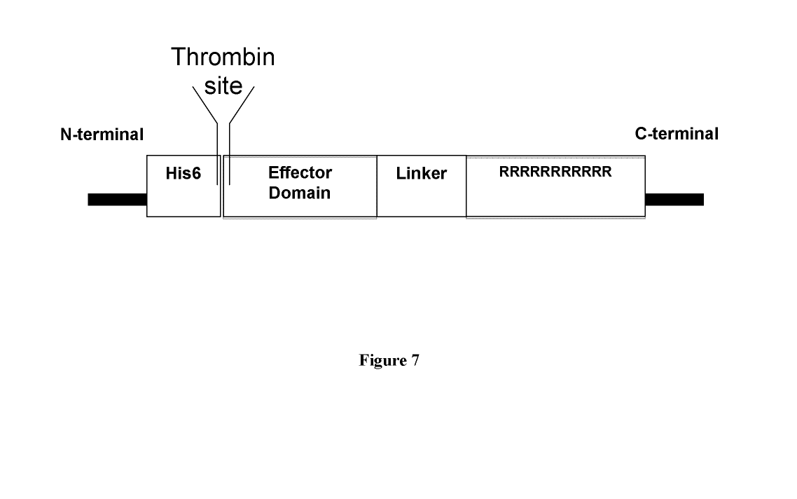

A poly-arginine protein transduction domain was fused respectively to the C-terminal of each reprogramming protein (Ngn3, PDX1 and MafA) through a linker (SEQ ID NO: 55) to form His6-Ngn3-11R, His6-PDX1-11R and His6-MafA-11R respectively (FIG. 7). His6 (SEQ ID NO: 59) was included to facilitate protein purification. These poly-arginine fused proteins were expressed in E. Coli in inclusion body form, which were then solubilized, refolded, and further purified to prepare transducible materials His 6-Ngn3-11R, His6-PDX1-11R and His6-MafA-11R. The protein identities were confirmed by mass spectrometry and Western blot analysis.

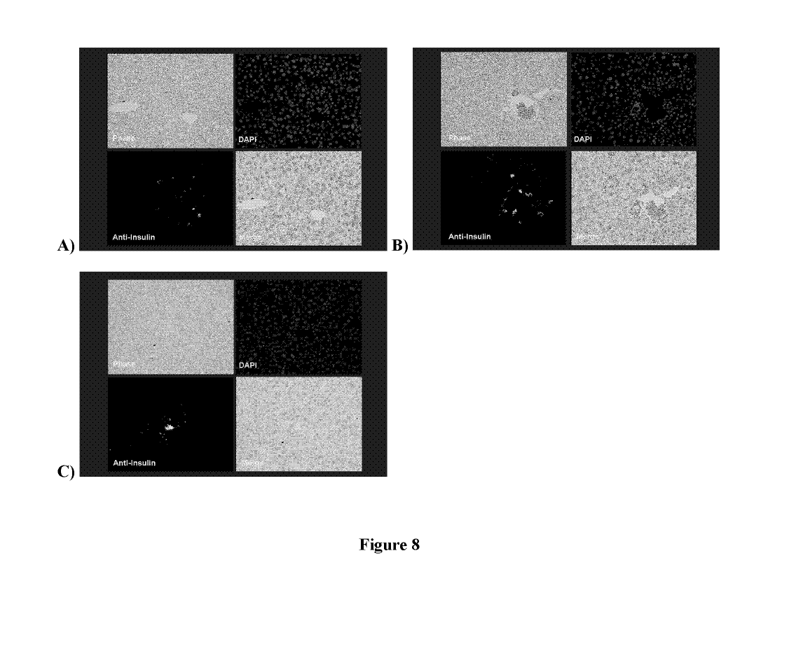

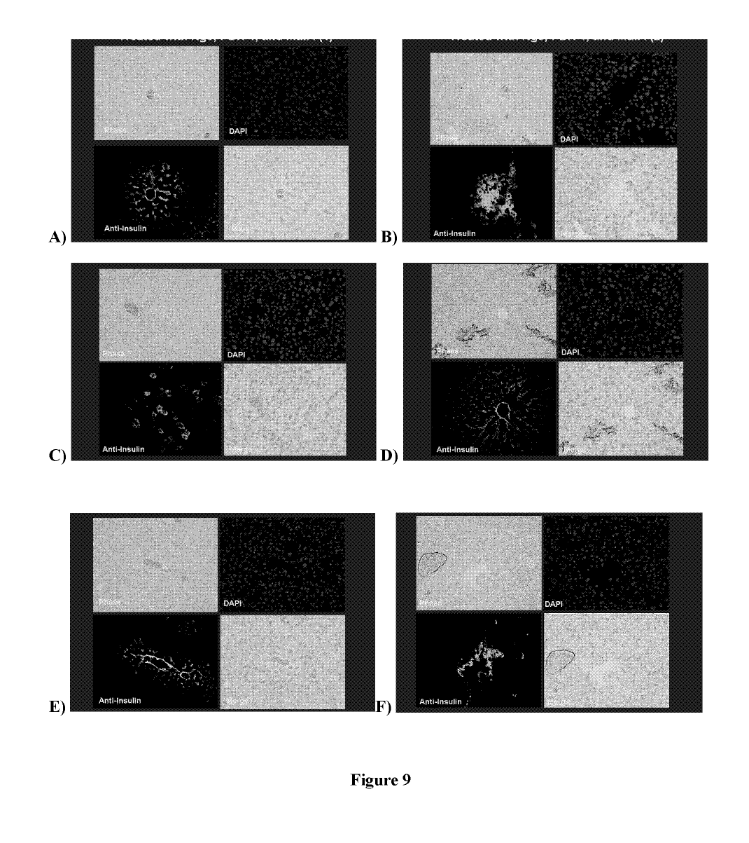

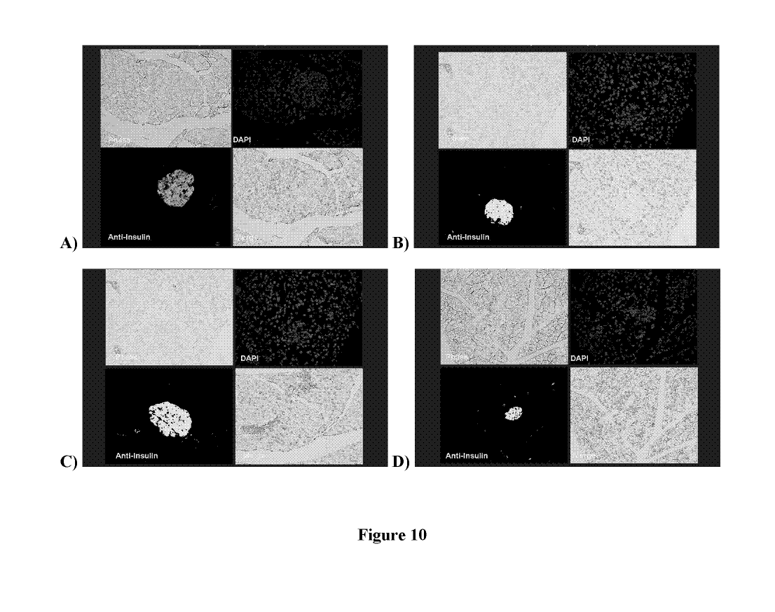

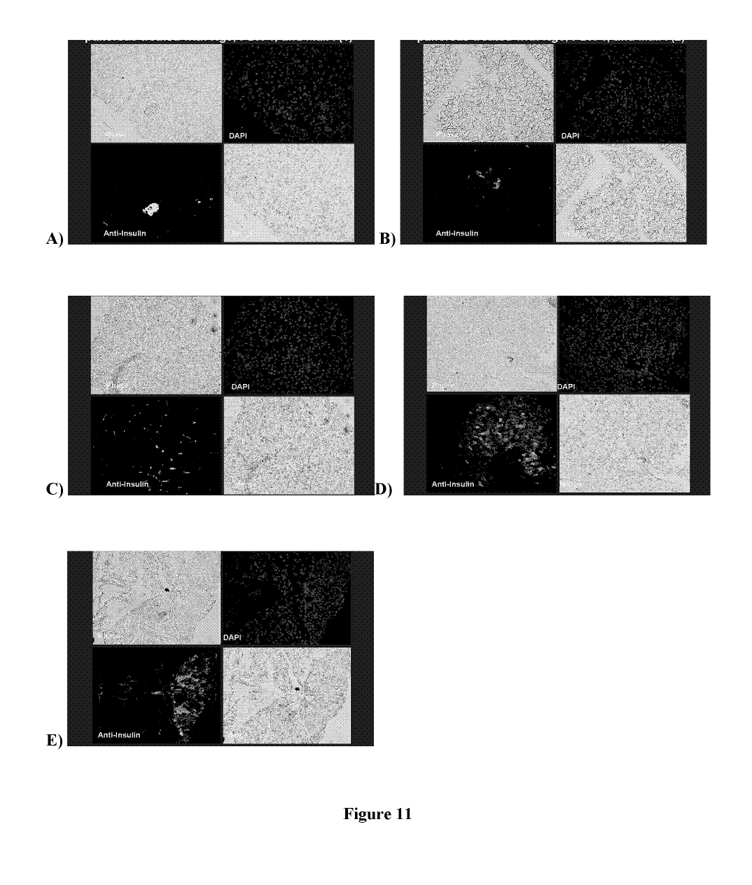

Six CD-1 mice (Charles River Laboratory) were divided into two groups: the treatment group and the control group. Transducible material His6-Ngn3-11R (1 mg/kg), His 6-PDX1-11R (1 mg/kg), and His6-MafA-11R (1 mg/kg) were injected into each mouse by intraperitoneal (IP) in treatment group (Mouse-4, Mouse-5 and Mouse-6) and BSA (1 mg/kg) was injected into each mouse in the control group (Mouse-1, Mouse-2 and Mouse-3). There was no Greenish-brown or Yellow aspirate when needle penetrated into each mouse peritonea. Injections were repeated every day for 7 days. Mice of both treatment and control group were sacrificed on the 3rd day after the completion of all injections. The mouse liver and pancreas were washed with 1×PBS and fixed by 4% paraformaldehyde for overnight. Then the liver and pancreatic tissues were processed by standard Paraffin Embedding protocol. The Tissue sections, 5-micro in thickness, were prepared routinely with histology microtomes and mounted on standard histology glass slides. The wax in tissues was dissolved by xylene during processing of tissue sections. Tissue sectioning and histologic and immunohistochemical staining were performed using routine methods. For indirect fluorescent-antibody (IFA) assay, the slides were blocked with 0.05% Tween-20 (TBST) and 3% BSA for 1 hour at RT and were incubated with mouse anti-insulin antibody (Invitrogen) at 4° C. overnight. The slides were washed three times with PBS for 15 minutes at RT and incubated with fluorescein isothiocyanate (FITC) conjugated swine anti-mouse antibody (KPL) for 2 hours at RT. Same concentration of Mouse IgG was used as isotype control. Anti-DAPI antibody was added to slides as a nuclear marker. The slides were washed as before and mounted with aqueous mounting media (Biomeda, Foster City, Calif.). Endothelial markers were identified under the microscope (Olympus BX51, San Diego, Calif.) and merged cells were analyzed by Microsuite Biological Suite program (Olympus BX51, San Diego, Calif.) (FIGS. 8-11). The results showed that the treatment group had more insulin-producing cells (FIG. 9) in livers comparing to the control group (FIG. 8). The pancreas of the control group showed a cluster of insulin-producing cells (FIG. 10), while the pancreas of the treatment group showed insulin-producing cells in bigger area (FIG. 11). Therefore, the results showed that treatment of transducible materials His6-Ngn3-11R, His 6-PDX1-11R, and His6-MafA-11R converted liver and/or pancreas cells to insulin-producing cells.

### Example 3

Reprogramming of T cells and programs them to Treg cells using transducible material Foxp3.

A poly-arginine protein transduction domain was fused to the C-terminal of each reprogramming protein Foxp3 through a linker (SEQ ID NO: 55) to form His6-Foxp3-11R (FIG. 7). His6 (SEQ ID NO: 59) was included to facilitate protein purification. The poly-arginine fused protein was expressed in E. Coli in inclusion body form, which were then solubilized, refolded, and further purified to prepare transducible materials His6-Foxp3-11R. The protein identities were confirmed by Western blot analysis.

100 ml of healthy human blood was collected from a donor and the peripheral blood mononuclear cells (PBMCs) were isolated by density-gradient centrifugation using Histopaque-1077 (Sigma-Aldrich, St Louis, Mo.). CD14+ monocytes were removed by magnetic bead selection (Miltenyi Biotec, Auburn, Calif.). Briefly, 108 PBMCs were incubated with 200 μL anti-CD14 microbeads (Miltenyi Biotec) in ice for 30 minutes. The cells were washed with cold 1×PBS with 2% FCS and centrifuged at 300 g for 10 minutes and then resuspended in 1×PBS with 2% FCS. The cell suspension was applied to the magnetic column and unbinding cells were passed through by washing 3 times with 1×PBS with 2% FCS. The PBMC/mono− were harvested by centrifuged at 300 g for 10 minutes.

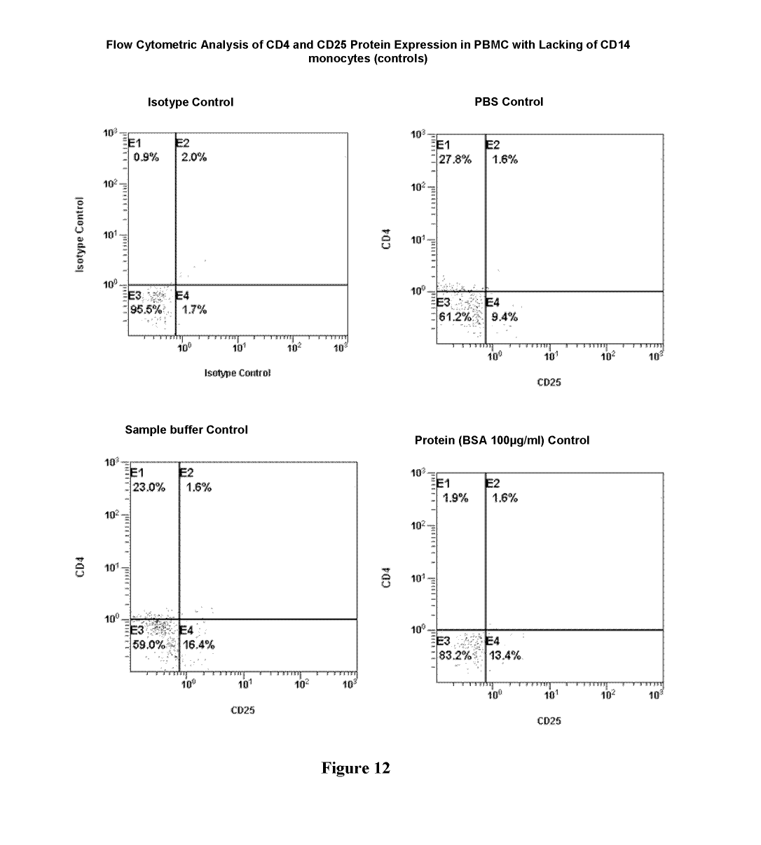

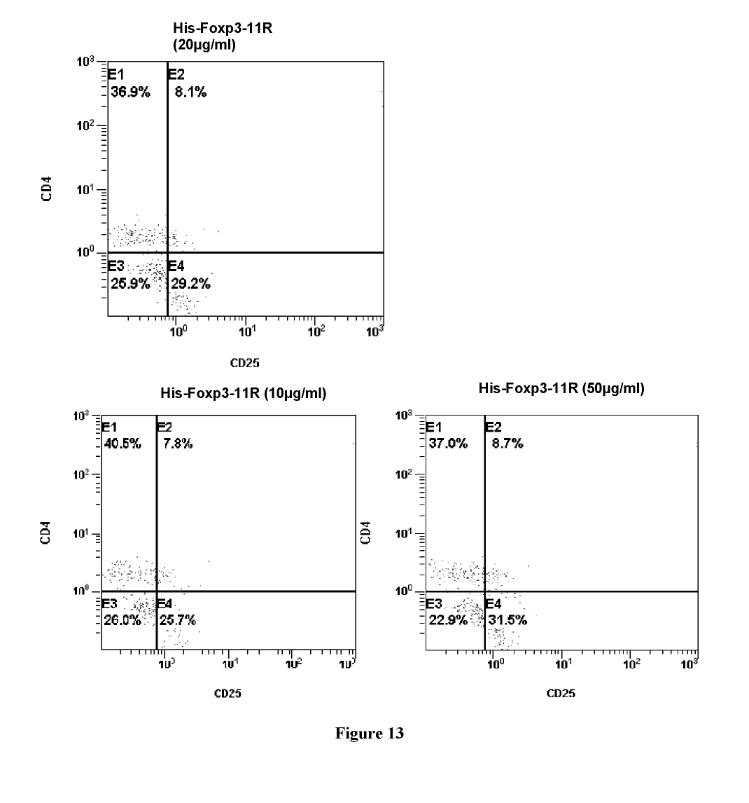

The PBMC/mono− were cultured in 6-well plates (Becton Dickinson, Gaithersburg, Md.) supplemented with 10% FBS, nonessential amino acids, 2 mM glutamine, 1 mM sodium pyruvate, 25 mM HEPES, 200 units/ml penicillin, and streptomycin at 37° C. and 5% CO₂. After 1 hour of culture, His6-Foxp3-11R (10 μg/ml, 20 μg/ml, or 50 μg/ml) was added to the cells. BSA (100 μg/ml) was added to another well as control. Same concentration of His6-Foxp3-11R or BSA was added after cultured for two days. After 5 days of culture, the cells were washed with PBS twice. The cells were re-suspended in 100 μL diluted and added rabbit anti-human CD25 for 90 minutes. The cells were washed three time with cold 1×PBS supplied 2% FBS and then the conjugated-PE mouse anti-human CD4 as well as conjugated-FITC goat anti-rabbit IgG were added to the cells for 60 minutes in ice. Conjugated-PE mouse IgG and rabbit IgG were incubated with another group cells as Isotype control. The cells were washed with PBS for flow cytometric analysis using a Beckman Coulter FC500 cytometer with Cytomics CXP software (Beckman Coulter, Fullerton, Calif.) (FIGS. 12 and 13). The results showed that the CD4+CD25+T cells (Treg cells) have dramatically increased with treatment of transducible material His6-Foxp3-11R, and the increase is protein-dose dependent.

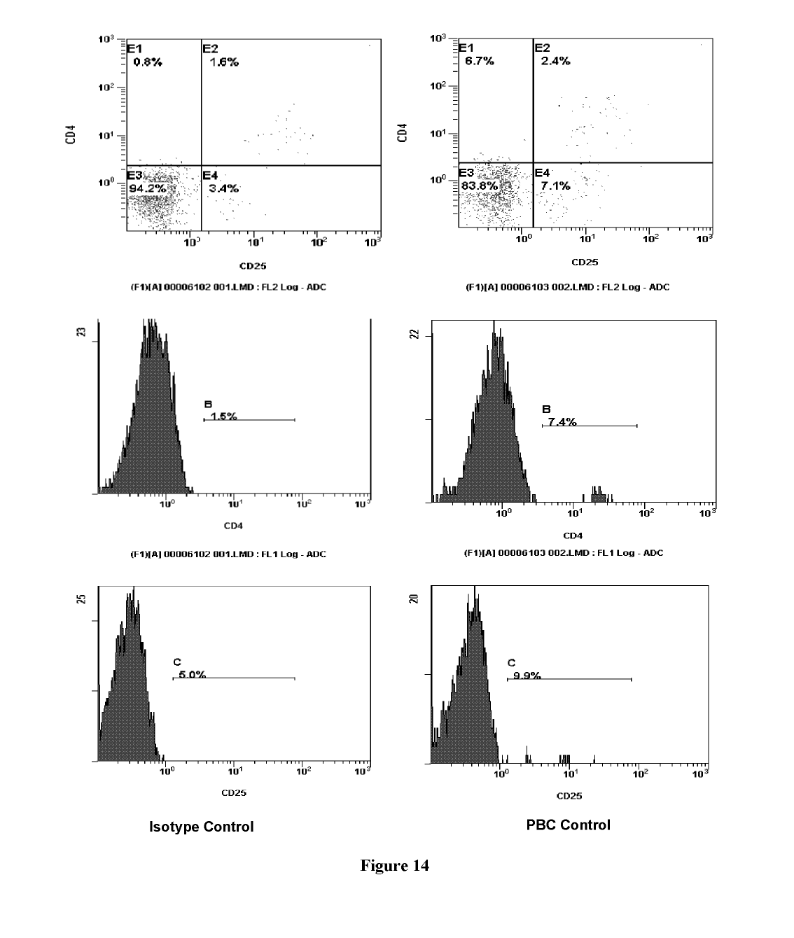

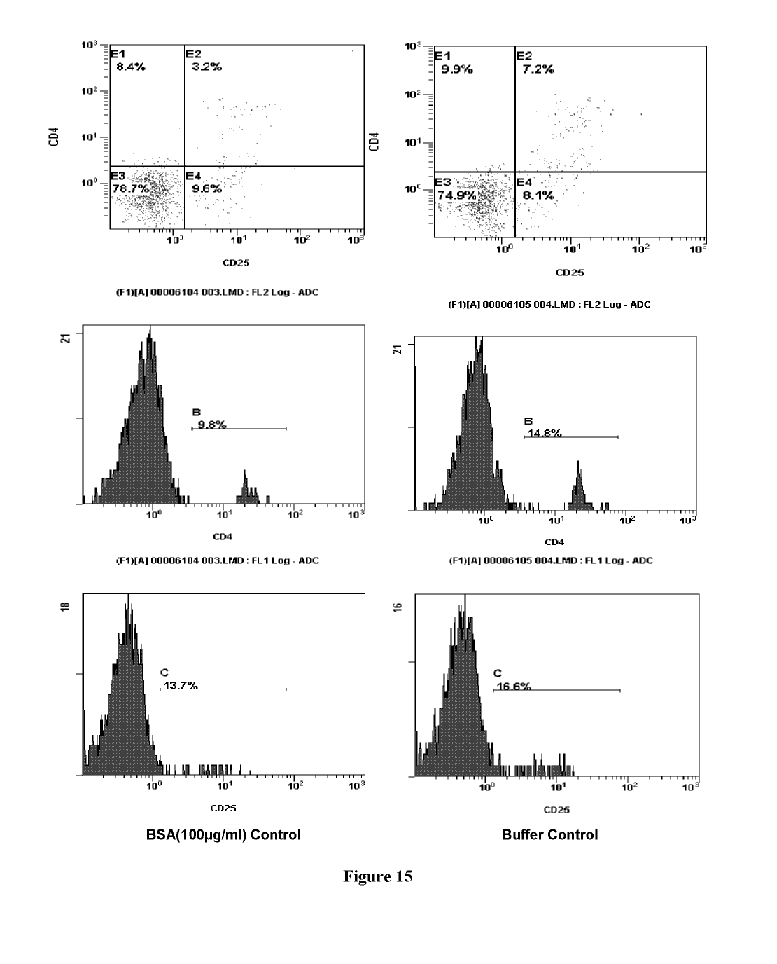

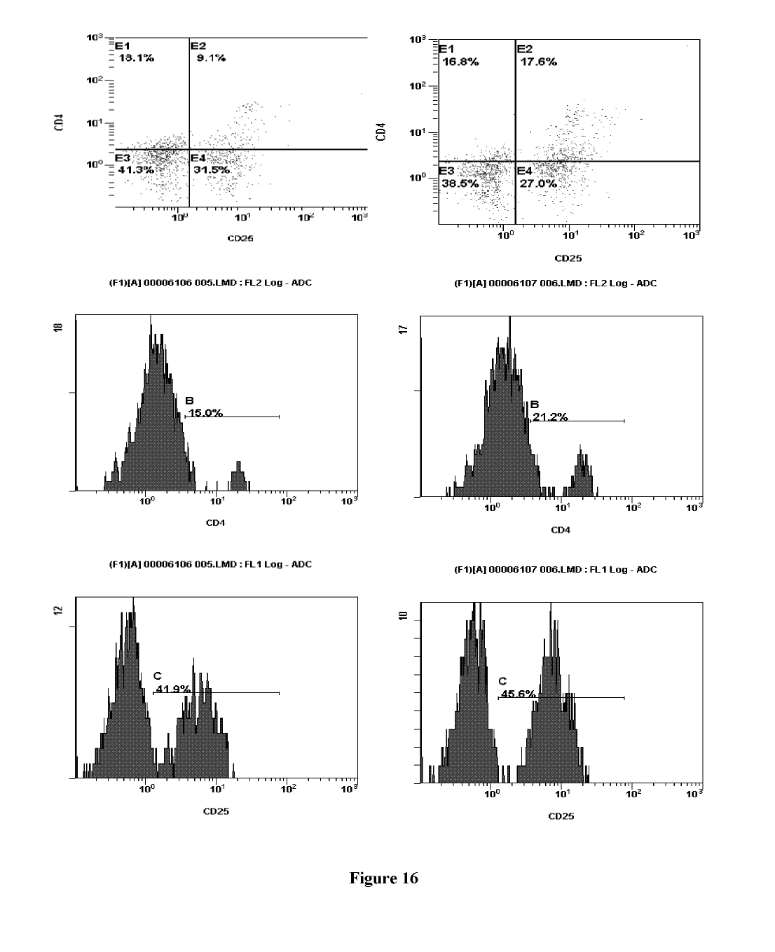

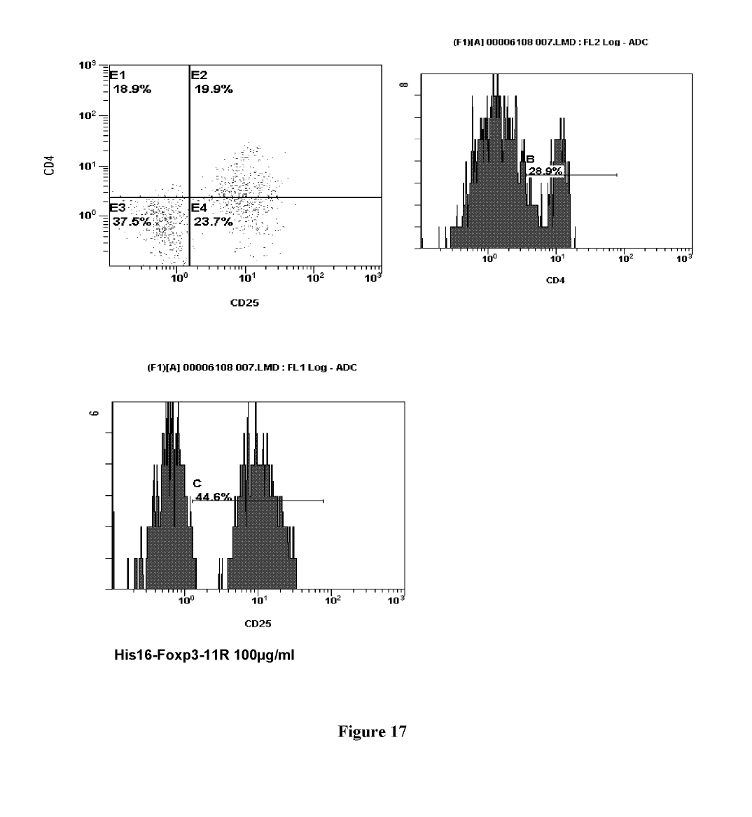

100 ml of healthy human blood was collected from a donor and the peripheral blood mononuclear cells (PBMCs) were isolated by density-gradient centrifugation using Histopaque-1077 (Sigma-Aldrich, St Louis, Mo.). The PBMC/mono⁻ were cultured in 6-well plates (Becton Dickinson, Gaithersburg, Md.) supplemented with 10% FBS, nonessential amino acids, 2 mM glutamine, 1 mM sodium pyruvate, 25 mM HEPES, 200 units/ml penicillin, and streptomycin at 37° C. and 5% CO2. After 1 hour of culture, Foxp3 (10 μg/ml, 50 μg/ml, 100 μg/ml) were added to the cells. BSA (100 μg/ml) was added to another well as control. Same concentration of the Foxp3 or BSA was added after cultured two days. Following 5 days of culture, the cells were washed with PBS twice. The cells were re-suspended in 100 μL diluted and added rabbit anti-human CD25 for 90 minutes. The cells were washed three time with cold 1×PBS supplied 2% FBS and then the conjugated-PE mouse anti-human CD4 as well as conjugated-FITC goat anti-rabbit IgG were added to the cells for 60 minutes in ice. Conjugated-PE mouse IgG and rabbit IgG were incubated with another group cells as Isotype control. The cells were washed with PBS for flow cytometric analysis using a Beckman Coulter FC500 cytometer with Cytomics CXP software (Beckman Coulter, Fullerton, Calif.) (FIGS. 14-17). The results showed that the CD4+CD25+T cells (Treg cells) have dramatically increased with treatment of transducible material His6-Foxp3-11R, and the increase is protein-dose dependent.

### Example 4

Reprogramming of bone marrow derived MSC and reprograms them to chondrogenic cells by supercharged Sox9.

A bioactive supercharged Sox9 (scSox9, also referred to as scGFP-Sox9-11R, the sequence of which is shown in FIG. 22(n)) was generated by fusing Sox9 with a super positively charged green fluorescence protein (scGFP) using molecular engineering technology. Briefly, the cDNA sequence encoding scSox9 was first optimized for high level protein expression in E. coli and then synthesized. Next, the gene was subcloned into vector pET-15b and this protein expression plasmid was then transformed into BL21 (DE3) competent cells. Finally the fusion protein was produced, refolded, and purified from E. coli culture.

Human skin fibroblast cell line (HHF) and human bone marrow derived mesenchymal stem cells (MSC) at passage 5 were cultured with 10 μg/ml scSox9 or with 10 μg/ml scGFP (negative control) in monolayer or in cell aggregate in DMEM medium at 37° C. After one hour incubation with scSox9, cells were washed and viewed under fluorescent microscope. Both HHF cells and MSC showed intracellular expression of green fluorescence, indicating entry of scSox9 into these cells (FIG. 23).

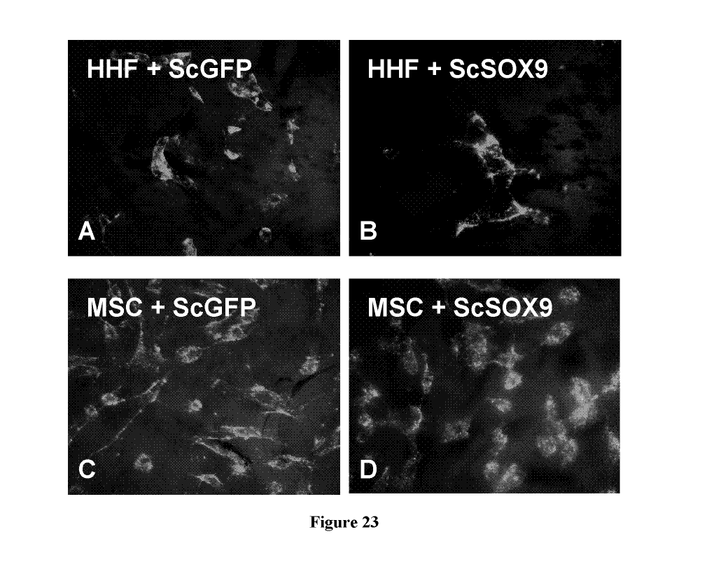

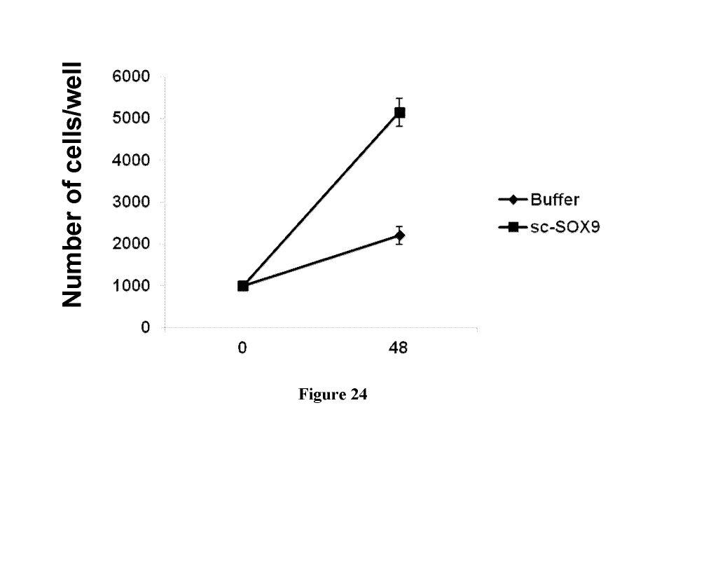

MSC were cultured in DMEM containing 10% FBS and low glucose (1.5 g/l) with addition of scSox9 or buffer. The number of scSox9 treated MSC was increased two folds after 72 hours in culture compared with buffer treated MSC (FIG. 24). The results showed that scSox9 was able to induce MSC proliferation and differentiation with no requirement of additional growth factors.

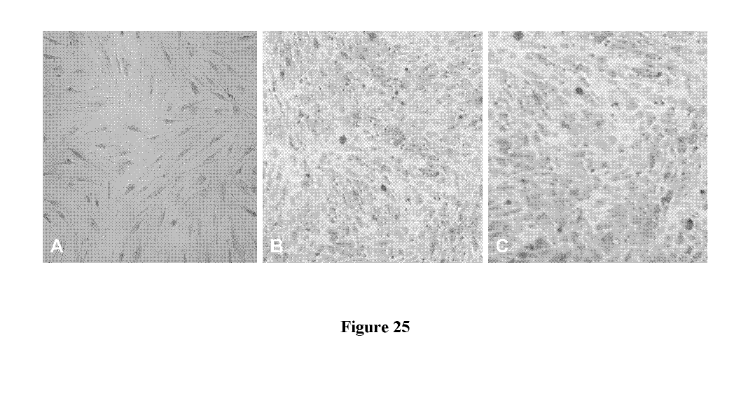

Next, the differentiation of MSC into chondrocytes was investigated. MSC were cultured in DMEM containing 1% FBS and high glucose (4.5 g/l) and 10 μg/ml of scSox9 was added. After 24 hours, culture medium was changed to DMEM containing 0% FBS and high glucose with no additional scSox9 was added. Culture was maintained for 14 days. As early as 48 hours, scSox9 treated MSC started to change morphology into chondrocyte-like cells and this morphology maintained for 21 days in culture. These morphologically changed cells stained positive for toluidine blue, which demonstrated production of aggrecan, which is a major component of proteoglycan in articular cartilage matrix, and chondrocytes' functions (FIG. 25). The results showed that scSox9 induced MSC to differentiate into chondrocytes.

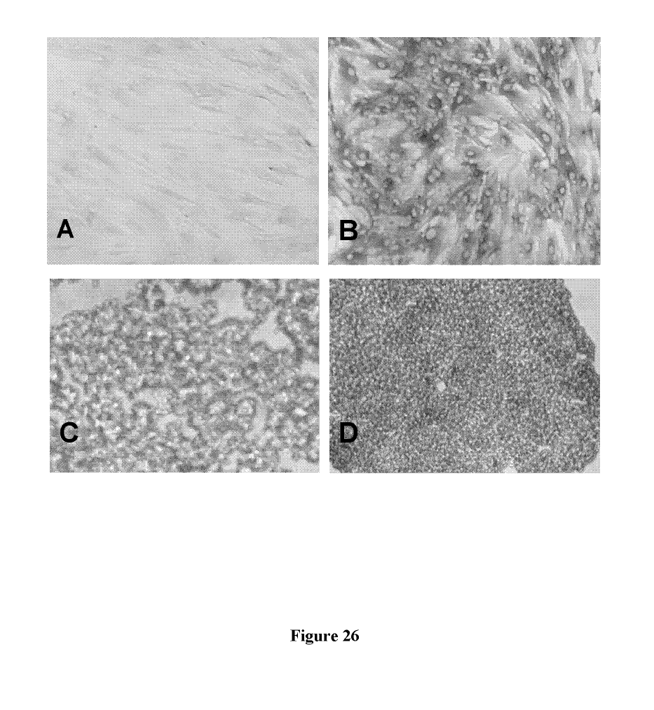

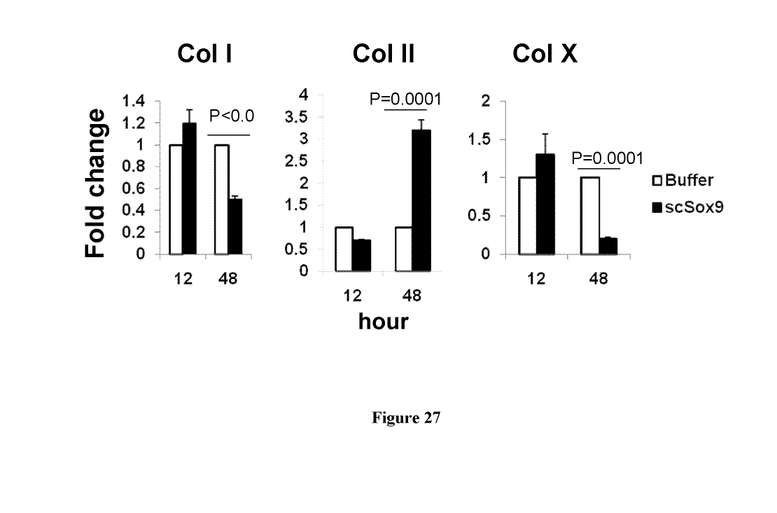

Furthermore, scSox9 induced collagen type II expression and down-regulated collagen type I and type X production (FIGS. 26-27). Human bone marrow derived MSC was cultured with 10 μg/ml of scGFP (FIGS. 26 A and C) or scSox9 (FIGS. 26 B and D) in monolayer (FIGS. 26 A and B) or aggregate (FIGS. 26 C and D) for chondrogenesis. At day 14, aggregates were harvested and snap-frozen. Cryostat sections were stained with a mouse anti-human collage type II monoclonal antibody (Immunoperoxidase staining). As shown in FIG. 26, MSC cultured with scGFP only showed poorly formed aggregate, but MSC cultured with scSox9 were positive for collagen II, and showed good formation of aggregate.

MSC were cultured with DMEM containing 1% FBS and high glucose (4.5 g/l) with addition of buffer only or 10 μg/ml of scSox9. At 12 hours and 48 hours after the treatment, RNA was extracted and RT-PCR was performed with TaqMan probe based analysis assay for collagen (Col) type I, II and X mRNA expression. Expression as relative to GAPDH. As shown in FIG. 27, scSox9 induced increased collagen type II but decreased collagen type I and type X expression.

Type X collagen is typically expressed in hypertrophic chondrocytes, and Type II collagen in fibrocartilage, whereas the superior hyaline cartilage expresses Type I collagen. The data indicated that while promoting chondrogenesis, scSox9 can concomitantly repress chondrocyte hypertrophy and inhibit osteogenesis. Most importantly, one time addition of scSox9 was sufficient to induce MSC differentiation into chondrocytes and maintained the chondrocyte phenotype.

These in vitro data demonstrated that scSox9 is able to enter bone marrow derived MSC and induce chondrogenesis and maintain chondrocyte phenotype.

1. A transducible material comprising an effector domain, wherein the effector domain is a polypeptide, a small molecule, or a polynucleotide, and wherein the polypeptide is selected from the group consisting of Sox9, Sox6, Sox5, c-Myc, Klf4, Mef2C, Trps1, Gli3, Runx2, Dlx5, Dlx6, GATA-6, and Baf60c, and a homologous sequence thereof, and any combination thereof.

2. The transducible material of claim 1, further comprising a protein selected from the group consisting of Oct4, Klf4, Lin28, Nanog, cMyc, Ngn3, PDX1, MafA, NeuroD, Foxp3, Foxo1, Foxo3, a homologous sequence thereof, and any combination thereof.

3. The transducible material of claim 1, wherein the homologous sequence means a sequence sharing at least 70% of identity in amino acid sequence with at least one member in the group.

4. The transducible material of claim 3, wherein the homologous sequence has substantially the same activity as at least one member in the group.

5. The transducible material of claim 1 further comprising a transduction domain.

6. The transducible material of claim 5, wherein the transduction domain is linked to the effector domain covalently, non-covalently or via a linker.

7. The transducible material of claim 1, wherein the effector domain is inherently transducible.

8. The transducible material of claim 1, wherein the effector domain is selected from the group consisting of Sox9, Sox6, Sox5, c-Myc, Klf4, Mef2C, Trps1, Gli3, Runx2, Dlx5, Dlx6, GATA-6, Baf60c, a homologous sequence thereof, and any combination thereof.

9. The transducible material of claim 5, wherein the transduction domain is selected from the group consisting of a protein transduction domain, a cell penetrating peptide, a cell permeating peptide, an activatable cell penetrating peptide, a cell-targeting peptide, a polymer, and a supercharged protein.

10. The transducible material of claim 9, wherein the protein transduction domain is selected from the group consisting of TAT, poly-arginine, Penetratin, Antennapedia, VP22, Transportan, MAP, MTS, PEP-1, Arg/Trp analogue, RRWRRWWRRWWRRW, polyguanidine peptoid, polyguanidine peptoid, inherent protein transduction domain, SEQ ID NO: 56, SEQ ID NO: 57, HIV-1 Rev, Flock house virus coat peptide, DNA-binding peptides, c-Fos, c-Jun, yeast GCN4, Fusogenic HA2 peptide and supercharged GFP.

11. The transducible material of claim 9, wherein the cell-targeting peptide is a peptide having an amino acid sequence selected from the group consisting of NGR, RGD, SEQ ID NO: 45, SEQ ID NO: 46, SEQ ID NO: 47, SEQ ID NO: 48, SEQ ID NO: 49, SEQ ID NO: 50, SEQ ID NO: 51, SEQ ID NO: 52, SEQ ID NO: 53, SEQ ID NO: 54, and SEQ ID NO: 58.

12. The transducible material of claim 9, wherein the polymer is selected from the group consisting of a cationic lipid polymer and a nanoparticle.

13. The transducible material of claim 1, wherein the transducible material is capable of selectively transducing into one or more specific biological samples or capable of becoming transducible in a specific environment surrounding the biological sample.

14. The transducible material of claim 6, wherein the linker has an amino acid sequence set forth in SEQ ID: 55.

15. The transducible material of claim 14, wherein the protein transduction domain is poly-arginine.

16. The transducible material of claim 5, further comprising one or more motifs that do not interrupt the function of the effector domain or the transduction domain.

17. The transducible material of claim 16 wherein the motif is covalently linked to the effector domain or the transduction domain.

18. The transducible material of claim 17, wherein the motif has an amino acid sequence set forth in SEQ ID: 59.

19. A method of reprogramming a biological sample, comprising:
exposing the biological sample to at least one transducible material of claim 1.

20. The method of claim 19 wherein the biological sample is a cell, a tissue, or an organ from a biological organism.

21. The method of claim 20 wherein the biological organism is a microorganism, a plant or an animal.

22. The method of claim 19 wherein the biological sample is reprogrammed so as to cause proliferation, differentiation, transdifferentiation, retrodifferentiation, transdertermination, dedifferentiation, apoptosis or morphogenesis.

23. The method of claim 20 wherein the cell is reprogrammed to change from a starting cell to a chondrogenic cell.

24. The method of claim 23 wherein the starting cell is selected from the group consisting of an embryonic stem cell (ESC), an induced pluripotent stem cell (iPSC), an MSC, a fibroblast, a hematopoietic stem cell, an endothelian stem cell, an adipocyte, a chondrocyte, an osteoblast, an osteoclast and endothelial cell.

25. The method of claim 24, wherein the starting cell is an embryonic stem cell (ESC), an induced pluripotent stem cell (iPSCs), or an MSC.

26. The method of claim 24, wherein the transducible material comprises a polypeptide selected from the group consisting of: Sox9, Sox6, Sox5, c-Myc, Klf4, Mef2C, Trps1, Gli3, Runx2, Dlx5, Dlx6, GATA-6, Baf60c, Sox9-11R, Sox6-11R, Sox5-11R, c-Myc-11R, Klf4-11R, Mef2C-11R, Trps1-11R, Gli3-11R, Runx2-11R, Dlx5-11R, Dlx6-11R, GATA-6-11R, Baf60c-11R, His6-Sox9-11R, His6-Sox6-11R, His6-Sox5-11R, His6-c-Myc-11R, His6-Klf4-11R, His6-Mef2C-11R, His 6-Trps1-11R, His6-Gli3-11R, His6-Runx2-11R, His6-Dlx5-11R, His6-Dlx6-11R, His6-GATA-6-11R, His6-Baf60c-11R, HA2-Supercharged GFP-Sox9, HA2-Supercharged GFP-Sox6, HA2-Supercharged GFP-Sox5, HA2-Supercharged GFP-c-Myc, HA2-Supercharged GFP-Klf4, HA2-Supercharged GFP-Mef2C, HA2-Supercharged GFP-Trps1, HA2-Supercharged GFP-Gli3, HA2-Supercharged GFP-Runx2, HA2-Supercharged GFP-Dlx5, HA2-Supercharged GFP-Dlx6, HA2-Supercharged GFP-GATA-6, HA2-Supercharged GFP-Baf60c, scGFP-Sox9-11R, scGFP-Sox6-11R, scGFP-Sox5-11R, scGFP-c-Myc-11R, scGFP-Klf4-11R, scGFP-Mef2C-11R, scGFP-Trps1-11R, scGFP-Gli3-11R, scGFP-Runx2-11R, scGFP-Dlx5-11R, scGFP-Dlx6-11R, scGFP-GATA-6-11R, and scGFP-Baf60c-11R.

27. A pharmaceutical composition comprising a transducible material of claim 1.

28. The pharmaceutical composition of claim 27 further comprising an epigenetic agent.

29. The pharmaceutical composition of claim 28, wherein the epigenetic agent comprises trichostatin A, valproic acid, aza-2′-deoxycytidine, or suberoylanilide hydroxamic acid.

30. A composition comprising a biological sample and a transducible material of claim 1, wherein the transducible material has transduced into the biological sample.

31. Use of a transducible material of claim 1 in manufacturing a medicament for treating a condition involving cartilage damage in a biological organism.

32. The use of claim 31 wherein the condition involving cartilage damage is joint disorder, osteoarthritis, cartilage injury, traumatic rupture or detachment, achondroplasia, costochondritis, spinal disc herniation, relapsing polychonritis, tumor, chondroma, chondrosarcoma, or peomorphic adenoma.

33. A method of treating a disease or condition involving cartilage damage of a biological organism comprising:
removing a biological sample from the biological organism;
exposing the biological sample to a transducible material of claim 1; and
transplanting the biological sample transduced with the transducible material back to the biological organism.

34. A method of developing cell-based therapies for diseases or conditions involving cartilage damage comprising:
reprogramming an iPSC, an embryonic stem cell, a progenitor cell or a differentiated cell to a transplantable chondrogenic cell by exposing the iPSC, the embryonic stem cell, the progenitor cell or the differentiated cell to at least one transducible material of claim 1;
transplanting the transplantable chondrogenic cell into a biological sample or a biological organism; and
assessing the therapeutic effects of the transplantable chondrogenic cell.

35. A method of developing a disease model comprising:
exposing an iPSC, an embryonic stem cell, a progenitor cell or a differentiated cell to at least one transducible material of claim 1 so as to reprogram into a transplantable chondrogenic cell;
transplanting the transplantable chondrogenic cell into a biological sample or a biological organism;
developing a disease model having the transplantable chondrogenic cell.

36. A method of developing drug screening or toxicity models comprising:
reprogramming an iPSC, an embryonic stem cell, a progenitor cell or a differentiated cell to a chondrogenic cell via exposing to at least one transducible material of claim 1; and
using the chondrogenic cell to screen the effects and/or toxicities of different compounds.

37. A method of identify an effector domain comprising:
covalently linking a test effector domain to a transduction domain to form a test transducible molecule;
exposing the test molecule to a biological sample, and
measuring a reprogramming level of the biological sample to a chondrogenic sample.
